(19) United States
(12) Patent Application Publication
(10) Pub. No.: US 2022/0241263 A1
(43) Pub. Date: Aug. 4, 2022

(54) PD-1 AXIS BINDING ANTAGONIST TO TREAT CANCER WITH GENETIC MUTATIONS IN SPECIFIC GENES

(71) Applicants: Ares Trading S.A., Aubonne (CH); Pfizer Inc., New York, NY (US)

(72) Inventors: Marat Alimzhanov, Wellesley, MA (US); Keith Anthony Ching, San Diego, CA (US); Alessandra Di Pietro, Milano (IT); Xinmeng Mu, San Diego, CA (US); Paul Brian Robbins, San Diego, CA (US)

(21) Appl. No.: 17/615,735

(22) PCT Filed: May 29, 2020
(86) PCT No.: PCT/EP2020/065038

§ 371 Date: Dec. 1, 2021

Related U.S. Application Data
(60) Provisional application No. 62/855,923, filed on Jun. 1, 2019.
(60) Provisional application No. 62/927,963, filed on Oct. 30, 2019.
(60) Provisional application No. 63/013,132, filed on Apr. 21, 2020.

(51) Int. Cl. A61K 31/4439; A61K 39/395; C07K 16/28; A61P 35/00

(52) U.S. Cl. CPC A61K 31/4439; A61P 35/00; C07K 16/2818; A61K 39/3955

(57) ABSTRACT

The present disclosure describes combination therapies comprising a PD-1 axis binding antagonist, wherein the cancer has been pre-determined to have one or more genetic mutations in one or more genes, to have certain gene expression profiles, and/or to have other biomarkers.

### FIELD

The present invention relates to a PD-1 axis binding antagonist to treat cancer in patients with certain pre-determined genetic mutations, gene expression profiles, and/or other biomarkers and methods and uses thereof.

### BACKGROUND

The programmed death 1 (PD-1) receptor and PD-1 ligands 1 and 2 (PD-L1 and PD-L2, respectively), play integral roles in immune regulation. Expressed on activated T cells, PD-1 is activated by PD-L1 (also known as B7-H1) and PD-L2 expressed by stromal cells, tumor cells, or both, initiating T-cell death and localized immune suppression (Dong et al., Nat Med 1999; 5:1365-69; Freeman et al. J Exp Med 2000; 192:1027-34), potentially providing an immune-tolerant environment for tumor development and growth. Conversely, inhibition of this interaction, by a PD-1 axis binding antagonist, Several PD-axis binding antagonists, including the PD-1 antibodies nivolumab (Opdivo), pembrolizumab (Keytruda) and PD-L1 antibodies avelumab (Bavencio), durvalumab (Imfinzi), and atezolizumab (Tecentriq) were approved by the U.S. Food and Drug Administration (FDA) for the treatment of cancer in recent years can enhance local T-cell responses and mediate antitumor activity in nonclinical animal models (Iwai Y, et al. Proc Natl Acad Sci USA 2002; 99:12293-97). A recent phase 3 clinical trial, (clinicaltrials.gov number NCT02684006) showed patients with advanced renal cell carcinoma (RCC) who received avelumab in combination with axitinib (Inlyta®), a VEGFR inhibitor and an FDA approved drug for the treatment of advanced RCC after failure of one prior systemic therapy, had a better median progression-free survival (13.8 months) and objective response rate (55.2%) than patients who received sunitinib, a standard of care treatment of advanced RCC. N Engl J Med 2019; 380:1103-15. Avelumab was subsequently approved by the FDA for use in combination with axitinib to treat advanced RCC in the first line setting. Despite of all the new cancer therapies in the recent years, there remains a need of improved therapies for the treatment of cancers. Furthermore, there is a need for therapies having greater efficacy than existing therapies, and particularly in selected patient populations.

### SUMMARY

In one embodiment, the invention provides a method for treating cancer in a patient, wherein the cancer in the patient is pre-determined to contain one or more protein altering mutations in one or more genes, the method comprising administering to the patient a therapeutically effective amount of a PD-1 axis binding antagonist. In some embodiments, the genes may be selected from the group consisting of CD163L1, DNMT1, MCR1R, FOXO1, STAB2, LOC728763, MYH7B, IL16, SPATA31C2 and ABCA1. In some embodiments, the cancer of the patient is pre-determined not to contain a protein altering mutation in the PTEN gene. In some embodiments, the cancer of the patient is pre-determined not to contain a protein altering mutation in the PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, or PTPDC1 gene. In one aspect, and in combination of any other aspect not inconsistent, the method further comprises administering to the patient a therapeutically effective amount of a VEGF pathway inhibitor.

In another embodiment, the invention provides a medicament comprising a PD-1 axis binding antagonist for use in treating a cancer in a patient, wherein the cancer of the patient is pre-determined as containing one or more protein altering mutations in one or more genes. In some embodiments, the gene(s) may be selected from the group consisting of CD163L1, DNMT1, MCR1R, FOXO1, STAB2, LOC728763, MYH7B, IL16, SPATA31C2 and ABCA1. In some embodiments, the cancer of the patient is pre-determined not to contain a protein altering mutation in the PTEN gene. In some embodiments, the cancer of the patient is pre-determined not to contain a protein altering mutation in the PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, or PTPDC1 gene. In one aspect, and in combination of any other aspect not inconsistent, the medicament is to be used in combination with a VEGF pathway inhibitor.

In another embodiment, the invention provides a kit which comprises a first container, a second container and a package insert, wherein the first container comprises at least one dose of a medicament comprising an PD-1 axis binding antagonist, the second container comprises at least one dose of a medicament comprising a VEGF pathway inhibitor, and the package insert comprises instructions for treating a subject for cancer wherein the cancer is pre-determined to contain one or more protein altering genetic mutations in one or more genes, using the medicaments. In some embodiments, the gene(s) may be selected from the group consisting of CD163L1, DNMT1, MC1R, FOXO1, STAB2, LOC728763, MYH7B, IL16, SPATA31C2 and ABCA1. In some embodiments, the package insert comprises instructions for treating a patient for cancer wherein the cancer of the patient is pre-determined not to contain a protein altering mutation in the PTEN gene. In some embodiments, the cancer of the patient is pre-determined not to contain a protein altering mutation in the PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, or PTPDC1 gene.

In another embodiment, the invention provides a method for improving progression free survival (PFS) of a patient suffering from cancer comprising administering to the patient an effective amount of a PD-1 axis binding antagonist, wherein the cancer of the patient contains one or more protein altering mutations in one or more genes. In some embodiments, the gene(s) may be selected from the group consisting of CD163L1, DNMT1, MC1R, FOXO1, STAB2, LOC728763, MYH7B, IL16, SPATA31C2 and ABCA1. In some embodiments, the cancer of the patient is pre-determined not to contain a protein altering mutation in the PTEN gene. In some embodiments, the cancer of the patient is pre-determined not to contain a protein altering mutation in the PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, or PTPDC1 gene. In some embodiments, the PFS of the patient is improved over patients suffering from cancer but which cancer does not contain any protein altering mutations in CD163L1, DNMT1, MC1R, FOXO1, STAB2, LOC728763, MYH7B, IL16, SPATA31C2 or ABCA1, or contains a protein altering mutation in the PTEN gene. In one aspect, and in combination of any other aspect not inconsistent, the method further comprises administering to the patient a therapeutically effective amount of a VEGF pathway inhibitor.

In another embodiment, provided herein is a method of treating a patient having a cancer, comprising administering to the patient a therapeutically effective amount of a PD-1 axis binding antagonist, wherein the expression level of the gene UTS2 in a sample obtained from the patient has been determined to be increased as compared to a reference level. Optionally, the method further comprises administering to the patient a therapeutically effective amount of a VEGF pathway inhibitor.

In another embodiment, provided herein is a method of treating a patient having a cancer, comprising administering to the patient a therapeutically effective amount of a PD-1 axis binding antagonist, wherein the expression level of at least 1, 2, 3, 4, 5, 6, 7, 8, 10, 15, 20, 25 or all 26 genes selected from the group consisting of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1 in a sample obtained from the patient has been determined to be increased as compared to a reference level. Optionally, the method further comprises administering to the patient a therapeutically effective amount of a VEGF pathway inhibitor.

In another embodiment, provided herein is a method of identifying a patient having a cancer who may benefit from a treatment comprising a therapeutically effective amount of a PD-1 axis binding antagonist, the method comprising determining an expression level of the gene DUX4 or the DUX4 gene signature in a sample obtained from the patient, wherein an increased expression level of the gene DUX4 or the DUX4 gene signature in the sample as compared to a reference level identifies the patient as one who has a decreased likelihood of benefiting from a treatment comprising a therapeutically effective amount of a PD-1 axis binding antagonist.

In another embodiment, provided herein is a method of identifying a patient having a cancer who may benefit from a treatment comprising a therapeutically effective amount of a PD-1 axis binding antagonist, the method comprising determining an expression level of at least 1, 2, 3, 4, 5, 6, 7, 8, 10, 15, 20, 25 or 26 genes selected from the group consisting of NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB in a sample obtained from the patient, wherein an increased expression level of the at least 1, 2, 3, 4, 5, 6, 7, 8, 10, 15, 20, 25 or 26 genes in the sample as compared to a reference level identifies the patient as one who has a decreased likelihood of benefiting from a treatment comprising a therapeutically effective amount of a PD-1 axis binding antagonist.

In another embodiment, provided herein is a method for treating cancer in a patient, wherein the cancer in the patient is pre-determined to have at least one of and optionally two, three, four, five, six, or all seven of the following characteristics:

(i) it contains one or more protein altering mutations in one or more gene(s) selected from the group consisting of CD163L1, DNMT1, MCI R, FOXO1, STAB2, LOC728763, MYH7B, IL16, SPATA31C2 and ABCA1;

(ii) it does not contain a protein altering mutation of the PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, or PTPDC1 gene;

(iii) it has an increased expression level of the gene UTS2 as compared to a reference level;

(iv) it has an increased expression level of at least one gene selected from the group consisting of at least 1, 2, 3, 4, 5, 6, 7, 8, 10, 15, 20, 25 of or all 26 genes selected from the group consisting of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCL5, XCL2, CST7, GFI1, KCNA3, PSTPIP1 as compared to a reference level;

(v) it does not have an increased expression level of the gene DUX4 or a DUX4 gene signature as compared to a reference level;

(vi) it does not have an increased expression level of at least one gene selected from the group consisting of at least 1, 2, 3, 4, 5, 6, 7, 8, 10, 15, 20, 25 of or all 26 genes selected from the group consisting of NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB as compared to a reference level; or

(vii) it contains one or more protein altering mutations in the NOTCH2 gene, the method comprising administering to the patient a therapeutically effective amount of a PD-1 axis binding antagonist. Optionally, the method further comprises administering to the patient a therapeutically effective amount of a VEGF pathway inhibitor.

In another embodiment, provided herein is a method for treating cancer in a patient, the method comprising:

(a) determining whether the cancer in the patient has at least one and optionally two, three, four, five, six, or all seven of the following characteristics: (i) it contains one or more protein altering mutations in one or more gene(s) selected from the group consisting of CD163L1, DNMT1, MC1R, FOXO1, STAB2, LOC728763, MYH7B, IL16, SPATA31C2 and ABCA1; (ii) it does not contain a protein altering mutation of the PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLK5, PTPN13, RTL1, SCAP, SHROOM2, SLCO2B1, TBX2, TENM3, TNRC6A, TTC28, USP42, ZC3H3, EFCAB6, MAP3K6, or PTPDC1 gene; (iii) it has an increased expression level of the gene UTS2 as compared to a reference level; (iv) it has an increased expression level of at least one gene selected from the group consisting of at least 1, 2, 3, 4, 5, 6, 7, 8, 10, 15, 20, 25 of or all 26 genes selected from the group consisting of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1 as compared to a reference level; (v) it does not have an increased expression level of the gene DUX4 or a DUX4 gene signature as compared to a reference level; (vi) it does not have an increased expression level of at least one gene selected from the group consisting of at least 1, 2, 3, 4, 5, 6, 7, 8, 10, 15, 20, 25 of or all 26 genes selected from the group consisting of NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB as compared to a reference level; or (vii) it contains one or more protein altering mutations in the NOTCH2 gene, and

(b) if the cancer in the patient has at least one or optionally two, three, four, five, six, or all seven of characteristics (i)-(vii), administering to the patient a therapeutically effective amount of a PD-1 axis binding antagonist. Optionally, the method further comprises administering to the patient a therapeutically effective amount of a VEGF pathway inhibitor.

In another embodiment, provided herein is a medicament comprising a PD-1 axis binding antagonist for use in treating a cancer in a patient, wherein a sample from the patient is pre-determined to have at least one of and optionally two, three, four, five, six, or all seven of the following characteristics:
(i) it contains one or more protein altering mutations in one or more gene(s) selected from the group consisting of CD163L1, DNMT1, MC1R, FOXO1, STAB2, LOC728763, MYH7B, IL16, SPATA31C2 and ABCA1;
(ii) it does not contain a protein altering mutation of the PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLK5, PTPN13, RTL1, SCAP, SHROOM2, SLCO2B1, TBX2, TENM3, TNRC6A, TTC28, USP42, ZC3H3, EFCAB6, MAP3K6, or PTPDC1 gene;
(iii) it has an increased expression level of the gene UTS2 as compared to a reference level;
(iv) it has an increased expression level of at least one gene selected from the group consisting of at least 1, 2, 3, 4, 5, 6, 7, 8, 10, 15, 20, 25 of or all 26 genes selected from the group consisting of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1 as compared to a reference level;
(v) it does not have an increased expression level of the gene DUX4 or a DUX4 gene signature as compared to a reference level;
(vi) it does not have an increased expression level of at least one gene selected from the group consisting of at least 1, 2, 3, 4, 5, 6, 7, 8, 10, 15, 20, 25 of or all 26 genes selected from the group consisting of NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB as compared to a reference level; or
(vii) it contains one or more protein altering mutations in the NOTCH2 gene.
   Optionally, the medicament further comprises a therapeutically effective amount of a VEGF pathway inhibitor.

In another embodiment, provided herein is a method of treating a patient having a cancer, comprising administering to the patient a therapeutically effective amount of a PD-1 axis binding antagonist, wherein the expression level of the gene UTS2 or of at least 1 , 2, 3, 4, 5, 6, 7, 8, 10, 15, 20, 25 or all 26 genes selected from the group consisting of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCL5, XCL2, CST7, GFI1, KCNA3, PSTPIP1 in a sample obtained from the patient has been determined to be increased as compared to a reference level, and wherein the expression level of DUX4 or a DUX4 gene signature or at least 1 , 2, 3, 4, 5, 6, 7, 8, 10, 15, 20, 25 or all 26 of the genes selected from the group consisting of NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB in a sample obtained from the patient has been determined to be decreased as compared to a reference level. Optionally, the method further comprises administering to the patient a therapeutically effective amount of a VEGF pathway inhibitor.

In one aspect of any of the embodiments described above, and in combination of any other aspects not inconsistent, the VEGF pathway inhibitor is a VEGFR inhibitor. In some cases, the VEGF pathway inhibitor is axitinib or a pharmaceutically acceptable salt thereof. In some cases, the VEGF pathway inhibitor is axitinib.

In another aspect of any of the embodiments described above, and in combination of any other aspect not inconsistent, the cancer in the patient does not contain, or is pre-determined not to contain a protein altering mutation of the PTEN gene. In some embodiments, the cancer does not contain any protein altering mutations of the PTEN gene listed in Table 1.11. In another aspect of any of the embodiments described above, and in combination of any other aspect not inconsistent, the cancer in the patient does not contain, or is pre-determined not to contain a protein altering mutation of the PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, or PTPDC1 gene.

In another aspect of the embodiments described above, and in combination of any other aspect not inconsistent, the pre-determination of a genetic mutation comprises genetic testing of a tumor tissue sample of the cancer patient.

In another aspect of the embodiments described above, and in combination of any other aspect not inconsistent, the cancer in the patient is pre-determined to contain one or more protein altering mutations in one or more genes. In some embodiments, the gene(s) may be selected from the group consisting of CD163L1, DNMT1 and MC1R. In some embodiments, the protein altering mutation of CD163L1, DNMT1, MC1R, ABCA1, FOXO1, IL16, MYH7B, STAB2, LOC728763, SPATA31C2, and PTEN is a mutation listed in Tables 1.1, 1.2, 1.3, 1.4, 1.5, 1.6(A)/1.6(B), 1.7, 1.8, 1.9, 1.10 and 1.11, respectively.

In another aspect of the embodiments described above, and in combination of any other aspect not inconsistent, the PD-1 axis binding antagonist is an anti-PD-1 antibody. In one embodiment, the anti-PD-1 antibody is selected from the group consisting of pembrolizumab, nivolumab, cemiplimab and RN888. In another embodiment, the anti-PD-1 antibody comprises
(a) a full length heavy chain having an amino acid sequence of SEQ IN NO: 9, and a full length light chain having an amino acid sequence of SEQ ID NO: 10;
(b) a full length heavy chain having an amino acid sequence of SEQ IN NO: 7, and a full length light chain having an amino acid sequence of SEQ ID NO:8; or
(c) a heavy chain variable region (VH) having an amino acid sequence of

SEQ ID NO:5, and a light chain variable region (VL) of an amino acid sequence of SEQ ID NO:4.

In another aspect of the embodiments described above, and in combination of any other aspect not inconsistent, the PD-1 axis binding antagonist is an anti-PD-L1 antibody. In one embodiment, the anti-PD-L1 antibody is selected from the group consisting of avelumab, atezolizumab and durvalumab. In another embodiment, the anti-PD-L1 antibody comprises
(a) a VH having an amino acid sequence of SEQ ID NO:14, and a VL having an amino acid sequences of SEQ ID NO:15; or
(b) a VH having an amino acid sequence of SEQ ID NO:12, and a VL having an amino acid sequence of SEQ ID NO:13.

In another aspect of the embodiments described above, and in combination of any other aspect not inconsistent, the PD-1 axis binding antagonist is administered at a dose of about 5 mg/kg, about 10 mg/kg, about 200 mg, about 240 mg, about 800 mg or about 1200 mg, and is administered about once a week, or about once every two, three, four, five weeks or six weeks; and the VEGF pathway inhibitor is administered at a dose of about 3 mg/kg, about 5 mg/kg, about 3 mg, or about 5 mg and is administered twice daily.

In another aspect of the embodiments described above, and in combination of any other aspect not inconsistent, the cancer is advanced or metastatic solid tumor.

In another aspect of the embodiments described above, and in combination of any other aspect not inconsistent, the cancer is bladder cancer, breast cancer, clear cell kidney cancer, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), small lymphocytic lymphoma (SLL), endometrial cancer, B-cell acute lymphoblastic leukemia, colorectal cancer, glioblastoma, cervical cancer, penile cancer, or non-melanoma skin cancer.

In any of the preceding examples involving an increased expression level of UTS2 in the sample (e.g., a tissue sample (e.g., a tumor tissue sample)) obtained from a patient, expression of UTS2 has been determined to have increased by 1% or more (e.g., 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, or 50% or more, preferably 5% or more) relative to a reference level of UTS2. Optionally, the reference level of UTS2 is an average UTS2 expression level (e.g. median) across samples from multiple patients. Optionally, an increased expression level is an expression level greater than the median expression level across samples from multiple patients.

In any of the preceding examples involving an increased expression level of at least 1, 2, 3, 4, 5, 6, 7, 8, 10, 15, 20, 25, or all 26 of the genes CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCL5, XCL2, CST7, GFI1, KCNA3, PSTPIP1 in the sample (e.g., a tissue sample (e.g., a tumor tissue sample)) obtained from a patient, expression the at least 1, 2, 3, 4, 5, 6, 7, 8, 10, 15, 20, 25, or all 26 of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCL5, XCL2, CST7, GFI1, KCNA3, PSTPIP1 has been determined to have increased by 1% or more (e.g., 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, or 50% or more, preferably 5% or more) relative to a reference level of the respective gene(s). Optionally, the reference level of an average expression level (e.g. median) of the respective gene(s) across samples from multiple patients. Optionally, an increased expression level is an expression level greater than the median expression level of the gene across samples from multiple patients.

In any of the preceding examples involving an increased expression level of DUX4 gene or the DUX4 gene signature in the sample (e.g., a tissue sample (e.g., a tumor tissue sample)) obtained from a patient, expression of DUX4 or the DUX4 gene signature has been determined to have increased by 1% or more (e.g., 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, or 50% or more, preferably 5% or more) relative to a reference level of DUX4. Optionally, the reference level of DUX4 is an average DUX4 expression level (e.g. median) across samples from multiple patients. Optionally, an increased expression level is an expression level greater than the median expression level across samples from multiple patients.

In any of the preceding examples involving an increased expression level of at least 1, 2, 3, 4, 5, 6, 7, 8, 10, 15, 20, 25, or all 26 of the genes NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB in the sample (e.g., a tissue sample (e.g., a tumor tissue sample)) obtained from a patient, expression the at least 1, 2, 3, 4, 5, 6, 7, 8, 10, 15, 20, 25, or all 26 of NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJA5, ENPP2, CASQ2, PTPRB, TBX2, ATP1A2, CD34, HEY2, EDNRB has been determined to have increased by 1% or more (e.g., 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, or 50% or more, preferably 5% or more) relative to a reference level of the respective gene(s). Optionally, the reference level of an average expression level (e.g. median) of the respective gene(s) across samples from multiple patients. Optionally, an increased expression level is an expression level greater than the median expression level of the gene across samples from multiple patients.

In any of the preceding methods, the presence and/or expression level (amount) of a biomarker (e.g., UTS2, CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1, DUX4, NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJA5, ENPP2, CASQ2, PTPRB, TBX2, ATP1A2, CD34, HEY2, EDNRB, CD8) may be a nucleic acid expression level. In some instances, the nucleic acid expression level is determined using qPCR, rtPCR, RNA-Seq, multiplex qPCR or RT-qPCR, microarray analysis, SAGE, MassARRAY technique, or in situ hybridization (e.g., FISH).

In some instances, the expression level of a biomarker is determined in tumor cells, tumor infiltrating immune cells, stromal cells, or combinations thereof.

In a particular instance, the expression level of a biomarker (e.g., UTS2, CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1, DUX4, NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJA5, ENPP2, CASQ2, PTPRB, TBX2, ATP1A2, CD34, HEY2, EDNRB, CD8) is an mRNA expression level. Methods for the evaluation of mRNAs in cells are well known and include, for example, RNA-Seq (e.g., whole transcriptome shotgun sequencing) using next generation sequencing techniques, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined. Optional methods include protocols that examine or detect mRNAs, such as target mRNAs, in a tissue or cell sample by microarray technologies. Using nucleic acid microarrays test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment including a PD-1 axis binding antagonist (and optionally which further comprises a VEGF pathway inhibitor) may be arrayed on a solid support. Hybridization of a labeled probe with a particular array member indicates that the sample from which the probe was derived expresses that gene.

In any of the preceding methods, the presence and/or expression level (amount) of a biomarker (e.g., UTS2, CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1, DUX4, NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB, CD8) is measured by determining protein expression levels of the biomarker. In certain instances, the method comprises contacting the biological sample with antibodies that specifically bind to a biomarker described herein under conditions permissive for binding of the biomarker, and detecting whether a complex is formed between the antibodies and biomarker. Such method may be an in vitro or in vivo method. Any method of measuring protein expression levels known in the art may be used. For example, in some instances, a protein expression level of a biomarker is determined using a method selected from the group consisting of flow cytometry (e.g., fluorescence-activated cell sorting (FACS)), Western blot, ELISA, ELIFA, immunoprecipitation, immunohistochemistry (IHC), immunofluorescence, radioimmunoassay, dot blotting, immunodetection methods, HPLC, surface plasmon resonance, optical spectroscopy, mass spectrometry, and HPLC. In some instances, the protein expression level of the biomarkers is determined in tumor cells.

In certain instances, a reference level, reference sample, reference cell, reference tissue, control sample, control cell, or control tissue is a single sample or a combination of multiple samples from the same subject or individual that are obtained at one or more different time points than when the test sample is obtained. For example, a reference level, reference sample, reference cell, reference tissue, control sample, control cell, or control tissue is obtained at an earlier time point from the same subject or individual than when the test sample is obtained. Such reference level, reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference level, reference sample, reference cell, reference tissue, control sample, control cell, or control tissue is a combination of multiple samples from one or more healthy individuals who are not the patient.

In certain embodiments, a reference level, reference sample, reference cell, reference tissue, control sample, control cell, or control tissue is a combination of multiple samples from one or more individuals with a disease or disorder (e.g., cancer) who are not the patient/individual. In certain embodiments, a reference level, reference sample, reference cell, reference tissue, control sample, control cell, or control tissue is a combination of multiple samples from multiple individuals with a disease or disorder (e.g., cancer), wherein the multiple individuals are the patient/individual plus one or more other more patients/individuals with the disease or disorder (e.g., cancer).

In certain embodiments, a reference level, reference sample, reference cell, reference tissue, control sample, control cell, or control tissue is pooled RNA samples from normal tissues or pooled plasma or serum samples from one or more individuals who are not the patient.

In certain embodiments, a reference level, reference sample, reference cell, reference tissue, control sample, control cell, or control tissue is pooled RNA samples from tumor tissues or pooled plasma or serum samples from one or more individuals with a disease or disorder (e.g., cancer) who are not the patient. In certain embodiments, a reference level, reference sample, reference cell, reference tissue, control sample, control cell, or control tissue is pooled RNA samples from tumor tissues or pooled plasma or serum samples from multiple individuals with a disease or disorder (e.g., cancer), wherein the multiple individuals are the patient/individual plus one or more other more patients/individuals with the disease or disorder (e.g., cancer).

In certain embodiments, the reference level is the median level of expression of a biomarker across a set of samples (e.g., a set of tissue samples (e.g., a set of tumor tissue samples)). In certain embodiments, the reference level is the median level of expression of a biomarker across a population of patients having a particular disease or disorder (e.g., a proliferative cell disorder (e.g., a cancer)).

In some embodiments provided herein involving a combination of more than one sample (e.g. an average level of a biomarker across multiple samples), there are at least 3, 5, 10, 15, 20, 25, 30, 40, 50, 75, 100, 150, 200, 250, or 500 samples in the combined sample set. In some embodiments provided herein involving a combination of samples from more than one individuals/patients (e.g. an average level of a biomarker across samples from multiple individuals/patients), samples are from at least 3, 5, 10, 15, 20, 25, 30, 40, 50, 75, 100, 150, 200, 250, or 500 individuals/patients.

In some embodiments of any of the methods provide herein involving elevated or increased expression of a biomarker, elevated or increased expression refers to an overall increase of any of 10% or greater, 20% or greater, 30% or greater, 40% or greater, 50% or greater, 60% or greater, 70% or greater, 80% or greater, 90% or greater, 95% or greater, 96% or greater, 97% or greater, 98% or greater, 99% or greater, in the level of biomarker (e.g., protein or nucleic acid (e.g., gene or mRNA)), detected by standard art-known methods such as those described herein, as compared to a reference level, reference sample, reference cell, reference tissue, control sample, control cell, or control tissue. In certain embodiments, the elevated or increased expression refers to the increase in expression level (amount) of a biomarker in the sample, wherein the increase is at least any of 1.5 times,1.75 times, 2 times, 3 times, 4 times, 5 times, 6 times, 7 times, 8 times, 9 times, 10 times, 25 times, 50 times, 75 times, or 100 times the expression level (amount) of the respective biomarker in a reference level, reference sample, reference cell, reference tissue, control sample, control cell, or control tissue. In some embodiments, elevated expression refers to an overall increase of greater than 1.5-fold, 1.75-fold, 2-fold, 2.25-fold, 2.5-fold, 2.75-fold, 3.0-fold, or 3.25-fold as compared to a reference sample, reference cell, reference tissue, control sample, control cell, control tissue, or internal control (e.g., housekeeping gene).

In some embodiments of any of the methods involving reduced expression of a biomarker, reduced or decreased expression refers to an overall reduction of any of 10% or greater, 20% or greater, 30% or greater, 40% or greater, 50% or greater, 60% or greater, 70% or greater, 80% or greater, 90% or greater, 95% or greater, 96% or greater, 97% or greater, 98% or greater, 99% or greater, in the level of biomarker (e.g., protein or nucleic acid (e.g., gene or mRNA)), detected by standard art known methods such as those described herein, as compared to a reference level, reference sample, reference cell, reference tissue, control sample, control cell, or control tissue. In certain embodiments, reduced expression refers to the decrease in expression level (amount) of a biomarker in the sample wherein the decrease is at least any of 0.9 times, 0.8 times, 0.7 times, 0.6 times, 0.5 times, 0.4 times, 0.3 times, 0.2 times, 0.1 times, 0.05 times, or 0.01 times the expression level (amount) of the respective biomarker in a reference level, reference sample, reference cell, reference tissue, control sample, control cell, control tissue, or internal control (e.g., housekeeping gene).

In some embodiments of any of the methods provided herein involving increased or high expression of a biomarker in a sample (e.g. UTS2 gene), increased or high expression refers to above 0.5, 0.4, 0.3, 0.2, 0.1, 0.05, 0.04, 0.03, 0.02, or 0.01 log2 Transcripts Per Million (TPM) in the sample.

In some embodiments of any of the methods provided herein involving reduced or low expression of a biomarker in a sample (e.g. DUX4 gene or gene signature), reduced or low expression refers to below 0.5, 0.4, 0.3, 0.2, 0.1, 0.05, 0.04, 0.03, 0.02, or 0.01 log2 Transcripts Per Million (TPM) in the sample.

### BRIEF DESCRIPTION OF THE FIGURES/DRAWINGS

FIG. 1 depicts progression free survival data of patients with CD163L1 mutations from phase 3 clinical trial of avelumab with axitinib versus sunitinib in advanced renal cell cancer. The X-axis shows months and the Y-axis shows survival probability. Data for patients with a mutation and on the avelumab+axitinib arm are shown in the line labeled with a solid circle (25 patients); data for patients with the wild-type gene and on the avelumab+axitinib arm are shown in the line labeled with an empty circle (346 patients); data for patients with a mutation and on the sunitinib arm are shown in the line labeled with an solid triangle (22 patients); data for patients with a the wild-type gene and on the sunitinib arm are shown in the line labeled with an empty triangle (355 patients).

FIG. 2 depicts progression free survival data of patients with DNMT1 mutations from phase 3 clinical trial of avelumab with axitinib versus sunitinib in advanced renal cell cancer. The X-axis shows months and the Y-axis shows survival probability. Data for patients with a mutation and on the avelumab+axitinib arm are shown in the line labeled with a solid circle (21 patients); data for patients with the wild-type gene and on the avelumab+axitinib arm are shown in the line labeled with an empty circle (350 patients); data for patients with a mutation and on the sunitinib arm are shown in the line labeled with an solid triangle (16 patients); data for patients with a the wild-type gene and on the sunitinib arm are shown in the line labeled with an empty triangle (361 patients).

FIG. 3 depicts progression free survival data of patients with MC1R mutations from phase 3 clinical trial of avelumab with axitinib versus sunitinib in advanced renal cell cancer. The X-axis shows months and the Y-axis shows survival probability. Data for patients with a mutation and on the avelumab+axitinib arm are shown in the line labeled with a solid circle (20 patients); data for patients with the wild-type gene and on the avelumab+axitinib arm are shown in the line labeled with an empty circle (351 patients); data for patients with a mutation and on the sunitinib arm are shown in the line labeled with an solid triangle (15 patients); data for patients with a the wild-type gene and on the sunitinib arm are shown in the line labeled with an empty triangle (362 patients).

FIG. 4 depicts progression free survival data of patients with ABCA1 mutations from phase 3 clinical trial of avelumab with axitinib versus sunitinib in advanced renal cell cancer. The X-axis shows months and the Y-axis shows survival probability. Data for patients with a mutation and on the avelumab+axitinib arm are shown in the line labeled with a solid circle (27 patients); data for patients with the wild-type gene and on the avelumab+axitinib arm are shown in the line labeled with an empty circle (344 patients); data for patients with a mutation and on the sunitinib arm are shown in the line labeled with an solid triangle (23 patients); data for patients with a the wild-type gene and on the sunitinib arm are shown in the line labeled with an empty triangle (354 patients).

FIG. 5 depicts progression free survival data of patients with FOXO1 mutations from phase 3 clinical trial of avelumab with axitinib versus sunitinib in advanced renal cell cancer. The X-axis shows months and the Y-axis shows survival probability. Data for patients with a mutation and on the avelumab+axitinib arm are shown in the line labeled with a solid circle (32 patients); data for patients with the wild-type gene and on the avelumab+axitinib arm are shown in the line labeled with an empty circle (339 patients); data for patients with a mutation and on the sunitinib arm are shown in the line labeled with an solid triangle (25 patients); data for patients with a the wild-type gene and on the sunitinib arm are shown in the line labeled with an empty triangle (352 patients).

FIG. 6 depicts progression free survival data of patients with IL16 mutations from phase 3 clinical trial of avelumab with axitinib versus sunitinib in advanced renal cell cancer. The X-axis shows months and the Y-axis shows survival probability. Data for patients with a mutation and on the avelumab+axitinib arm are shown in the line labeled with a solid circle (63 patients); data for patients with the wild-type gene and on the avelumab+axitinib arm are shown in the line labeled with an empty circle (308 patients); data for patients with a mutation and on the sunitinib arm are shown in the line labeled with an solid triangle (53 patients); data for patients with a the wild-type gene and on the sunitinib arm are shown in the line labeled with an empty triangle (324 patients).

FIG. 7 depicts progression free survival data of patients with MYH7B mutations from phase 3 clinical trial of avelumab with axitinib versus sunitinib in advanced renal cell cancer. The X-axis shows months and the Y-axis shows survival probability. Data for patients with a mutation and on the avelumab+axitinib arm are shown in the line labeled with a solid circle (42 patients); data for patients with the wild-type gene and on the avelumab+axitinib arm are shown in the line labeled with an empty circle (329 patients); data for patients with a mutation and on the sunitinib arm are shown in the line labeled with an solid triangle (38 patients); data for patients with a the wild-type gene and on the sunitinib arm are shown in the line labeled with an empty triangle (339 patients).

FIG. 8 depicts progression free survival data of patients with STAB2 mutations from phase 3 clinical trial of avelumab with axitinib versus sunitinib in advanced renal cell cancer. The X-axis shows months and the Y-axis shows survival probability. Data for patients with a mutation and on the avelumab+axitinib arm are shown in the line labeled with a solid circle (30 patients); data for patients with the wild-type gene and on the avelumab+axitinib arm are shown in the line labeled with an empty circle (341 patients); data for patients with a mutation and on the sunitinib arm are shown in the line labeled with an solid triangle (37 patients); data for patients with a the wild-type gene and on the sunitinib arm are shown in the line labeled with an empty triangle (340 patients).

FIG. 9 depicts progression free survival data of patients with LOC728763 mutations from phase 3 clinical trial of avelumab with axitinib versus sunitinib in advanced renal cell cancer. The X-axis shows months and the Y-axis shows survival probability. Data for patients with a mutation and on the avelumab+axitinib arm are shown in the line labeled with a solid circle (39 patients); data for patients with the wild-type gene and on the avelumab+axitinib arm are shown in the line labeled with an empty circle (332 patients); data for patients with a mutation and on the sunitinib arm are shown in the line labeled with an solid triangle (27 patients); data for patients with a the wild-type gene and on the sunitinib arm are shown in the line labeled with an empty triangle (350 patients).

FIG. 10 depicts progression free survival data of patients with SPATA31C2 mutations from phase 3 clinical trial of avelumab with axitinib versus sunitinib in advanced renal cell cancer. The X-axis shows months and the Y-axis shows survival probability. Data for patients with a mutation and on the avelumab+axitinib arm are shown in the line labeled with a solid circle (55 patients); data for patients with the wild-type gene and on the avelumab+axitinib arm are shown in the line labeled with an empty circle (316 patients); data for patients with a mutation and on the sunitinib arm are shown in the line labeled with an solid triangle (54 patients); data for patients with a the wild-type gene and on the sunitinib arm are shown in the line labeled with an empty triangle (323 patients).

FIG. 11 depicts progression free survival data of patients with PTEN mutations from phase 3 clinical trial of avelumab with axitinib versus sunitinib in advanced renal cell cancer. The X-axis shows months and the Y-axis shows survival probability. Data for patients with a mutation and on the avelumab+axitinib arm are shown in the line labeled with a solid circle (23 patients); data for patients with the wild-type gene and on the avelumab+axitinib arm are shown in the line labeled with an empty circle (348 patients); data for patients with a mutation and on the sunitinib arm are shown in the line labeled with an solid triangle (34 patients); data for patients with a the wild-type gene and on the sunitinib arm are shown in the line labeled with an empty triangle (343 patients).

FIG. 12 depicts progression free survival data of patients with mutations in two genes among CD163L1, DNMT1 and MC1R, from phase 3 clinical trial of avelumab with axitinib versus sunitinib in advanced renal cell cancer. The X-axis shows months and the Y-axis shows survival probability. Data for patients with a single mutation and on the avelumab+axitinib arm are shown in the line labeled with a solid circle (56 patients); data for patients with a double mutation and on the avelumab+axitinib arm are shown in the line labeled with a solid diamond (5 patients); data for patients with the wild-type gene and on the avelumab+axitinib arm are shown in the line labeled with an empty circle (310 patients); data for patients with a single mutation and on the sunitinib arm are shown in the line labeled with an solid triangle (49 patients); data for patients with a double mutation and on the sunitinib arm are shown in the line labeled with an empty diamond (2 patients); data for patients with a the wild-type gene and on the sunitinib arm are shown in the line labeled with an empty triangle (326 patients).

FIG. 13A depicts progression free survival data of patients from phase 3 clinical trial treated with the combination of avelumab and axitinib with either a high number of CD8 positive cells (greater than or equal to median value)(line labeled with a solid circle) or a low number of CD8 positive cells (less than median value) (line labeled with an empty circle) at the invasive margin. The X-axis depicts months, and the Y-axis depicts progression-free survival percentage. The FIG. 13B depicts progression free survival data of patients from phase 3 clinical trial treated with sunitinib with either a high number of CD8 positive cells (greater than or equal to median value) (line labeled with a solid triangle) or a low number of CD8 positive cells (less than median value) (line labeled with an empty triangle) at the invasive margin. The X-axis depicts months, and the Y-axis depicts progression-free survival percentage.

FIG. 14A depicts progression free survival data of patients from phase 3 clinical trial treated with the combination of avelumab and axitinib with either a high expression of the JAVELIN Renal 101 Immuno Signature (greater than or equal to median value) (line labeled with a solid circle) or a low expression of the JAVELIN Renal 101 Immuno Signature (less than median value) (line labeled with an empty circle). The X-axis depicts months, and the Y-axis depicts progression-free survival percentage. FIG. 14B depicts progression free survival data of patients from phase 3 clinical trial treated with sunitinib with either a high expression of the JAVELIN Renal 101 Immuno Signature (greater than or equal to median value) or a low expression of the JAVELIN Renal 101 Immuno Signature (less than median value). The X-axis depicts months, and the Y-axis depicts progression-free survival percentage.

FIG. 15 depicts progression free survival data of patients from phase 1 b clinical trial treated with the combination of avelumab and axitinib with either a high expression of the JAVELIN Renal 101 Immuno Signature (greater than or equal to median value) (line labeled with a solid circle) or a low expression of the JAVELIN Renal 101 Immuno Signature (less than median value) (line labeled with an empty circle). The X-axis depicts months, and the Y-axis depicts progression-free survival percentage.

FIG. 16A depicts progression free survival data of patients from phase 3 clinical trial treated with sunitinib with either a high expression of the JAVELIN Renal 101 Angiogenesis Signature (greater than median value) (line labeled with a solid circle) or a low expression of the JAVELIN Renal 101 Angiogenesis Signature (less than median value) (line labeled with an empty circle). The X-axis shows months and the Y-axis shows survival probability. FIG. 16B depicts progression free survival data of patients from phase 3 clinical trial treated with the combination of avelumab and axitinib with either a high expression of the JAVELIN Renal 101 Angiogenesis Signature (greater than median value) (line labeled with a solid circle) or a low expression of the JAVELIN Renal 101 Angiogenesis Signature (less than median value) (line labeled with an empty circle). The X-axis shows months and the Y-axis shows survival probability.

FIG. 17 depicts Log2 Hazard Ratio of patients from phase 3 clinical trial treated with the combination of avelumab and axitinib or with sunitinib with either a high expression or low expression of various signatures as shown, including the JAVELIN Renal 101 Signature, the JAVELIN Renal 101 Angiogenesis Signature, the IMmotion 150 T_{effector} signature, the IMmotion Angiogeneis signature, the IMmotion 150 myeloid inflamed signature/T_{effector}^{high} signatures, the tumor mutational burden (TMB)/myeloid inflamed^{high} gene signatures, and nonsynonymous coding signature.

FIG. 18 depicts progression free survival data of patients from phase 3 clinical trial treated with the combination of avelumab and axitinib or with sunitinib with either a high expression of the DUX4 gene signature (greater than median value) (avelumab and axitinib: line labeled with a solid circle; sunitinib: line labeled with a solid triangle) or a low expression of the DUX4 gene signature (less than median value) (avelumab and axitinib: line labeled with an empty circle; sunitinib: line labeled with an empty triangle). The X-axis shows months and the Y-axis shows survival probability (progression free survival).

FIG. 19 depicts Log2 Hazard Ratio of patients from phase 3 clinical trial treated with the combination of avelumab and axitinib or with sunitinib and having either wild type or mutations in the genes CD163L1, DNMT1, IL16, MC1R, PTEN, ABCA1, FOXO1, LOC728763, MYH7B, SPATA31C2, and STAB2.

FIG. 20A depicts progression free survival data of patients from the phase 3 clinical trial treated with the combination of avelumab and axitinib having mutations in i) none of the genes CD163L1, DNMT1, IL16, MC1R, ABCA1, FOXO1, LOC728763, MYH7B, SPATA31C2, and STAB2 (e.g. all of these genes wild-type) (line labeled with an empty circle); ii) one of the genes CD163L1, DNMT1, IL16, MC1R, ABCA1, FOXO1, LOC728763, MYH7B, SPATA31C2, and STAB2 (line labeled with a solid circle); or iii) two or more of the genes CD163L1, DNMT1, IL16, MC1R, ABCA1, FOXO1, LOC728763, MYH7B, SPATA31C2, and STAB2 (line labeled with a solid diamond). The X-axis shows months and the Y-axis shows survival probability (progression free survival).

FIG. 20B depicts progression free survival data of patients from the phase 3 clinical trial treated with sunitinib having mutations in i) none of the genes CD163L1, DNMT1, IL16, MC1R, ABCA1, FOXO1, LOC728763, MYH7B, SPATA31C2, and STAB2 (e.g. all of these genes wild-type)(line labeled with an empty triangle); ii) one of the genes CD163L1, DNMT1, IL16, MC1R, ABCA1, FOXO1, LOC728763, MYH7B, SPATA31C2, and STAB2 (line labeled with a solid triangle); or iii) two or more of the genes CD163L1, DNMT1, IL16, MC1R, ABCA1, FOXO1, LOC728763, MYH7B, SPATA31C2, and STAB2 (line labeled with an empty diamond). The X-axis shows months and the Y-axis shows survival probability (progression free survival).

FIG. 21A depicts progression free survival data of patients from the phase 3 clinical trial treated with the combination of avelumab and axitinib having either a high expression of the UTS2 gene (greater than median value) (line labeled with a solid circle) or a low expression of the UTS2 gene (less than median value) (line labeled with an empty circle). The X-axis shows months and the Y-axis shows survival probability (progression free survival). FIG. 21B depicts progression free survival data of patients from the phase 3 clinical trial treated with sunitinib having either a high expression of the UTS2 gene (greater than median value) (line labeled with a solid triangle) or a low expression of the UTS2 gene (less than median value) (line labeled with an empty triangle). The X-axis shows months and the Y-axis shows survival probability (progression free survival).

### DETAILED DESCRIPTION

### I. DEFINITIONS

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“About” when used to modify a numerically defined parameter (e.g., the dose of a PD-1 axis binding antagonist or VEGF pathway inhibitor, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg.

As used herein, including the appended claims, the singular forms of words such as “a,” “an,” and “the,” include their corresponding plural references unless the context clearly dictates otherwise.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv), single chain (scFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen (e.g., PD-1 or PD-L1). Antigen binding functions of an antibody can be performed by fragments of an intact antibody. Examples of binding fragments encompassed within the term “antigen binding fragment” of an antibody include Fab; Fab′; F(ab′)2; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody, an antibody conjugate, or a polypeptide that “preferentially binds” or “specifically binds” (used interchangeably herein) to a target (e.g., PD-1 or PD-L1 protein) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a PD-L1 epitope is an antibody that binds this epitope with greater affinity, avidity, more readily, and/or with greater duration than it binds to other PD-L1 epitopes or non-PD-L1 epitopes. It is also understood that by reading this definition, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chains is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The term “biomarker” as used herein refers to an indicator molecule or set of molecules (e.g., predictive, diagnostic, and/or prognostic indicator), which can be detected in a sample. The biomarker may be a predictive biomarker and serve as an indicator of the likelihood of sensitivity or benefit of a patient having a particular disease or disorder (e.g., a proliferative cell disorder (e.g., cancer)) to a particular treatment (e.g. treatment with one or both of a PD-1 axis binding antagonist and a VEGF pathway inhibitor). Biomarkers include, but are not limited to, polynucleotides (e.g., DNA and/or RNA (e.g., mRNA)), polynucleotide copy number alterations (e.g., DNA copy numbers), polynucleotide sequence alterations (e.g. gene mutations or gene variants), polypeptides, polypeptide and polynucleotide modifications (e.g., post-translational modifications), carbohydrates, and/or glycolipid-based molecular markers. In some embodiments, a biomarker is a gene.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Another particular example of cancer includes renal cell carcinoma.

“Biotherapeutic agent” means a biological molecule, such as an antibody or fusion protein, that blocks ligand/receptor signaling in any biological pathway that supports tumor maintenance and/or growth or suppresses the anti-tumor immune response.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods of the present invention include cytostatic and/or cytotoxic agents.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a PD-1 axis binding antagonist that consists essentially of a recited amino acid sequence may also include one or more amino acids, including substitutions of one or more amino acid residues, which do not materially affect the properties of the binding compound.

“Genetic mutation”, or “genetic alteration”, as used here in, refer to a germline, somatic or recombinant mutation of a wild type gene, including substitution, insertion, and deletion of one or more nucleotides in the gene's coding or non-coding sequence.

“A protein altering mutation” as used herein refers to a genetic mutation that (a) results in a change in the amino acid sequence of the corresponding protein; or (b) otherwise results in a disruption of the expression, or function of the protein which the gene encodes. Examples of a protein altering genetic mutation includes but is not limited to disruptive inframe deletion, disruptive inframe insertion, frameshift variant, inframe deletion, inframe insertion, initiator codon variant, intron variant, missense variant, non-canonical start codon, splice acceptor variant, splice donor variant, splice region variant, start lost, stop gained, stop lost, and stop retained variant.

“Expression level”, “level of expression” and the like refers to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a posttranslational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression”, “increased expression level”, “increased levels”, “elevated expression”, “elevated expression levels”, or “elevated levels” refers to an increased expression or increased levels of a biomarker in an individual relative to a control, such as an individual or individuals who do not have the disease or disorder (e.g., cancer), an internal control (e.g., a housekeeping biomarker), or a median expression level of the biomarker in samples from a group/population of patients.

“Decreased expression”, “decreased expression level”, “decreased levels”, “reduced expression”, “reduced expression levels”, or “reduced levels” refers to a decrease expression or decreased levels of a biomarker in an individual relative to a control, such as an individual or individuals who do not have the disease or disorder (e.g., cancer), an internal control (e.g., a housekeeping biomarker), or a median expression level of the biomarker in samples from a group/population of patients. In some embodiments, reduced expression is little or no expression.

“Housekeeping gene” refers herein to a gene or group of genes that encode proteins whose activities are essential for the maintenance of cell function and which are typically similarly present in all cell types. In some embodiments, the housekeeping gene can be beta actin (ACTB), glyceraldehyde 3-phosphate dehydrogenase (GAPDH), phosphoglycerate kinase 1 (PGK1), heterogenous nuclear ribonucleoprotein L (HNRNPL), poly-binding protein 1 (PCBP1), or retention in endoplasmic reticulum sorting receptor 1 (RER1).

“CD163L1” as used herein, refers to the CD163 molecule like 1 gene or the protein encoded by the gene. This gene encodes a member of the scavenger receptor cysteine-rich (SRCR) superfamily. Members of this family are secreted or membrane-anchored proteins mainly found in cells associated with the immune system. The SRCR family is defined by a 100-110 amino acid SRCR domain, which may mediate protein-protein interaction and ligand binding. Broad expression of the SRCR family is found in spleen (RPKM 7.7), small intestine (RPKM 4.5) and 14 other tissues. Exemplary mutations in CD163L1 useful in the embodiments disclosed herein are provided in Table 1.1 below. All mutations therein were reported based on genetic analysis of patients' tumor samples in Example 3.

TABLE 1.1
Exemplary CD163L mutations

| Gene | Position | Mutation | # | Percent |
|---|---|---|---|---|
| CD163L1 | 191 | V191M | 1 | 0.13 |
| CD163L1 | 215 | R215C | 1 | 0.13 |
| CD163L1 | 247 | N247D | 1 | 0.13 |
| CD163L1 | 273 | G273E | 1 | 0.13 |
| CD163L1 | 274 | R274S | 1 | 0.13 |
| CD163L1 | 276 | E276Q | 1 | 0.13 |
| CD163L1 | 433 | I433R | 1 | 0.13 |
| CD163L1 | 456 | R456* | 1 | 0.13 |
| CD163L1 | 457 | T457A | 1 | 0.13 |
| CD163L1 | 487 | G487E | 1 | 0.13 |
| CD163L1 | 591 | R591L | 1 | 0.13 |
| CD163L1 | 600 | F600L | 4 | 0.53 |
| CD163L1 | 739 | V739A | 1 | 0.13 |
| CD163L1 | 749 | T749I | 1 | 0.13 |
| CD163L1 | 754 | M754V | 1 | 0.13 |
| CD163L1 | 764 | A764G | 1 | 0.13 |
| CD163L1 | 860 | E860K | 5 | 0.67 |
| CD163L1 | 862 | F862I | 1 | 0.13 |
| CD163L1 | 908 | Q908L | 1 | 0.13 |
| CD163L1 | 929 | W929* | 1 | 0.13 |
| CD163L1 | 946 | A946V | 8 | 1.07 |
| CD163L1 | 962 | V962M | 1 | 0.13 |
| CD163L1 | 1069 | D1069Y | 1 | 0.13 |
| CD163L1 | 1161 | N1161T | 1 | 0.13 |
| CD163L1 | 1166 | S1166N | 1 | 0.13 |
| CD163L1 | 1174 | T1174A | 1 | 0.13 |
| CD163L1 | 1249 | R1249H | 1 | 0.13 |
| CD163L1 | 1307 | T1307A | 2 | 0.27 |
| CD163L1 | 1356 | S1356P | 6 | 0.8 |
| CD163L1 | 1408 | F1408L | 1 | 0.13 |
| CD163L1 | 1439 | D1439N | 1 | 0.13 |
| CD163L1 | 1441 | S1441W | 1 | 0.13 |

“DNMT1” as used herein, refers to the DNMT1- DNA methyltransferase 1 gene or the protein encoded by the gene. This gene encodes an enzyme that transfers methyl groups to cytosine nucleotides of genomic DNA. This protein is the major enzyme responsible for maintaining methylation patterns following DNA replication and shows a preference for hemi-methylated DNA. Methylation of DNA is an important component of mammalian epigenetic gene regulation. Aberrant methylation patterns are found in human tumors and associated with developmental abnormalities. Exemplary mutations in DNMT1 useful in the embodiments disclosed herein are provided in Table 1.2 below. All mutations therein were reported based on genetic analysis of patients' tumor samples in Example 3.

TABLE 1.2
Exemplary DNMT1 mutations

| Gene | Position | Mutation | # | Percent |
|---|---|---|---|---|
| DNMT1 | 145 | G145V | 1 | 0.13 |
| DNMT1 | 151 | R151S | 1 | 0.13 |
| DNMT1 | 159 | Q159R. | 3 | 0.4 |
| | | Q159fs | | |
| DNMT1 | 161 | T161A | 11 | 1.47 |
| DNMT1 | 164 | R164T | 1 | 0.13 |
| DNMT1 | 308 | D308Y | 1 | 0.13 |
| DNMT1 | 326 | Q326P | 1 | 0.13 |
| DNMT1 | 355 | R355C | 1 | 0.13 |
| DNMT1 | 369 | C369Y | 1 | 0.13 |
| DNMT1 | 560 | R560C | 1 | 0.13 |
| DNMT1 | 686 | C686Y | 1 | 0.13 |
| DNMT1 | 876 | G876R | 1 | 0.13 |
| DNMT1 | 910 | V910L | 1 | 0.13 |
| DNMT1 | 911 | S911R | 1 | 0.13 |
| DNMT1 | 983 | D983E | 1 | 0.13 |
| DNMT1 | 1277 | R1277Q | 1 | 0.13 |
| DNMT1 | 1461 | R1461W | 1 | 0.13 |
| DNMT1 | 1476 | H1476Q | 8 | 1.07 |

“MC1 R” as used herein, refers to the melanocortin 1 receptor gene or the protein encoded by the gene. Exemplary mutations in MC1 R useful in the embodiments disclosed herein are provided in Table 1.3 below. All mutations therein were reported based on genetic analysis of patients' tumor samples in Example 3.

TABLE 1.3
Exemplary MC1R mutations

| Gene | Position | Mutation | # | Percent |
|---|---|---|---|---|
| MC1R | 19 | T19I | 1 | 0.13 |
| MC1R | 29 | N29fs | 4 | 0.53 |
| MC1R | 38 | V38M | 2 | 0.27 |
| MC1R | 52 | S52R | 1 | 0.13 |
| MC1R | 57 | A57T | 1 | 0.13 |
| MC1R | 67 | R67Q | 1 | 0.13 |
| MC1R | 83 | S83P | 2 | 0.27 |
| MC1R | 84 | D84E | 9 | 1.20 |
| MC1R | 95 | T95M | 1 | 0.13 |
| MC1R | 111 | A111V | 1 | 0.13 |
| MC1R | 142 | R142C | 1 | 0.13 |
| MC1R | 152 | Y152* | 2 | 0.27 |
| MC1R | 166 | A166G | 1 | 0.13 |
| MC1R | 180 | I180fs | 1 | 0.13 |
| MC1R | 213 | R213W | 1 | 0.13 |
| MC1R | 249 | I249V | 2 | 0.27 |
| MC1R | 278 | K278E | 1 | 0.13 |
| MC1R | 279 | N279K | 1 | 0.13 |
| MC1R | 287 | I287S | 1 | 0.13 |
| MC1R | 302 | S302R | 1 | 0.13 |

“ABCA1” as used herein, refers to the ATP binding cassette transporter gene or the protein encoded by the gene. Exemplary mutations in ABCA1 useful in the embodiments disclosed herein are provided in Table 1.4 below. All mutations therein were reported based on genetic analysis of patients' tumor samples in Example 3.

TABLE 1.4
Exemplary ABCA1 mutations

| Gene | Position | Mutation | # | Percent |
|---|---|---|---|---|
| ABCA1 | 85 | P85L | 2 | 0.27 |
| ABCA1 | 178 | I178T | 1 | 0.13 |
| ABCA1 | 184 | L184S | 1 | 0.13 |
| ABCA1 | 343 | A343V | 2 | 0.27 |
| ABCA1 | 374 | A374T | 1 | 0.13 |
| ABCA1 | 399 | V399A | 6 | 0.80 |
| ABCA1 | 446 | D446E | 2 | 0.27 |
| ABCA1 | 506 | N506S | 1 | 0.13 |
| ABCA1 | 532 | F532Y | 1 | 0.13 |
| ABCA1 | 546 | I546V | 1 | 0.13 |
| ABCA1 | 590 | W590L | 1 | 0.13 |
| ABCA1 | 717 | V717M | 1 | 0.13 |
| ABCA1 | 774 | T774P | 6 | 0.80 |
| ABCA1 | 776 | K776N | 2 | 0.27 |
| ABCA1 | 779 | A779T | 1 | 0.13 |
| ABCA1 | 868 | E868K | 1 | 0.13 |
| ABCA1 | 887 | C887F | 4 | 0.53 |
| ABCA1 | 899 | V899M | 1 | 0.13 |
| ABCA1 | 986 | D986N | 1 | 0.13 |
| ABCA1 | 1018 | D1018G | 1 | 0.13 |
| ABCA1 | 1041 | L1041Q | 1 | 0.13 |
| ABCA1 | 1181 | S1181F | 2 | 0.27 |
| ABCA1 | 1185 | N1185S | 1 | 0.13 |
| ABCA1 | 1325 | T1325I | 1 | 0.13 |
| ABCA1 | 1344 | R1344Q | 1 | 0.13 |
| ABCA1 | 1368 | V1368M | 1 | 0.13 |
| ABCA1 | 1575 | N1575S | 1 | 0.13 |
| ABCA1 | 1668 | V1668I | 1 | 0.13 |
| ABCA1 | 1669 | P1669fs | 1 | 0.13 |
| ABCA1 | 1680 | R1680Q | 1 | 0.13 |
| ABCA1 | 1746 | G1746E | 1 | 0.13 |
| ABCA1 | 1916 | E1916A | 1 | 0.13 |
| ABCA1 | 2074 | G2074S | 1 | 0.13 |
| ABCA1 | 2135 | F2135C | 1 | 0.13 |
| ABCA1 | 2191 | F2191C | 1 | 0.13 |

“FOXO1” as used herein, refers to the forkhead box protein O1 gene or the protein encoded by the gene. Exemplary mutations in FOXO1 useful in the embodiments disclosed herein are provided in Table 1.5 below. All mutations therein were reported based on genetic analysis of patients' tumor samples in Example 3.

TABLE 1.5
Exemplary FOXO1 mutations

| Gene | Position | Mutation | # | Percent |
|---|---|---|---|---|
| FOXO1 | 30 | P30R | 1 | 0.13 |
| FOXO1 | 60 | S60A | 1 | 0.13 |
| FOXO1 | 63 | A63T | 1 | 0.13 |
| FOXO1 | 79 | E79G | 1 | 0.13 |
| FOXO1 | 82 | D82N | 44 | 5.87 |
| FOXO1 | 84 | P84L | 4 | 0.53 |
| FOXO1 | 97 | A97V | 1 | 0.13 |
| FOXO1 | 104 | G104A | 1 | 0.13 |
| FOXO1 | 112 | G112V | 1 | 0.13 |
| FOXO1 | 125 | P125L | 1 | 0.13 |
| FOXO1 | 402 | T402M | 1 | 0.13 |
| FOXO1 | 644 | S644N | 1 | 0.13 |

“IL16” as used herein, refers to the interleukin 16 gene or the protein encoded by the gene. Exemplary mutations in IL16 useful in the embodiments disclosed herein are provided in Tables 1.6A and 1.6B below. All mutations therein were reported based on genetic analysis of patients' tumor samples in Example 3.

TABLE 1.6A
Exemplary IL16 Mutations

| # | Mutation |
|---|---|
| 1 | S338N |
| 1 | S471C |
| 1 | S624F |
| 1 | G1131E |
| 1 | S340L |
| 1 | G484R |
| 1 | T386M |
| 1 | E459K |
| 1 | G885R |
| 1 | A764S |
| 1 | R882W |
| 1 | Q1321* |
| 1 | R550W |
| 1 | G1060R |
| 1 | E1095K |
| 1 | H468N |
| 1 | G1060R |
| 1 | E459K |
| 1 | L1128M |
| 1 | P936fs |
| 2 | R882W |
| 1 | S624F |
| 1 | A78T |
| 1 | A1132E |
| 1 | D1068V |
| 1 | S338N |
| 1 | R882W |

TABLE 1.6B
Exemplary IL16 (isoform) Mutations

| # | Mutation |
|---|---|
| 92 | P9L |

“MYH7B” as used herein, refers to the human gene that encodes the myosin heavy chain 7B protein in human, or the protein encoded by the gene. Exemplary mutations in MYH7B useful in the embodiments disclosed herein are provided in Tables 1.7 below. All mutations therein were reported based on genetic analysis of patients' tumor samples in Example 3.

TABLE 1.7
Exemplary MYH7B Mutations

| Gene | Position | Mutation | # | Percent |
|---|---|---|---|---|
| MYH7B | 13 | P13S | 2 | 0.27 |
| MYH7B | 108 | K108N | 7 | 0.93 |
| MYH7B | 109 | V109M | 1 | 0.13 |
| MYH7B | 246 | P246L | 5 | 0.67 |
| MYH7B | 294 | R294C | 1 | 0.13 |
| MYH7B | 339 | K339M | 1 | 0.13 |
| MYH7B | 372 | E372G | 8 | 1.07 |
| MYH7B | 390 | E390* | 1 | 0.13 |
| MYH7B | 407 | M407V | 1 | 0.13 |
| MYH7B | 414 | R414W | 2 | 0.27 |
| MYH7B | 450 | R450H | 1 | 0.13 |
| MYH7B | 489 | N489S | 1 | 0.13 |
| MYH7B | 504 | V504G | 1 | 0.13 |
| MYH7B | 538 | M538fs, M538R | 3 | 0.40 |
| MYH7B | 617 | R617C, R617H | 2 | 0.27 |
| MYH7B | 649 | E649G | 1 | 0.13 |
| MYH7B | 821 | V821I | 1 | 0.13 |
| MYH7B | 843 | G843S | 1 | 0.13 |
| MYH7B | 844 | R844C | 1 | 0.13 |
| MYH7B | 865 | W865R | 1 | 0.13 |
| MYH7B | 909 | R909Q | 3 | 0.40 |
| MYH7B | 919 | R919C | 1 | 0.13 |
| MYH7B | 1006 | L1006Q | 1 | 0.13 |
| MYH7B | 1035 | D1035G | 1 | 0.13 |
| MYH7B | 1057 | G1057S | 2 | 0.27 |
| MYH7B | 1068 | A1068V | 1 | 0.13 |
| MYH7B | 1091 | K1091R | 2 | 0.27 |
| MYH7B | 1099 | R1099Q | 1 | 0.13 |
| MYH7B | 1128 | K1128* | 1 | 0.13 |
| MYH7B | 1169 | Glu1169_Glu1172del | 1 | 0.13 |
| MYH7B | 1189 | A1189V | 1 | 0.13 |
| MYH7B | 1190 | R1190W | 1 | 0.13 |
| MYH7B | 1198 | R1198W | 1 | 0.13 |
| MYH7B | 1221 | G1221R | 1 | 0.13 |
| MYH7B | 1317 | R1317Q | 1 | 0.13 |
| MYH7B | 1338 | R1338C | 1 | 0.13 |
| MYH7B | 1382 | V1382M | 1 | 0.13 |
| MYH7B | 1388 | D1388N | 1 | 0.13 |
| MYH7B | 1488 | A1488V | 1 | 0.13 |
| MYH7B | 1524 | R1524H | 1 | 0.13 |
| MYH7B | 1534 | R1534Q | 1 | 0.13 |
| MYH7B | 1538 | E1538K | 1 | 0.13 |
| MYH7B | 1543 | A1543T | 1 | 0.13 |
| MYH7B | 1549 | R1549Q | 1 | 0.13 |
| MYH7B | 1611 | Q1611K | 1 | 0.13 |
| MYH7B | 1666 | K1666M | 3 | 0.40 |
| MYH7B | 1669 | G1669S | 1 | 0.13 |
| MYH7B | 1681 | A1681V | 1 | 0.13 |
| MYH7B | 1692 | T1692M | 1 | 0.13 |
| MYH7B | 1700 | K1700fs | 1 | 0.13 |
| MYH7B | 1789 | E1789fs | 1 | 0.13 |
| MYH7B | 1831 | M1831K | 1 | 0.13 |
| MYH7B | 1836 | E1836Q | 1 | 0.13 |
| MYH7B | 1865 | E1865K | 1 | 0.13 |
| MYH7B | 1878 | E1878Q | 1 | 0.13 |
| MYH7B | 1889 | V1889L | 1 | 0.13 |
| MYH7B | 1907 | R1907G | 1 | 0.13 |
| MYH7B | 1922 | S1922R | 1 | 0.13 |
| MYH7B | 1932 | E1932K | 1 | 0.13 |
| MYH7B | 1967 | N1967del | 1 | 0.13 |
| MYH7B | 1974 | R1974W | 1 | 0.13 |

“STAB2” as used herein, refers to a human gene that encodes the stabilin-2 protein in human, or the protein encoded by the gene. Exemplary mutations in STAB2 useful in the embodiments disclosed herein are provided in Tables 1.8 below. All mutations therein were reported based on genetic analysis of patients' tumor samples in Example 3.

TABLE 1.8
Exemplary STAB2 Mutations

| Gene | Position | Mutation | # | Percent |
|---|---|---|---|---|
| STAB2 | 44 | R44* | 1 | 0.13 |
| STAB2 | 96 | L96F | 2 | 0.27 |
| STAB2 | 97 | Q97P | 1 | 0.13 |
| STAB2 | 104 | R104H | 1 | 0.13 |
| STAB2 | 219 | K219Q | 2 | 0.27 |
| STAB2 | 250 | A250V | 1 | 0.13 |
| STAB2 | 325 | I325T | 1 | 0.13 |
| STAB2 | 387 | F387Y | 3 | 0.40 |
| STAB2 | 491 | I491V | 1 | 0.13 |
| STAB2 | 559 | N559K | 1 | 0.13 |
| STAB2 | 574 | L574R | 1 | 0.13 |
| STAB2 | 587 | V587I | 1 | 0.13 |
| STAB2 | 610 | S610N | 1 | 0.13 |
| STAB2 | 636 | I636M | 1 | 0.13 |
| STAB2 | 644 | R644Q | 1 | 0.13 |
| STAB2 | 666 | D666G | 1 | 0.13 |
| STAB2 | 787 | R787W | 1 | 0.13 |
| STAB2 | 845 | Y845H | 1 | 0.13 |
| STAB2 | 941 | K941R | 1 | 0.13 |
| STAB2 | 961 | T961I | 1 | 0.13 |
| STAB2 | 1035 | S1035P | 1 | 0.13 |
| STAB2 | 1047 | K1047I | 1 | 0.13 |
| STAB2 | 1102 | T1102I | 1 | 0.13 |
| STAB2 | 1118 | G1118V | 1 | 0.13 |
| STAB2 | 1126 | V1126L | 1 | 0.13 |
| STAB2 | 1132 | R1132H | 1 | 0.13 |
| STAB2 | 1200 | R1200W | 1 | 0.13 |
| STAB2 | 1259 | H1259Y | 1 | 0.13 |
| STAB2 | 1274 | N1274D | 1 | 0.13 |
| STAB2 | 1380 | G1380D | 1 | 0.13 |
| STAB2 | 1408 | Q1408K | 1 | 0.13 |
| STAB2 | 1460 | A1460V | 1 | 0.13 |
| STAB2 | 1486 | A1486V | 1 | 0.13 |
| STAB2 | 1541 | N1541K | 1 | 0.13 |
| STAB2 | 1570 | A1570L | 1 | 0.13 |
| STAB2 | 1597 | G1597S | 1 | 0.13 |
| STAB2 | 1603 | L1603F | 1 | 0.13 |
| STAB2 | 1639 | A1639V | 1 | 0.13 |
| STAB2 | 1745 | T1745K | 1 | 0.13 |
| STAB2 | 1782 | P1782H | 1 | 0.13 |
| STAB2 | 1787 | L1787F | 1 | 0.13 |
| STAB2 | 1863 | V1863L | 1 | 0.13 |
| STAB2 | 1889 | R1889H | 1 | 0.13 |
| STAB2 | 1893 | F1893L | 2 | 0.27 |
| STAB2 | 1898 | A1898D | 1 | 0.13 |
| STAB2 | 1919 | G1919A | 1 | 0.13 |
| STAB2 | 1996 | A1996V | 2 | 0.27 |
| STAB2 | 2139 | A2139T | 1 | 0.13 |
| STAB2 | 2210 | K2210R | 1 | 0.13 |
| STAB2 | 2282 | S2282N | 1 | 0.13 |
| STAB2 | 2342 | R2342H | 2 | 0.27 |
| STAB2 | 2355 | G2355S | 1 | 0.13 |
| STAB2 | 2374 | R2374Q | 1 | 0.13 |
| STAB2 | 2377 | E2377K | 1 | 0.13 |
| STAB2 | 2450 | P2450S | 1 | 0.13 |
| STAB2 | 2456 | T2456N | 1 | 0.13 |
| STAB2 | 2519 | Y2519S | 3 | 0.40 |
| STAB2 | 2540 | R2540W | 1 | 0.13 |
| STAB2 | 2542 | L2542P | 1 | 0.13 |

“LOC728763” or “CROCC2” as used herein refers to a gene of ciliary rootlet coiled-coil, rootletin family member 2, or the protein encoded by the gene. Exemplary mutations in LOC728763 useful in the embodiments disclosed herein are provided in

Tables 1.9 below. All mutations therein were reported based on genetic analysis of patients' tumor samples in Example 3.

TABLE 1.9
Exemplary LOC728763 Mutations

| Mutation | # |
|---|---|
| A169S | 1 |
| A271fs | 1 |
| A276V | 1 |
| C322R | 1 |
| D353G | 1 |
| D388V | 1 |
| E266K | 47 |
| G307E | 1 |
| L170R | 1 |
| L313F | 3 |
| Q325R | 1 |
| R148H | 1 |
| R251W | 1 |
| R282H | 45 |
| R291C | 1 |
| R374P | 3 |
| R56W | 1 |
| R58Q | 1 |
| V180A | 1 |

“SPATA31C2”, as used herein, refers to SPATA31 super family C member 2 gene, or the protein encoded by the gene. Exemplary mutations in SPATA31C2 useful in the embodiments disclosed herein are provided in Tables 1.10 below. All mutations therein were reported based on genetic analysis of patients' tumor samples in Example 3.

TABLE 1.10
Exemplary SPATA31C2 Mutations

| Gene | Position | mutation | # | Percent |
|---|---|---|---|---|
| SPATA31C2 | 64 | R64C | 1 | 0.13 |
| SPATA31C2 | 65 | His65_Arg70del | 2 | 0.27 |
| SPATA31C2 | 70 | R70_P71ins12 | 36 | 4.81 |
| SPATA31C2 | 74 | R74Q, R74W | 2 | 0.27 |
| SPATA31C2 | 135 | R135fs | 1 | 0.13 |
| SPATA31C2 | 148 | T148R | 55 | 7.34 |
| SPATA31C2 | 160 | S160C | 1 | 0.13 |
| SPATA31C2 | 310 | R310H | 15 | 2.00 |
| SPATA31C2 | 798 | R798S | 1 | 0.13 |
| SPATA31C2 | 807 | P807A | 1 | 0.13 |
| SPATA31C2 | 1037 | K1037N | 1 | 0.13 |
| SPATA31C2 | 1056 | M1056L | 57 | 7.61 |
| SPATA31C2 | 1118 | T1118A | 1 | 0.13 |
| SPATA31C2 | 1124 | R1124H | 3 | 0.40 |
| SPATA31C2 | 1135 | X1135Q | 1 | 0.13 |

“PTEN” as used herein, refers to phosphatase and tensin homolog gene, or the protein encoded by the gene. Exemplary mutations in PTEN useful in the embodiments disclosed herein are provided in Tables 1.11 below. All mutations therein were reported based on genetic analysis of patients' tumor samples in Example 3.

TABLE 1.11
Exemplary in PTEN Mutations

| Gene | Position | Mutation | # | Percent |
|---|---|---|---|---|
| PTEN | 1 | Met1_Ala3del | 1 | 0.13 |
| PTEN | 17 | Q17* | 1 | 0.13 |
| PTEN | 24 | D24fs | 1 | 0.13 |
| PTEN | 28 | I28N | 1 | 0.13 |
| PTEN | 33 | I33T | 1 | 0.13 |
| PTEN | 35 | M35R | 1 | 0.13 |
| PTEN | 68 | Y68D | 1 | 0.13 |
| PTEN | 73 | Glu73_His75delinsAsp | 1 | 0.13 |
| PTEN | 86 | A86P | 1 | 0.13 |
| PTEN | 92 | D92fs | 1 | 0.13 |
| PTEN | 97 | Q97fs | 1 | 0.13 |
| PTEN | 99 | E99fs | 1 | 0.13 |
| PTEN | 103 | Pro103_Leu112dup | 1 | 0.13 |
| PTEN | 107 | D107Y | 1 | 0.13 |
| PTEN | 114 | E114* | 2 | 0.27 |
| PTEN | 118 | H118fs | 1 | 0.13 |
| PTEN | 123 | H123Q | 1 | 0.13 |
| PTEN | 126 | A126D | 1 | 0.13 |
| PTEN | 128 | K128N, K128fs | 3 | 0.40 |
| PTEN | 129 | G129E | 2 | 0.27 |
| PTEN | 130 | R130*, R130fs | 2 | 0.27 |
| PTEN | 132 | G132D, G132V | 2 | 0.27 |
| PTEN | 134 | M134T | 1 | 0.13 |
| PTEN | 135 | I135M, I135fs | 2 | 0.27 |
| PTEN | 136 | C136Y, C136W | 2 | 0.27 |
| PTEN | 138 | Y138C | 1 | 0.13 |
| PTEN | 139 | L139fs | 1 | 0.13 |
| PTEN | 144 | K144fs | 1 | 0.13 |
| PTEN | 146 | L146fs | 2 | 0.27 |
| PTEN | 157 | E157fs | 1 | 0.13 |
| PTEN | 170 | S170fs | 1 | 0.13 |
| PTEN | 173 | R173G | 1 | 0.13 |
| PTEN | 174 | Y174H | 1 | 0.13 |
| PTEN | 182 | L182fs | 1 | 0.13 |
| PTEN | 183 | K183fs | 1 | 0.13 |
| PTEN | 184 | N184fs | 1 | 0.13 |
| PTEN | 210 | T210fs | 1 | 0.13 |
| PTEN | 211 | C211*, C211Y | 2 | 0.27 |
| PTEN | 233 | R233* | 1 | 0.13 |
| PTEN | 235 | E235fs | 1 | 0.13 |
| PTEN | 240 | Y240fs | 1 | 0.13 |
| PTEN | 241 | F241C, F241fs | 2 | 0.27 |
| PTEN | 246 | P246fs | 1 | 0.13 |
| PTEN | 249 | V249E | 1 | 0.13 |
| PTEN | 258 | F258fs | 1 | 0.13 |
| PTEN | 260 | K260* | 1 | 0.13 |
| PTEN | 289 | K289* | 1 | 0.13 |
| PTEN | 293 | G293fs | 1 | 0.13 |
| PTEN | 296 | C296* | 1 | 0.13 |
| PTEN | 299 | E299* | 1 | 0.13 |
| PTEN | 305 | S305fs | 1 | 0.13 |
| PTEN | 314 | E314* | 1 | 0.13 |
| PTEN | 319 | T319fs | 1 | 0.13 |
| PTEN | 329 | N329fs | 2 | 0.27 |
| PTEN | 336 | Y336* | 1 | 0.13 |

“ARVCF” as used herein, refers to Armadillo Repeat protein deleted in Velo-Cardio-Facial syndrome (ARVCF), or the protein encoded by the gene.

The exemplary mutations described in above Tables 1.1 to 1.11, are actual mutation data obtained and reported from Example 3. The respective transcript isoforms of the reported mutations are described in below Table 1.12. Sequences associated with the transcript accession numbers in Table 1.12 are available, for example at the National Center for Biotechnology Information (NCBI). As used in Tables 1.1 to 1.11, “#” refers to the number of patients in which such mutation was detected, “percentage” refers to the percentage of patients in the whole group that such mutation was detected.

TABLE 1.12
Respective Transcript Isoforms of the Mutations Reported

| Gene | Transcript Accession No. |
|---|---|
| ABCA1 | NM_005502.3 |
| CD163L1 | NM_174941.4 |
| DNMT1 | NM_001130823.1 |
| FOXO1 | NM_002015.3 |
| IL16 | NM_001172128.1 (Table 1.6A) |
| IL16 | XM_005254343.1 (Table 1.6B) |
| LOC728763 | XM_003118518.2 |
| MC1R | NM_002386.3 |
| MYH7B | NM_020884.3 |
| SPATA31C2 | NM_001166137.1 |
| STAB2 | NM_017564.9 |
| PTEN | NM_000314.4 |
| ARVCF | NM_001670.3 |

It is appreciated that for each of the mutations described in the above Tables 1.1 to 1.11, the same mutation in the DNA of the respective gene may be expressed through a different transcript isoform and thus results in a mutation of the amino acid with a different position number in the respective protein. Thus, when referring to “protein altering mutation” of a specific gene, according to one of Tables 1.1 to 1.11, counterpart mutations expressed through other transcript isoforms of the same gene are also contemplated.

“Patient” or “subject” refers to any single subject for which therapy is desired or that is participating in a clinical trial, epidemiological study or used as a control, including humans and mammalian veterinary patients such as cattle, horses, dogs, and cats.

“PD-1 axis binding antagonist” refers to a molecule that inhibits the interaction of a PD-1 axis binding partner with either one or more of its binding partner, so as to remove T-cell dysfunction resulting from signaling on the PD-1 signaling axis, with a result being to restore or enhance T-cell function. As used herein, a PD-1 axis binding antagonist includes a PD-1 antagonist, a PD-L1 antagonist and a PD-L2 antagonist.

“PD-L1 antagonist” means any chemical compound or biological molecule that blocks binding of PD-L1 expressed on a cancer cell to PD-1. In any of the treatment method, medicaments and uses of the present invention in which a human subject is being treated, the PD-L1 antagonist blocks binding of human PD-L1 to human PD-1.

PD-L1 antagonists useful in the any of the treatment methods, medicaments, and uses of the present invention include a monoclonal antibody (mAb) which specifically binds to PD-L1, and preferably specifically binds to human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments. As used herein, an anti-human PD-L1 antibody refers to an antibody that specifically binds to mature human PD-L1. A mature human PD-L1 molecule consists of amino acids 19-290 of the following sequence (SEQ ID NO: 16):

(SEQ ID NO: 16)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET.

Examples of PD-1 axis binding antagonist and useful in the treatment method, medicaments and uses of the present invention, are described in WO2013079174, WO2015061668, WO2010089411, WO/2007/005874, WO/2010/036959, WO/2014/100079, WO2013/019906, WO/2010/077634, and U.S. Pat. Nos. 8,552,154, 8,779,108, and 8,383,796. Specific PD-1 axis binding antagonist useful in the treatment method, medicaments and uses of the present invention include, for example without limitation: pembrolizumab (aka MK-3475, an anti-PD-1 IgG4 monoclonal antibody) nivolumab (aka BMS-936558 or MDX1106, an anti-PD-1 IgG4 monoclonal antibody), cemiplimab (aka REGN-2810, an anti-PD-1 antibody), avelumab (aka MSB0010718C, an anti-PD-L1 IgG1 monoclonal antibody), atezolizumab (aka MPDL3280A an IgG1-engineered, anti-PD-L1 antibody), BMS-936559 (a fully human, anti-PD-L1, IgG4 monoclonal antibody), MED14736 (aka durvalumab, an engineered IgG1 kappa anti-PD-L1 monoclonal antibody with triple mutations in the Fc domain to remove antibody-dependent, cell-mediated cytotoxic activity). Additional exemplary PD-1 axis binding antagonist useful in the treatment method, medicaments and uses of the present invention include SHR1210 (anti-PD-1 antibody), KN035 (anti-PD-L1 antibody), 161308 (anti-PD-1 antibody), PDR001 (anti-PD-1 antibody), BGB-A317 (anti-PD-1 antibody), BCD-100 (anti-PD-1 antibody), JS001 (anti-PD-1 antibody), as described in Darvin et al. Experimental & Molecular Medicine (2018) 50:165, the disclosure of which is herein incorporated by reference in its entirety. In some embodiments, a PD-1 axis binding antagonist is a small molecule PD-1 or PD-L1 antagonist (e.g. CA-170), as described in Yang et al Med. Res. Rev. (2019), 39, pp 265-301, the disclosure of which is herein incorporated by reference in its entirety.

Other PD-1 axis binding antagonist useful in the any of the treatment method, medicaments and uses of the present invention also include an immunoadhesin that specifically binds to PD-1 or PD-L1, and preferably specifically binds to human PD-1 or PD-L1, e.g., a fusion protein containing a portion that binds to PD-1 or PD-L1, fused to a constant region such as an Fc region of an immunoglobulin molecule.

Table 2 below provides sequences of some of the exemplary antibodies that are PD-1 axis binding antagonist for use in the treatment method, medicaments and uses of the present invention. CDRs are underlined for mAb7 and mAb15. The mAB7 is also known as RN888 or PF-6801591. mAb7 (aka RN888) and mAb15 are disclosed in International Patent Publication No. WO2016/092419, the disclosure of which is hereby incorporated by reference in its entirety.

TABLE 2
Sequences of Exemplary PD-1 Axis Binding
Antagonist

| | |
|---|---|
| mAb7(aka RN888) | QVQLVQSGAEVKKPGASVKVSCKASGYTFT |
| or mAb15 full- | SYWINWVRQAPGQGLEWMGNIYPGSSLTNY |
| length heavy | NEKFKNRVTMTRDTSTSTVYMELSSLRSED |
| chain | TAVYYCARLSTGTFAYWGQGTLVTVSSAST |
| | KGPSVFPLAPCSRSTSESTAALGCLVKDYF |
| | PEPVTVSWNSGALTSGVHTFPAVLQSSGLY |
| | SLSSVVTVPSSSLGTKTYTCNVDHKPSNTK |
| | VDKRVESKYGPPCPPCPAPEFLGGPSVFLF |
| | PPKPKDTLMISRTPEVTCVVVDVSQEDPEV |
| | QFNWYVDGVEVHNAKTKPREEQFNSTYRVV |
| | SVLTVLHQDWLNGKEYKCKVSNKGLPSSIE |
| | KTISKAKGQPREPQVYTLPPSQEEMTKNQV |
| | SLTCLVKGFYPSDIAVEWESNGQPENNYKT |
| | TPPVLDSDGSFFLYSRLTVDKSRWQEGNVF |
| | SCSVMHEALHNHYTQKSLSLSLGK |
| | (SEQ ID NO: 1) |
| mAb7 or | QVQLVQSGAEVKKPGASVKVSCKASGYTFT |
| mAb15 full- | SYWINWVRQAPGQGLEWMGNIYPGSSLTNY |
| length heavy | NEKFKNRVTMTRDTSTSTVYMELSSLRSED |
| chain without | TAVYYCARLSTGTFAYWGQGTLVTVSSAST |
| the C-terminal | KGPSVFPLAPCSRSTSESTAALGCLVKDYF |
| lysine | PEPVTVSWNSGALTSGVHTFPAVLQSSGLY |
| | SLSSVVTVPSSSLGTKTYTCNVDHKPSNTK |
| | VDKRVESKYGPPCPPCPAPEFLGGPSVFLF |
| | PPKPKDTLMISRTPEVTCVVVDVSQEDPEV |
| | QFNWYVDGVEVHNAKTKPREEQFNSTYRVV |
| | SVLTVLHQDWLNGKEYKCKVSNKGLPSSIE |
| | KTISKAKGQPREPQVYTLPPSQEEMTKNQV |
| | SLTCLVKGFYPSDIAVEWESNGQPENNYKT |
| | TPPVLDSDGSFFLYSRLTVDKSRWQEGNVF |
| | SCSVMHEALHNHYTQKSLSLSLG |
| | (SEQ ID NO: 2) |
| mAb7 full- | DIVMTQSPDSLAVSLGERATINCKSSQSLW |
| length light | DSGNQKNFLTWYQQKPGQPPKLLIYWTSYR |
| chain | ESGVPDRFSGSGSGTDFTLTISSLQAEDVA |
| | VYYCQNDYFYPHTFGGGTKVEIKRGTVAAP |
| | SVFIFPPSDEQLKSGTASVVCLLNNFYPRE AKVQWKVDNALQSGNSQESVTEQDSKDSTY |
| | SLSSTLTLSKADYEKHKVYACEVTHQGLSS |
| | PVTKSFNRGEC (SEQ ID NO: 3) |
| mAb7 light | DIVMTQSPDSLAVSLGERATINCKSSQSLW |
| chain | DSGNQKNFLTWYQQKPGQPPKLLIYWTSYR |
| variable | ESGVPDRFSGSGSGTDFTLTISSLQAEDVA |
| region | VYYCQNDYFYPHTFGGGTKVEIK |
| | (SEQ ID NO: 4) |
| mAB7 and mAB15 | QVQLVQSGAEVKKPGASVKVSCKASGYTFT |
| heavy chain | SYWINWVRQAPGQGLEWMGNIWPGSSLTNY |
| variable | NEKFKNRVTMTRDTSTSTVYMELSSLRSED |
| region | TAVYYCARLLTGTFAYWGQGTLVTVSS |
| | (SEQ ID NO: 5) |
| mAb15 light | DIVMTQSPDSLAVSLGERATINCKSSQSLW |
| chain | DSGNQKNFLTWYQQKPGQPPKLLIYWTSYR |
| variable | ESGVPDRFSGSGSGTDFTLTISSLQAEDVA |
| region | VYYCQNDYFYPHTFGGGTKVEIK |
| | (SEQ ID NO: 6) |
| Nivolumab, | QVQLVESGGGWQPGRSLRLDCKASGITFSN |
| MDX1106, full | SGMHWVRQAPGKGLEWVAVrWYDGSKRYYA |
| length heavy | DSVKGRFTISRDNSKNTLFLQMNSLRAEDT |
| chain | AVYYCATNDDYWGQGTLVTVSSASTKGPSV |
| From | FPLAPCSRSTSESTAALGCLVDYFPEPVTV |
| WO2006/121168 | SWNSGALTSGVHTFPAVLQSSGLYSLSSVV |
| | TVPSSSLGTTYTCNVDHKPSNTKVDRVESY |
| | GPPCPPCPAPEFLGGPSVFLFPPKPKDTLM |
| | ISRTPEVTCWVDVSQEDPEVQFNWYYDGVE |
| | VHNATKPREEQFNSTYRVVSVLTVLHQDWL |
| | NGKEYKCKVSNKGLPSSIEKTISKA |
| | GQPREPQVYTLPPSQEEMTKNQVSLTCLVK |
| | GFYPSDIAVEWESNGQPEKNYKTTPPVLDS |
| | DGSFFLYSRLTVDKSRWQEGNVFSCSVMHE |
| | ALHNHYTQKSLSLSLGK |
| | (SEQ ID NO: 7) |
| Nivolumab, | EIVLTQSPATLSLSPGERATLSCRASQSVS |
| MDX1106, full | SYLAWYQQPGQAPRLLIYDASNRATGIPAR |
| length light | FSGSGSGTDFTLTISSLEPEDFAVYYCQQS |
| chain | SNWPRTFGQGTKVEIRTVAAPSVFIFPPSD |
| From | EQLSGTASVVCLLNNFYPREAVQWKVDNAL |
| WO2006/121168 | QSGNSQESVTEQDSDSTYSLSSTLTLSKAD |
| | YEKHKVYACEVTHQGLSSPVT SFNRGEC |
| | (SEQ ID NO: 8) |
| Pembrolizumab, | QVQLVQSGVEVKKPGASVKVSCKASGYTFT |
| MK3475, full | NYYMYWVRQAPGQGLEWMGGINPSNGGTNF |
| length heavy | NEKFKNRVTLTTDSSTTTAYMELKSLQFDD |
| chain | TAVYYCARRDYRFDMGFDYWGQGTTVTVSS |
| From | ASTKGPSVFPLAPCSRSTSESTAALGCLVK |
| WO2009114335 | DYFPEPVTVSWNSGALTSGVHTFPAVLQSS |
| | GLYSLSSVVTVPSSSLGTKTYTCNVDHKPS |
| | NTKVDKRVESKYGPPCPPCPAPEFLGGPSV |
| | FLFPPKPKDTLMISRTPEVTCVVVDVSQED |
| | PEVQFNWYVDGVEVHNAKTKPREEQFNSTY |
| | RVVSVLTVLHQDWLNGKEYKCKVSNKGLPS |
| | SIEKTISKAKGQPREPQVYTLPPSQEEMTK |
| | NQVSLTCLVKGFYPSDIAVEWESNGQPENN |
| | YKTTPPVLDSDGSFFLYSRLTVDKSRWQEG |
| | NVFSCSVMHEALHNHYTQKSLSLSLGK |
| | (SEQ ID NO: 9) |
| Pembrolizumab, | EIVLTQSPATLSLSPGERATLSCRASKGVS |
| MK3475, full | TSGYSYLHWYQQKPGQAPRLLIYLASYLES |
| length light | GVPARFSGSGSGTDFTLTISSLEPEDFAVY |
| chain | YCQHSRDLPLTFGGGTKVEIKRTVAAPSVF |
| From | IFPPSDEQLKSGTASVVCLLNNFYPREAKV |
| WO2009114335 | QWKVDNALQSGNSQESVTEQDSKDSTYSLS |
| | STLTLSKADYEKHKVYACEVTHQGLSSPVT |
| | KSFNRGEC (SEQ ID NO: 10) |
| AMP224, | LFTVTVPKELYIIEHGSNVTLECNFDTGSH |
| without signal | VNLGAITASLQKVENDTSPHRERATLLEEQ |
| sequence | LPLGKASFHIPQVQVRDEGQYQCIIIYGVA |
| From | WDYKYLTLKVKASYRKINTHILKVPETDEV |
| WO2010027827 | ELTCQATGYPLAEVSWPNVSVPANTSHSRT |
| and | PEGLYQVTSVLRLKPPPGRNFSCVFWNTHV |
| WO2011066342 | RELTLASIDLQSQMEPRTHPTWEPKSCDKT |
| | HTCPPCPAPELLGGPSVFLFPPKPKDTLMI |
| | SRTPEVTCWVDVSHEDPEVKFNWYVDGVEV |
| | HNAKTKPREEQYNSTYRWSVLTVLHQDWLN |
| | GKEYKCKVSNKALPAPIEKTISKAKGQPRE |
| | PQVYTLPPSRDELTKNQV SLTCLVKGFY PSDIAVEWES NGQPENNYKT TPPVLDSD |
| | GSFFLYSKLTVDKSRWQQGNVFSCSVMHEA |
| | LHNHYTQKSLSLSPGK |
| | (SEQ ID NO: 11) |
| YW243.55.S70 | EVQLVESGGGLVQPGGSLRLSCAASGFTFS |
| heavy chain | DSWIHWVRQAPGKGLEWVAWISPYGGSTYY |
| From | ADSVKGRFTISADTSKNTAYLQMNSLRAED |
| WO2010077634 | TAVYYCARRHWPGGFDYWGQGTLVTVSA |
| | (SEQ ID NO: 12) |
| YW243.55.570 | DIQMTQSPSSLSASVGDRVTITCRASQDVS |
| light chain | TAVAWYQQKPGKAPKLLIYSASFLYSGVPS |
| From | RFSGSGSGTDFTLTISSLQPEDFATYYCQQ |
| WO2010077634 | YLYH PATFGQGTKVEIKR (SEQ ID |
| | NO: 13) |
| avelumab | EVQLLESGGGLVQPGGSLRLSCAASGFTFS |
| heavy chain | SYIMWVRQAPGKGLEWVSSIYPSGGITFYA |
| variable | DKGRFTISRDNSKNTLYLQMNSLRAEDTAV |
| region From | YYCARIKLGTVTTVDYWGQ GTLVTVSS |
| WO13079174 | (SEQ ID NO: 14) |
| avelumab | QSALTQPASVSGSPGQSITISCTGTSSDVG |
| light chain | GYNYVSWYQQHPGKAPKLMIYDVSNRPSGV |
| variable | SNRFSGSKSGNTASLTISGLQAEDEADYYC |
| region From | SSYTSSSTRVFGTGTKVTVL (SEQ ID |
| WO13079174 | NO: 15) |

Table 3 below provides the sequences of the anti-PD-L1 antibody avelumab for use in the treatment methods, medicaments and uses of the present invention. Avelumab is disclosed as A09-246-2, in International Patent Publication No. WO2013/079174, the disclosure of which is hereby incorporated by reference in its entirety.

TABLE 3
ANTI-HUMAN PD-L1 MONOCLONAL ANTIBODY AVELUMAB
SEQUENCES

| | |
|---|---|
| Heavy chain | SYIMM (SEQ ID NO: 17) |
| CDR1 (CDRH1) | |
| Heavy chain | SIYPSGGITFY (SEQ ID NO: 18) |
| CDR2 (CDRH2) | |
| Heavy chain | IKLGTVTTVDY (SEQ ID NO: 19) |
| CDR3 (CDRH3) | |
| Light chain | TGTSSDVGGYNYVS (SEQ ID NO: 20) |
| CDR1 (CDRL1) | |
| Light chain | DVSNRPS (SEQ ID NO: 21) |
| CDR2 (CDRL2) | |
| Light chain | SSYTSSSTRV (SEQ ID NO: 22) |
| CDR3 (CDRL3) | |
| Heavy chain | EVQLLESGGGLVQPGGSLRLSCAASGFTFSSYIMMWVR |
| variable | QAPGKGLEWVSSIYPSGGITFYADKGRFTISRDNSKNT |
| region (VR) | LYLQMNSLRAEDTAVYYCARIKLGTVTTVDYWGQGTLV |
| | TVSS (SEQ ID NO: 14) |
| Light | QSALTQPASVSGSPGQSITISCTGTSSDVGGYNYVSWY |
| chain VR | QQHPGKAPKLMIYDVSNRPSGVSNRFSGSKSGNTASLT |
| | ISGLQAEDEADYYCSSYTSSSTRVFGTGTKVTVL |
| | (SEQ ID NO: 15) |
| Heavy chain | EVQLLESGGGLVQPGGSLRLSCAASGFTFSSYIMMWVR |
| | QAPGKGLEWVSSIYPSGGITFYADTVKGRFTISRDNSK |
| | NTLYLQMNSLRAEDTAVYYCARIKLGTVTTVDYWGQGT |
| | LVTVSSASTKGPSVFPLAPSSKSTSGGTAALGCLVKDY |
| | FPEPVTVSWNSGALTSGVHTFPAVLQSSGLYSLSSVVT |
| | VPSSSLGTQTYICNVNHKPSNTKVDKKVEPKSCDKTHT |
| | CPPCPAPELLGGPSVFLFPPKPKDTLMISRTPEVTCVV |
| | VDVSHEDPEVKFNWYVDGVEVHNAKTKPREEQYNSTYR |
| | VVSVLTVLHQDWLNGKEYKCKVSNKALPAPIEKTISKA |
| | KGQPREPQVYTLPPSRDELTKNQVSLTCLVKGFYPSDI |
| | AVEWESNGQPENNYKTTPPVLDSDGSFFLYSKLTVDKS |
| | RWQQGNVFSCSVMHEALHNHYTQKSLSLSPGK |
| | (SEQ ID NO: 23) |
| Light | QSALTQPASVSGSPGQSITISCTGTSSDVGGYNYVSWY |
| chain | QQHPGKAPKLMIYDVSNRPSGVSNRFSGSKSGNTASLT |
| | ISGLQAEDEADYYCSSYTSSSTRVFGTGTKVTVLGQPK |
| | ANPTVTLFPPSSEELQANKATLVCLISDFYPGAVTVAW |
| | KADGSPVKAGVETTKPSKQSNNKYAASSYLSLTPEQWK |
| | SHRSYSCQVTHEGSTVEKTVAPTECS |
| | (SEQ ID NO: 24) |

“PD-L1” expression as used herein means any detectable level of expression of PD-L1 protein on the cell surface or of PD-L1 mRNA within a cell or tissue. PD-L1 protein expression may be detected with a diagnostic PD-L1 antibody in an IHC assay of a tumor tissue section or by flow cytometry. Alternatively, PD-L1 protein expression by tumor cells may be detected by PET imaging, using a binding agent (e.g., antibody fragment, affibody and the like) that specifically binds to PD-L1. Techniques for detecting and measuring PD-L1 mRNA expression include RT-PCR and real-time quantitative RT-PCR.

Several approaches have been described for quantifying PD-L1 protein expression in IHC assays of tumor tissue sections. See, e.g., Thompson, R. H., et al., PNAS 101 (49); 17174-17179 (2004); Thompson, R. H. et al., Cancer Res. 66:3381-3385 (2006); Gadiot, J., et al., Cancer 117:2192-2201 (2011); Taube, J. M. et al., Sci Transl Med 4, 127ra37 (2012); and Toplian, S. L. et al., New Eng. J Med. 366 (26): 2443-2454 (2012).

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. For tumor cells, PD-L1 expression is counted as negative if the score is <5% score and positive if the score is 5%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is ≥5.

The level of PD-L1 mRNA expression may be compared to the mRNA expression levels of one or more reference genes that are frequently used in quantitative RT-PCR, such as ubiquitin C.

In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/ or mRNA) by an appropriate control. For example, a control PD-L1 protein or mRNA expression level may be the level quantified in nonmalignant cells of the same type or in a section from a matched normal tissue.

“RECIST 1.1 Response Criteria” as used herein means the definitions set forth in Eisenhauer et al., E. A. et al., Eur. J Cancer 45:228-247 (2009) for target lesions or nontarget lesions, as appropriate based on the context in which response is being measured.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

An “effective response” of a patient or a patient's “responsiveness” to treatment with a medicament and similar wording refers to the clinical or therapeutic benefit imparted to a patient at risk for, or having a, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

“Tissue Section” refers to a single part or piece of a tissue sample, e.g., a thin slice of tissue cut from a sample of a normal tissue or of a tumor.

“Treat” or “treating” a cancer as used herein means to administer a combination therapy of a PD-1 axis binding antagonist and another therapeutic agent to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009)). For example, with respect to tumor growth inhibition, according to National Cancer Institute (NCI) standards, a T/C less than or equal to 42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination of the invention is any of partial response (PR), complete response (CR), overall response (OR), progression free survival (PFS), disease free survival (DFS) and overall survival (OS). PFS, also referred to as “Time to Tumor Progression” indicates the length of time during and after treatment that the cancer does not grow, and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naive or untreated subjects or patients. In some embodiments, response to a combination of the invention is any of PR, CR, PFS, DFS, OR, or OS that is assessed using Response Evaluation Criteria in Solid Tumors (RECIST) 1.1 response criteria. The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

The terms “treatment regimen”, “dosing protocol” and dosing regimen are used interchangeably to refer to the dose and timing of administration of each therapeutic agent in a combination of the invention.

As used herein, “treatment” is an approach for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of tumor, remission of a PD-1 axis associated disease (e.g., cancer), decreasing symptoms resulting from a PD-1 axis associated disease (e.g., cancer), increasing the quality of life of those suffering from a PD-1 axis associated disease (e.g., cancer), decreasing the dose of other medications required to treat a PD-1 axis associated disease (e.g., cancer), delaying the progression of a PD-1 axis associated disease (e.g., cancer), curing a PD-1 axis associated disease (e.g., cancer), and/or prolong survival of patients having a PD-1 axis associated disease (e.g., cancer).

“Ameliorating” means a lessening or improvement of one or more symptoms as compared to not administering a therapy or medicament. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various PD-1 axis associated diseases or conditions (such as for example advanced RCC), decreasing the dose of other medications required to treat the disease, enhancing the effect of another medication, and/or delaying the progression of the PD-1 axis associated disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved. In reference to the treatment of cancer, an effective amount refers to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression

The terms “for improving progression free survival” in the context of the present invention refer, with respect to a patient within a patient group, to the average length of time during and after treatment in which a patient's disease does not get worse. As the skilled person will appreciate, a patient's progression free survival is improved or enhanced, if the patient belongs to a subgroup of patients that has a significantly longer mean length of time during which the disease does not get worse compared to another subgroup of patients.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumors, throughout the body, including lymph nodes and bone narrow. Tumor burden can be determined by a variety of methods known in the art, such as, e.g. by measuring the dimensions of tumors upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumors upon removal from the subject, e.g., using calipers, or while in the body using imaging techniques, e.g., bone scan, ultrasound, CT or MRI scans.

“Variable regions” or “V region” as used herein means the segment of IgG chains which is variable in sequence between different antibodies. It extends to Kabat residue 109 in the light chain and 113 in the heavy chain.

“VEGF pathway inhibitor” as used herein means a molecule that is an inhibitor of vascular endothelial growth factor (VEGF)or vascular endothelial growth factor receptor VEGFR.

“VEGF inhibitor” means a molecule that is an inhibitor of vascular endothelial growth factor (VEGF). In an embodiment, a “VEGF inhibitor” means a small molecule inhibitor of VEGF. In another embodiment, a “VEGF inhibitor” means an anti VEGF antibody. In another embodiment, a “VEGF inhibitor” means a VEGF trap. Specific VEGF inhibitors useful as the VEGF inhibitor in the treatment method, medicaments and uses of the present invention, include bevacizumab, HuMV833, pegaptanib aptamer, ranibizumab and Aflibercept.

“VEGFR inhibitor” means a molecule that is an inhibitor of VEGFR. In an embodiment, a “VEGFR inhibitor” means a small molecule inhibitor of VEGFR. In another embodiment, a “VEGFR inhibitor” means an anti VEGFR antibody. In another embodiment, a “VEGFR inhibitor” means a VEGFR trap. Specific VEGFR inhibitors useful as the VEGFR inhibitor in the treatment method, medicaments and uses of the present invention, include axitinib, sunitinib, sorafenib, tivozanib, and bevacizumab.

In an embodiment of the treatment method, medicaments and uses of the present invention, the VEGFR inhibitor is the compound, N-methyl-2-[3-((E)-2-pyridin-2-yl-vinyl)-1H-indazol-6-ylsulfanyl]-benzamide or 6-[2-(methylcarbamoyl)phenylsulfanyl]-3-E-[2-(pyridin-2-yl)ethenyl]indazole, of the following structure:

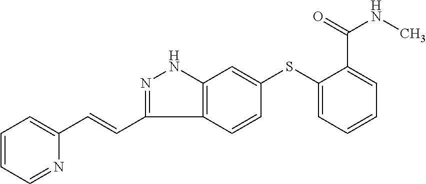 [SMILES: CNC(=O)c1ccccc1Sc1ccc2c(/C=C/c3ccccn3)n[nH]c2c1]

which is known as axitinib or AG-013736.

Axitinib, as well as pharmaceutically acceptable salts thereof, is described in U.S. Pat. No. 6,534,524. Methods of making axitinib are described in U.S. Pat. Nos. 6,884,890 and 7,232,910, in U.S. Publication Nos. 2006-0091067 and 2007-0203196 and in International Publication No. WO 2006/048745. Dosage forms of axitinib are described in U.S. Publication No. 2004-0224988. Polymorphic forms and pharmaceutical compositions of axitinib are also described in U.S. Publication Nos. 2006-0094763, 2008-0274192 and 2010-0179329 and International Publication No. WO 2013/046133. The patents and patent applications listed above are incorporated herein by reference.

Axitinib is understood to include reference to salts thereof, unless otherwise indicated. Axitinib is basic in nature and capable of forming a wide variety of salts with various inorganic and organic acids. The term “salts”, as employed herein, denotes acidic salts formed with inorganic and/or organic acids. Pharmaceutically acceptable salts of axitinib may be formed, for example, by reacting axitinib with an amount of acid, such as an equivalent amount, in a medium such as one in which the salt precipitates or in an aqueous medium followed by lyophilization.

Exemplary acid addition salts of the compound of Formula I, and other VEGF pathway inhibitors, as applicable, include acetates, ascorbates, benzoates, benzenesulfonates, bisulfates, borates, butyrates, citrates, camphorates, cam phorsulfonates, fumarates, hydrochlorides, hydrobrom ides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

All such acid salts are intended to be pharmaceutically acceptable salts within the scope of axitinib, as used in the present invention and all acid salts are considered equivalent to the free forms of the corresponding compound for purposes of the invention.

Prodrugs of axitinib or other VEGF pathway inhibitors, as applicable, are also contemplated for use in the methods, medicaments and uses of the present invention. The term “prodrug”, as employed herein, denotes a compound that is a drug precursor which, upon administration to a subject, undergoes chemical conversion by metabolic or chemical processes to yield axitinib or a salt thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“DUX4 gene signature” refers to the expression level of one or more genes whose transcription is regulated by the protein DUX4. DUX4 protein is a transcription factor, and increased DUX4 protein expression may result in the increased expression of one or more genes whose transcription is regulated by DUX4. Exemplary genes that can be included in the DUX4 gene signature include ZSCAN4, PRAMEF1, SPRYDS, KHDC1L, MBD3L2, and TRIM43. In some embodiments, assessing the DUX4 gene signature may include assessing the expression of 1, 2, 3, 4, 5, or all 6 of ZSCAN4, PRAMEF1, SPRYDS, KHDC1L, MBD3L2, and TRIM43. Expression of the DUX4 gene may be assessed by measuring expression of the DUX4 gene or by measuring expression of the DUX4 gene signature. References herein to assessing the level “DUX4 expression” or the like include measuring either or both of the DUX4 gene or the DUX4 gene signature (unless the context clearly dictates that only the DUX4 gene or the only DUX4 gene signature is intended).

The genes UTS2, CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1, DUX4, ZSCAN4, PRAMEF1, SPRYDS, KHDC1L, MBD3L2, TRIM43, NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB, NOTCH2, ANK2, CAPN8, CBX4, CNTRL, CYP2W1, DMRTA1, EPHA2, GREB1, HBS1L, LAMA1, LOC728392, LYST, MYOM2, NOS3, PALM3, PLK5, PTPN13, RTL1, SCAP, SHROOM2, SLCO2B1, TBX2, TENM3, TNRC6A, TTC28, USP42, ZC3H3, EFCAB6, MAP3K6, and PTPDC1 are known to persons of skill in the art. Information about the genes is available at, for example, the National Center for Biotechnology Information (NCBI), the Universal Protein Resource (UniProt), The Human Protein Atlas, LifeMap Sciences (e.g. GeneCards), Catalogue Of Somatic Mutations In Cancer (COSMIC), and the National Cancer Institute (e.g. The Cancer Genome Atlas Program (TCGA)).

Exemplary sequence reference information for genes provided herein is listed below in Table 4. Sequences (e.g. mRNA and gene) associated with the transcript accession numbers in Table 4 are available, for example at the National Center for Biotechnology Information (NCBI). While reference information for a single isoform for each gene is provide in Table 4, additional isoforms are also contemplated and within the scope of the embodiments provided herein.

TABLE 4
Transcript accession numbers of genes provided herein

| | Transcript | | Transcript |
|---|---|---|---|
| Gene | Accession No. | Gene | Accession No. |
| ANK2 | NM_001148.6 | SLCO2B1 | NM_007256.5 |
| CAPN8 | NM_001143962.2 | TBX2 | NM_005994.4 |
| CBX4 | NM_003655.3 | TENM3 | NM_001080477.4 |
| CNTRL | NM_007018.5 | TNRC6A | NM_014494.4 |
| CYP2W1 | NM_017781.3 | TTC28 | NM_001145418.1 |
| DMRTA1 | NM_022160.3 | USP42 | NM_001365764.1 |
| EPHA2 | NM_001329090.2 | ZC3H3 | NM_015117.3 |
| GREB1 | NM_014668.4 | EFCAB6 | NM_022785.4 |
| HBS1L | NM_006620.4 | MAP3K6 | NM_004672.5 |
| LAMA1 | NM_005559.4 | PTPDC1 | NM_152422.4 |
| LOC728392 | NM_001162371.3 | UTS2 | NM_021995.2 |
| LYST | NM_000081.4 | CD3G | NM_000073.3 |
| MYOM2 | NM_003970.4 | CD3E | NM_000733.4 |
| NOS3 | NM_000603.5 | CD8B | NM_172213.4 |
| PALM3 | NM_001145028.2 | THEMIS | NM_001164685.1 |
| PLK5 | NM_001243079.2 | TRAT1 | NM_016388.4 |
| PTPN13 | NM_080683.3 | GRAP2 | NM_001291824.2 |
| RTL1 | NM_001134888.3 | CD247 | NM_198053.3 |
| SCAP | NM_012235.4 | CD2 | NM_001328609.2 |
| SHROOM2 | NM_001649.4 | CD96 | NM_198196.3 |
| PRF1 | NM_005041.5 | CD244 | NM_016382.4 |
| CD6 | NM_006725.5 | KLRD1 | NM_007334.2 |
| IL7R | NM_002185.5 | SH2D1A | NM_002351.5 |
| ITK | NM_005546.4 | CCL5 | NM_002985.3 |
| GPR18 | NM_005292.3 | XCL2 | NM_003175.4 |
| EOMES | NM_001278182.2 | CST7 | NM_003650.4 |
| SIT1 | NM_014450.3 | GFI1 | NM_005263.5 |
| NLRC3 | NM_178844.4 | KCNA3 | NM_002232.5 |
| PSTPIP1 | NM_003978.5 | DUX4 | NM_001306068.3 |
| ZSCAN4 | NM_152677.2 | KHDC1L | NM_001126063.3 |
| PRAMEF1 | NM_023013.4 | MBD3L2 | NM_144614.3 |
| SPRYD5 | NM_032681.4 | TRIM43 | NM_138800.3 |
| NRARP | NM_001004354.3 | TEK | NM_000459.5 |
| RAMP2 | NM_005854.3 | FLT1 | NM_002019.4 |
| ARHGEF15 | NM_173728.4 | GATA2 | NM_001145661.2 |
| VIP | NM_003381.4 | CACNB2 | NM_000724.4 |
| NRXN3 | NM_004796.6 | ECSCR | NM_001077693.4 |
| KDR | NM_002253.3 | GJA5 | NM_005266.6 |
| SMAD6 | NM_005585.5 | ENPP2 | NM_006209.5 |
| KCNAB1 | NM_172160.2 | CASQ2 | NM_001232.4 |
| CALCRL | NM_005795.6 | PTPRB | NM_001109754.4 |
| NOTCH4 | NM_004557.4 | TBX2 | NM_005994.4 |
| AQP1 | NM_198098.4 | ATP1A2 | NM_000702.4 |
| RAMP3 | NM_005856.3 | CD34 | NM_001025109.2 |
| HEY2 | NM_012259.3 | EDNRB | NM_000115.5 |

Exemplary amino acid sequences for various proteins encoded by genes described herein are provided below in Table 5. The amino acid sequences provided in Table 5 are for standard/wild-type versions of the listed proteins; different isoforms of the proteins and mutations thereof are also contemplated and within the scope of the embodiments provided herein.

TABLE 5
Amino acid sequences of proteins encoded by genes described herein

| Molecule | Amino Acid Sequence |
|---|---|
| CD163L1 | MMLPQNSWHIDFGRCCCHQNLFSAVVTCILLLNSCFLISSFNGTDLELRLVNGDGPCS |
| | GTVEVKFQGQWGTVCDDGWNTTASTVVCKQLGCPFSFAMFRFGQAVTRHGKIWLDD |
| | VSCYGNESALWECQHREWGSHNCYHGEDVGVNCYGEANLGLRLVDGNNSCSGRVE |
| | VKFQERWGTICDDGWNLNTAAVVCRQLGCPSSFISSGVVNSPAVLRPIWLDDILCQGN |
| | ELALWNCRHRGWGNHDCSHNEDVTLTCYDSSDLELRLVGGTNRCMGRVELKIQGRW |
| | GTVCHHKWNNAAADVVCKQLGCGTALHFAGLPHLQSGSDVVWLDGVSCSGNESFL |
| | WDCRHSGTVNFDCLHQNDVSVICSDGADLELRLADGSNNCSGRVEVRIHEQWWTICD |
| | QNWKNEQALVVCKQLGCPFSVFGSRRAKPSNEARDIWINSISCTGNESALWDCTYDG |
| | KAKRTCFRRSDAGVICSDKADLDLRLVGAHSPCYGRLEVKYQGEWGTVCHDRWSTR |
| | NAAVVCKQLGCGKPLHVFGMTYFKEASGPIWLDDVSCIGNESNIWDCEHSGWGKHN |
| | CVHREDVIVTCSGDATWGLRLVGGSNRCSGRLEVYFQGRWGTVCDDGWNSKAAAV |
| | VCSQLDCPSSIIGMGLGNASTGYGKIWLDDVSCDGDESDLWSCRNSGWGNNDCSHS |
| | EDVGVICSDASDMELRLVGGSSRCAGKVEVNVQGAVGILCANGWGMNIAEVVCRQLE |
| | CGSAIRVSREPHFTERTLHILMSNSGCTGGEASLWDCIRWEWKQTACHLNMEASLICS |
| | AHRQPRLVGADMPCSGRVEVKHADTWRSVCDSDFSLHAANVLCRELNCGDAISLSVG |
| | DHFGKGNGLTWAEKFQCEGSETHLALCPIVQHPEDTCIHSREVGVVCSRYTDVRLVN |
| | GKSQCDGQVEINVLGHWGSLCDTHWDPEDARVLCRQLSCGTALSTTGGKYIGERSV |
| | RVWGHRFHCLGNESLLDNCQMTVLGAPPCIHGNTVSVICTGSLTQPLFPCLANVSDPY |
| | LSAVPEGSALICLEDKRLRLVDGDSRCAGRVEIYHDGFWGTICDDGWDLSDAHVVCQ |
| | KLGCGVAFNATVSAHFGEGSGPIWLDDLNCTGMESHLWQCPSRGWGQHDCRHKED |
| | AGVICSEFTALRLYSETETESCAGRLEVFYNGTWGSVGRRNITTAIAGIVCRQLGCGEN |
| | GVVSLAPLSKTGSGFMWVDDIQCPKTHISIWQCLSAPWERRISSPAEETWITCEDRIRV |
| | RGGDTECSGRVEIWHAGSWGTVCDDSWDLAEAEVVCQQLGCGSALAALRDASFGQ |
| | GTGTIWLDDMRCKGNESFLWDCHAKPWGQSDCGHKEDAGVRCSGQSLKSLNASSG |
| | HLALILSSIFGLLLLVLFILFLTWCRVQKQKHLPLRVSTRRRGSLEENLFHEMETCLKRE |
| | DPHGTRTSDDTPNHGCEDASDTSLLGVLPASEATK (SEQ ID NO: 25) |
| DNMT1 | MPARTAPARVPTLAVPAISLPDDVRRRLKDLERDSLTEKECVKEKLNLLHEFLQTEIKN |
| | QLCDLETKLRKEELSEEGYLAKVKSLLNKDLSLENGAHAYNREVNGRLENGNQARSEA |
| | RRVGMADANSPPKPLSKPRTPRRSKSDGEAKRSRDPPASASQVTGIRAEPSPSPRITR |
| | KSTRQTTITSHFAKGPAKRKPQEESERAKSDESIKEEDKDQDEKRRRVTSRERVARPL |
| | PAEEPERAKSGTRTEKEEERDEKEEKRLRSQTKEPTPKQKLKEEPDREARAGVQADE |
| | DEDGDEKDEKKHRSQPKDLAAKRRPEEKEPEKVNPQISDEKDEDEKEEKRRKTTPKE |
| | PTEKKMARAKTVMNSKTHPPKCIQCGQYLDDPDLKYGQHPPDAVDEPQMLTNEKLSIF |
| | DANESGFESYEALPQHKLTCFSVYCKHGHLCPIDTGLIEKNIELFFSGSAKPIYDDDPSL |
| | EGGVNGKNLGPINEWWITGFDGGEKALIGFSTSFAEYILMDPSPEYAPIFGLMQEKIYIS |
| | KIVVEFLQSNSDSTYEDLINKIETTVPPSGLNLNRFTEDSLLRHAQFVVEQVESYDEAGD |
| | SDEQPIFLTPCMRDLIKLAGVTLGQRRAQARRQTIRHSTREKDRGPTKATTTKLVYQIF |
| | DTFFAEQIEKDDREDKENAFKRRRCGVCEVCQQPECGKCKACKDMVKFGGSGRSKQ |
| | ACQERRCPNMAMKEADDDEEVDDNIPEMPSPKKMHQGKKKKQNKNRISWVGEAVKT |
| | DGKKSYYKKVCIDAETLEVGDCVSVIPDDSSKPLYLARVTALWEDSSNGQMFHAHWF |
| | CAGTDTVLGATSDPLELFLVDECEDMQLSYIHSKVKVIYKAPSENWAMEGGMDPESLL |
| | EGDDGKTYFYQLWYDQDYARFESPPKTQPTEDNKFKFCVSCARLAEMRQKEIPRVLE |
| | QLEDLDSRVLYYSATKNGILYRVGDGVYLPPEAFTFNIKLSSPVKRPRKEPVDEDLYPE |
| | HYRKYSDYIKGSNLDAPEPYRIGRIKEIFCPKKSNGRPNETDIKIRVNKFYRPENTHKST |
| | PASYHADINLLYWSDEEAVVDFKAVQGRCTVEYGEDLPECVQVYSMGGPNRFYFLEA |
| | YNAKSKSFEDPPNHARSPGNKGKGKGKGKGKPKSQACEPSEPEIEIKLPKLRTLDVFS |
| | GCGGLSEGFHQAGISDTLWAIEMWDPAAQAFRLNNPGSTVFTEDCNILLKLVMAGETT |
| | NSRGQRLPQKGDVEMLCGGPPCQGFSGMNRFNSRTYSKFKNSLVVSFLSYCDYYRP |
| | RFFLLENVRNFVSFKRSMVLKLTLRCLVRMGYQCTFGVLQAGQYGVAQTRRRAIILAA |
| | APGEKLPLFPEPLHVFAPRACQLSVVVDDKKFVSNITRLSSGPFRTITVRDTMSDLPEV |
| | RNGASALEISYNGEPQSWFQRQLRGAQYQPILRDHICKDMSALVAARMRHIPLAPGSD |
| | WRDLPNIEVRLSDGTMARKLRYTHHDRKNGRSSSGALRGVCSCVEAGKACDPAARQ |
| | FNTLIPWCLPHTGNRHNHWAGLYGRLEWDGFFSTTVTNPEPMGKQGRVLHPEQHRV |
| | VSVRECARSQGFPDTYRLFGNILDKHRQVGNAVPPPLAKAIGLEIKLCMLAKARESASA |
| | KIKEEEAAKD (SEQ ID NO: 26) |
| MC1R | MAVQGSQRRLLGSLNSTPTAIPQLGLAANQTGARCLEVSISDGLFLSLGLVSLVENALV |
| | VATIAKNRNLHSPMYCFICCLALSDLLVSGSNVLETAVILLLEAGALVARAAVLQQLDNVI |
| | DVITCSSMLSSLCFLGAIAVDRYISIFYALRYHSIVTLPRARRAVAAIWVASVVFSTLFIAY |
| | YDHVAVLLCLVVFFLAMLVLMAVLYVHMLARACQHAQGIARLHKRQRPVHQGFGLKG |
| | AVTLTILLGIFFLCWGPFFLHLTLIVLCPEHPTCGCIFKNFNLFLALIICNAIIDPLIYAFHSQ |
| | ELRRTLKEVLTCSW (SEQ ID NO: 27) |
| FOXO1 | MAEAPQVVEIDPDFEPLPRPRSCTWPLPRPEFSQSNSATSSPAPSGSAAANPDAAAG |
| | LPSASAAAVSADFMSNLSLLEESEDFPQAPGSVAAAVAAAAAAAATGGLCGDFQGPE |
| | AGCLHPAPPQPPPPGPLSQHPPVPPAAAGPLAGQPRKSSSSRRNAWGNLSYADLITK |
| | AIESSAEKRLTLSQIYEWMVKSVPYFKDKGDSNSSAGWKNSIRHNLSLHSKFIRVQNE |
| | GTGKSSWWMLNPEGGKSGKSPRRRAASMDNNSKFAKSRSRAAKKKASLQSGQEGA |
| | GDSPGSQFSKWPASPGSHSNDDFDNWSTFRPRTSSNASTISGRLSPIMTEQDDLGEG |
| | DVHSMVYPPSAAKMASTLPSLSEISNPENMENLLDNLNLLSSPTSLTVSTQSSPGTMM |
| | QQTPCYSFAPPNTSLNSPSPNYQKYTYGQSSMSPLPQMPIQTLQDNKSSYGGMSQY |
| | NCAPGLLKELLTSDSPPHNDIMTPVDPGVAQPNSRVLGQNVMMGPNSVMSTYGSQA |
| | SHNKMMNPSSHTHPGHAQQTSAVNGRPLPHTVSTMPHTSGMNRLTQVKTPVQVPLP |
| | HPMQMSALGGYSSVSSCNGYGRMGLLHQEKLPSDLDGMFIERLDCDMESIIRNDLMD |
| | GDTLDFNFDNVLPNQSFPHSVKTTTHSWVSG (SEQ ID NO: 28) |
| STAB2 | MMLQHLVIFCLGLVVQNFCSPAETTGQARRCDRKSLLTIRTECRSCALNLGVKCPDGY |
| | TMITSGSVGVRDCRYTFEVRTYSLSLPGCRHICRKDYLQPRCCPGRWGPDCIECPGG |
| | AGSPCNGRGSCAEGMEGNGTCSCQEGFGGTACETCADDNLFGPSCSSVCNCVHGV |
| | CNSGLDGDGTCECYSAYTGPKCDKPIPECAALLCPENSRCSPSTEDENKLECKCLPNY |
| | RGDGKYCDPINPCLRKICHPHAHCTYLGPNRHSCTCQEGYRGDGQVCLPVDPCQINF |
| | GNCPTKSTVCKYDGPGQSHCECKEHYQNFVPGVGCSMTDICKSDNPCHRNANCTTV |
| | APGRTECICQKGYVGDGLTCYGNIMERLRELNTEPRGKWQGRLTSFISLLDKAYAWPL |
| | SKLGPFTVLLPTDKGLKGFNVNELLVDNKAAQYFVKLHIIAGQMNIEYMNNTDMFYTLT |
| | GKSGEIFNSDKDNQIKLKLHGGKKKVKIIQGDIIASNGLLHILDRAMDKLEPTFESNNEQT |
| | IMTMLQPRYSKFRSLLEETNLGHALDEDGVGGPYTIFVPNNEALNNMKDGTLDYLLSP |
| | EGSRKLLELVRYHIVPFTQLEVATLISTPHIRSMANQLIQFNTTDNGQILANDVAMEEIEIT |
| | AKNGRIYTLTGVLIPPSIVPILPHRCDETKREMKLGTCVSCSLVYWSRCPANSEPTALFT |
| | HRCVYSGRFGSLKSGCARYCNATVKIPKCCKGFYGPDCNQCPGGFSNPCSGNGQCA |
| | DSLGGNGTCICEEGFQGSQCQFCSDPNKYGPRCNKKCLCVHGTCNNRIDSDGACLT |
| | GTCRDGSAGRLCDKQTSACGPYVQFCHIHATCEYSNGTASCICKAGYEGDGTLCSEM |
| | DPCTGLTPGGCSRNAECIKTGTGTHTCVCQQGWTGNGRDCSEINNCLLPSAGGCHD |
| | NASCLYVGPGQNECECKKGFRGNGIDCEPITSCLEQTGKCHPLASCQSTSSGVWSCV |
| | CQEGYEGDGFLCYGNAAVELSFLSEAAIFNRWINNASLQPTLSATSNLTVLVPSQQAT |
| | EDMDQDEKSFWLSQSNIPALIKYHMLLGTYRVADLQTLSSSDMLATSLQGNFLHLAKV |
| | DGNITIEGASIVDGDNAATNGVIHIINKVLVPQRRLTGSLPNLLMRLEQMPDYSIFRGYII |
| | QYNLANAIEAADAYTVFAPNNNAIENYIREKKVLSLEEDVLRYHVVLEEKLLKNDLHNG |
| | MHRETMLGFSYFLSFFLHNDQLYVNEAPINYTNVATDKGVIHGLGKVLEIQKNRCDNN |
| | DTTIIRGRCRTCSSELTCPFGTKSLGNEKRRCIYTSYFMGRRTLFIGCQPKCVRTVITRE |
| | CCAGFFGPQCQPCPGNAQNVCFGNGICLDGVNGTGVCECGEGFSGTACETCTEGKY |
| | GIHCDQACSCVHGRCNQGPLGDGSCDCDVGWRGVHCDNATTEDNCNGTCHTSANC |
| | LTNSDGTASCKCAAGFQGNGTICTAINACEISNGGCSAKADCKRTTPGRRVCTCKAGY |
| | TGDGIVCLEINPCLENHGGCDKNAECTQTGPNQAACNCLPAYTGDGKVCTLINVCLTK |
| | NGGCSEFAICNHTGQVERTCTCKPNYIGDGFTCRGSIYQELPKNPKTSQYFFQLQEHF |
| | VKDLVGPGPFTVFAPLSAAFDEEARVKDWDKYGLMPQVLRYHVVACHQLLLENLKLIS |
| | NATSLQGEPIVISVSQSTVYINNKAKIISSDIISTNGIVHIIDKLLSPKNLLITPKDNSGRILQ |
| | NLTTLATNNGYIKFSNLIQDSGLLSVITDPIHTPVTLFWPTDQALHALPAEQQDFLFNQD |
| | NKDKLKEYLKFHVIRDAKVLAVDLPTSTAWKTLQGSELSVKCGAGRDIGDLFLNGQTC |
| | RIVQRELLFDLGVAYGIDCLLIDPTLGGRCDTFTTFDASGECGSCVNTPSCPRWSKPK |
| | GVKQKCLYNLPFKRNLEGCRERCSLVIQIPRCCKGYFGRDCQACPGGPDAPCNNRGV |
| | CLDQYSATGECKCNTGFNGTACEMCWPGRFGPDCLPCGCSDHGQCDDGITGSGQC |
| | LCETGWTGPSCDTQAVLPAVCTPPCSAHATCKENNTCECNLDYEGDGITCTVVDFCK |
| | QDNGGCAKVARCSQKGTKVSCSCQKGYKGDGHSCTEIDPCADGLNGGCHEHATCK |
| | MTGPGKHKCECKSHYVGDGLNCEPEQLPIDRCLQDNGQCHADAKCVDLHFQDTTVG |
| | VFHLRSPLGQYKLTFDKAREACANEAATMATYNQLSYAQKAKYHLCSAGWLETGRVA |
| | YPTAFASQNCGSGVVGIVDYGPRPNKSEMWDVFCYRMKDVNCTCKVGYVGDGFSCS |
| | GNLLQVLMSFPSLTNFLTEVLAYSNSSARGRAFLEHLTDLSIRGTLFVPQNSGLGENET |
| | LSGRDIEHHLANVSMFFYNDLVNGTTLQTRLGSKLLITASQDPLQPTETRFVDGRAILQ |
| | WDIFASNGIIHVISRPLKAPPAPVTLTHTGLGAGIFFAIILVTGAVALAAYSYFRINRRTIGF |
| | QHFESEEDINVAALGKQQPENISNPLYESTTSAPPEPSYDPFTDSEERQLEGNDPLRTL |
| | (SEQ ID NO: 29) |
| LOC728763 | MRAAIERRWRREQELCLQLKSSQALVASLQEQLSESRRELWAAQKLQQERAREQAR |
| | EREALRGQLEAQRLEVQQCRASCKLLGREKAALEMVVEELKGKADAADAEKQGLEAE |
| | AAELQRSLLLQAERREELALRRERSCRALETSQGRLQQLEEKVSGLREELASVREALS |
| | TAQLQRDVVESEREGLRSALARAECSNADLELLVRRLKSEGVEQRDSLAAMAALMEG |
| | LAQDKSALNHLALQLEQERDQLREQRKTLEQERARAGEQLAQAEQQLALERAERRGL |
| | QQACGRLEQRQEQLEGQAALLGREKAQLQEQVGQVTCQKQALEEQLAQSLQDQEA |
| | QMGTLQQALQGKDALSEERAQLLAKQEALERQGRLAAEEAADLRVERDSLESSLLEA |
| | QQLATKLQEQLEEEARSAGLARQALQGAPRALPQLLPRKSAMEGPLEWSSVPTLEME |
| | QLQSDWEVQEMKLRQDTVRLQRQVAQQEREAQRALESQALAHREALAQLQREKETL |
| | SLTLAEEKEVARCQLEQEKELVTKSAAEREALKGEIQSLKQERDESLLQLEHKMQQVM |
| | ALSLKETERSLLSEELSRARRTLERVQQEAQSQQEQAQRGLLWSQCWLLEPSLELAL |
| | SLPRTRYQACQPAGLPGELVKGSFATSAQKMEIKTVERSGRRGMPAKEHQAPDDRTA |
| | NTKPALEGAIPHQPPPASRLLLPGGLATISATTEELKALQAQFEDAITAHQRETTALRES |
| | LQDLAAERGDVEREAERLRAQLTVAQEGLAALRQELQGVEESREGLHREAQEARRAL |
| | SDEAREKDVLLLFNSELRATICRAEQEKASFKRSKEEKEQKLLILEEAQAALQQEASAL |
| | RAHLWELEQAGGDARQELRELHRQVRTLKAENQRRSGEAHELQAQCSQEVLELRRQ |
| | AAKAEAKHEGARKEVLGLQRKLAEVEAAGEAHGQRLQEHLRESRGAEQTLRAELHSV |
| | TRKLQEASGVADALQARLDQACHRIHSLEQELAQAEGARQDAEAQLGRLCSTLRRGL |
| | GLQRQSPWASPEQPGSPTKGLTTHTVRDSDRQL (SEQ ID NO: 30) |
| MYH7B | MSGNKRGSRASCPHRGAECLLPWAALNLQGFQLLLLHPSATAMMDVSELGESARYLR |
| | QGYQEMTKVHTIPWDGKKRVWVPDEQDAYVEAEVKSEATGGRVTVETKDQKVLMVR |
| | EAELQPMNPPRFDLLEDMAMMTHLNEASVLHNLRQRYARWMIYTYSGLFCVTINPYK |
| | WLPVYTASVVAAYKGKRRSDSPPHIYAVADNAYNDMLRNRDNQSMLITGESGAGKTV |
| | NTKRVIQYFAIVAALGDGPGKKAQFLATKTGGTLEDQIIEANPAMEAFGNAKTLRNDNS |
| | SRFGKFIRIHFGPSGKLASADIDSYLLEKSRVIFQLPGERSYHVYYQILSGRKPELQDML |
| | LLSMNPYDYHFCSQGVITVDNMNDGEELIATDHAMDILGFSVDEKCACYKIVGALLHFG |
| | NMKFKQKQREEQAEADGTESADKAAYLMGVSSGDLLKGLLHPRVRVGNEYVTKGQS |
| | VEQVVFAVGALAKATYDRLFRWLVSRINQTLDTKLPRQFFIGVLDIAGFEIFEFNSFEQL |
| | CINFTNEKLQQFFNQHMFVLEQEEYKREGIDWVFIDFGLDLQPCIDLIEKPLGILSILEEE |
| | CMFPKASDASFRAKLYDNHAGKSPNFQQPRPDKKRKYQAHFEVVHYAGVVPYSIVG |
| | WLEKNKDPLNETVVPIFQKSQNRLLATLYENYAGSCSTEPPKSGVKEKRKKAASFQTV |
| | SQLHKENLNKLMTNLRATQPHFVRCIVPNENKTPGVMDAFLVLHQLRCNGVLEGIRICR |
| | QGFPNRLLYTDFRQRYRILNPSAIPDDTFMDSRKATEKLLGSLDLDHTQYQFGHTKVFF |
| | KAGLLGVLEELRDQRLAKVLTLLQARSRGRLMRLEYQRLLGGRDALFTIQWNIRAFNA |
| | VKNWSWMKLFFKMKPLLRSAQAEEELAALRAELRGLRGALAAAEAKRQELEETHVSIT |
| | QEKNDLALQLQAEQDNLADAEERCHLLIKSKVQLEGKVKELSERLEDEEEVNADLAAR |
| | RRKLEDECTELKKDIDDLELTLAKAEKEKQATENKVKNLTEEMAALDESVARLTKEKKA |
| | LQEAHQQALGDLQAEEDRVSALTKAKLRLEQQVEDLECSLEQEKKLRMDTERAKRKL |
| | EGDLKLTQESVADAAQDKQQLEEKLKKKDSELSQLSLRVEDEQLLGAQMQKKIKELQA |
| | RAEELEEELEAERAARARVEKQRAEAARELEELSERLEEAGGASAGQREGCRKREAE |
| | LGRLRRELEEAALRHEATVAALRRKQAEGAAELGEQVDSLQRVRQKLEKEKSELRME |
| | VDDLAANVETLTRAKASAEKLCRTYEDQLSEAKIKVEELQRQLADASTQRGRLQTESG |
| | ELSRLLEEKECLISQLSRGKALAAQSLEELRRQLEEESKAKSALAHAVQALRHDCDLLR |
| | EQHEEEAEAQAELQRLLSKANAEVAQWRSKYEADAIQRTEELEEAKKKLALRLQEAEE |
| | GVEAANAKCSSLEKAKLRLQTESEDVTLELERATSAAAALDKKQRHLERALEERRRQE |
| | EEMQRELEAAQRESRGLGTELFRLRHGHEEALEALETLKRENKNLQEEISDLTDQVSL |
| | SGKSIQELEKTKKALEGEKSEIQAALEEAEGALELEETKTLRIQLELSQVKAEVDRKLAE |
| | KDEECANLRRNHQRAVESLQASLDAETRARNEALRLKKKMEGDLNDLELQLGHATRQ |
| | ATEAQAATRLMQAQLKEEQAGRDEEQRLAAELHEQAQALERRASLLAAELEELRAALE |
| | QGERSRRLAEQELLEATERLNLLHSQNTGLLNQKKKLEADLAQLSGEVEEAAQERREA |
| | EEKAKKAITDAAMMAEELKKEQDTSAHLERMKKTLEQTVRELQARLEEAEQAALRGGK |
| | KQVQKLEAKVRELEAELDAEQKKHAEALKGVRKHERRVKELAYQAEEDRKNLARMQD |
| | LVDKLQSKVKSYKRQFEEAEQQANTNLAKYRKAQHELDDAEERADMAETQANKLRAR |
| | TRDALGPKHKE (SEQ ID NO: 31) |
| IL16 | MESHSRAGKSRKSAKFRSISRSLMLCNAKTSDDGSSPDEKYPDPFEISLAQGKEGIFH |
| | SSVQLADTSEAGPSSVPDLALASEAAQLQAAGNDRGKTCRRIFFMKESSTASSREKPG |
| | KLEAQSSNFLFPKACHQRARSNSTSVNPYCTREIDFPMTKKSAAPTDRQPYSLCSNRK |
| | SLSQQLDCPAGKAAGTSRPTRSLSTAQLVQPSGGLQASVISNIVLMKGQAKGLGFSIV |
| | GGKDSIYGPIGIYVKTIFAGGAAAADGRLQEGDEILELNGESMAGLTHQDALQKFKQAK |
| | KGLLTLTVRTRLTAPPSLCSHLSPPLCRSLSSSTCITKDSSSFALESPSAPISTAKPNYRI |
| | MVEVSLQKEAGVGLGIGLCSVPYFQCISGIFVHTLSPGSVAHLDGRLRCGDEIVEISDS |
| | PVHCLTLNEVYTILSHCDPGPVPIIVSRHPDPQVSEQQLKEAVAQAVENTKFGKERHQ |
| | WSLEGVKRLESSWHGRPTLEKEREKNSAPPHRRAQKVMIRSSSDSSYMSGSPGGSP |
| | GSGSAEKPSSDVDISTHSPSLPLAREPVVLSIASSRLPQESPPLPESRDSHPPLRLKKS |
| | FEILVRKPMSSKPKPPPRKYFKSDSDPQKSLEERENSSCSSGHTPPTCGQEARELLPL |
| | LLPQEDTAGRSPSASAGCPGPGIGPQTKSSTEGEPGWRRASPVTQTSPIKHPLLKRQ |
| | ARMDYSFDTTAEDPWVRISDCIKNLFSPIMSENHGHMPLQPNASLNEEEGTQGHPDG |
| | TPPKLDTANGTPKVYKSADSSTVKKGPPVAPKPAWFRQSLKGLRNRASDPRGLPDPA |
| | LSTQPAPASREHLGSHIRASSSSSSIRQRISSFETFGSSQLPDKGAQRLSLQPSSGEAA |
| | KPLGKHEEGRFSGLLGRGAAPTLVPQQPEQVLSSGSPAASEARDPGVSESPPPGRQP |
| | NQKTLPPGPDPLLRLLSTQAEESQGPVLKMPSQRARSFPLTRSQSCETKLLDEKTSKL |
| | YSISSQVSSAVMKSLLCLPSSISCAQTPCIPKEGASPTSSSNEDSAANGSAETSALDTG |
| | FSLNLSELREYTEGLTEAKEDDDGDHSSLQSGQSVISLLSSEELKKLIEEVKVLDEATLK |
| | QLDGIHVTILHKEEGAGLGFSLAGGADLENKVITVHRVFPNGLASQEGTIQKGNEVLSIN |
| | GKSLKGTTHHDALAILRQAREPRQAVIVTRKLTPEAMPDLNSSTDSAASASAASDVSVE |
| | STEATVCTVTLEKMSAGLGFSLEGGKGSLHGDKPLTINRIFKGAASEQSETVQPGDEIL |
| | QLGGTAMQGLTRFEAWNIIKALPDGPVTIVIRRKSLQSKETTAAGDS (SEQ ID NO: 32) |
| SPATA31C2 | MENLPFPLKLLSASSLNTPSSTPWVLDIFLTLVFALGFFFLLLPYFSYLRCDNPPSPSPK |
| | KRKRHLVSQRPAGRRGRPRGRMKNHSLRACRECPRGLEETWDLLSQLQSLLGPHLE |
| | KGDFGQLSGPDPPGEVGKRTPDGASRSSHEPTEDAAPIVSPLASPDPRTKHPQDLAS |
| | TPPPGPMTTSVSSLSASQPPEPSLLLEHPSPEPPALFPHPPRTPDPLACSPPPPKGFT |
| | PPPLRDSTLLTPSHCDSVALPLDTVPQSLSPREDLAASVPGISGLGGSNSQVSALSWS |
| | QETTKTWCVFNSSVQQDHLSRQRDTTMSPLLFQAQPLSHLEPESQPFISSTPQFWPT |
| | PMAQAEAQAHLQSSFPVLSPAFLSPMKNTGVACPASQNKVQALSLPETQHPERPLLK |
| | KQLEGGLALPSRVQKSQDVFSVSTPNLPQERLTSILPENFPVSPELWRQLEQHMGQR |
| | GRIQESLDLMQLQDELPGTSQAKGKPRPWQSSTSTGESSKEAQTVKFQLERDPCPHL |
| | GQILGETPQNLSRGMESFPGKVLGATSEESERNLRKPLRSDSGSDLLRRTERNHIENIL |
| | KAHMSRKLGQTNEGLIPVSVRRSWLAVNQAFPVSNTHVKTSNLAAPKSRKACVNTAQ |
| | VLSFLEPCTQQVLGAHIVRFWAKHRWGLPLRVLKPIQCFQLEKVSSLSLIQLAGPSSDT |
| | CESGAGSKVEVATFLGEPPMASLRKQVLTKPSVHMPERLQASSPACKQFQRAPRGIP |
| | SSNDHGSLKAPTAGQEGRWPSKPLTYSLTGSTQQSRSLGAQSSRAGETREAVPQPT |
| | VPLGTCMRANLQATSEDVRGFKAPGASKSSLLPRMSVSQDPRKLCLMEEAVSEFEPG |
| | KATKSETQPQVSATVVLLPDGQASVVPHASENLASQVPQGHLQSMPTGNMQASQEL |
| | CDLMSARRSNMGHKEPRNPNCQGSCKSQSPMFPPTHKRENSRKPNLEKHEEMFQG |
| | LRTPQLTPGRKTEDTRQNEGVQLLPSKKQPPSISHFGENIKQFFQTIFSKKERKPAPVT |
| | AESQKTVKNRSCVYGSSAEAERLMTAVGQILEENMSLCHARHASKVNQQRQQFQAP |
| | VCGFPCNHRHPFYSEHSRMLSYAASSQQATLKNQSRPNRDRQIRDQ (SEQ ID NO: 33) |
| ARVCF1 | MEDCNVHSAASILASVKEQEARFERLTRALEQERRHVALQLERAQQPGMVSGGMGS |
| | GQPLPMAWQQLVLQEQSPGSQASLATMPEAPDVLEETVTVEEDPGTPTSHVSIVTSE |
| | DGTTRRTETKVTKTVKTVTTRTVRQVPVGPDGLPLLDGGPPLGPFADGALDRHFLLRG |
| | GGPVATLSRAYLSSGGGFPEGPEPRDSPSYGSLSRGLGMRPPRAGPLGPGPGDGCF |
| | TLPGHREAFPVGPEPGPPGGRSLPERFQAEPYGLEDDTRSLAADDEGGPELEPDYGT |
| | ATRRRPECGRGLHTRAYEDTADDGGELADERPAFPMVTAPLAQPERGSMGSLDRLV |
| | RRSPSVDSARKEPRWRDPELPEVLAMLRHPVDPVKANAAAYLQHLCFENEGVKRRVR |
| | QLRGLPLLVALLDHPRAEVRRRACGALRNLSYGRDTDNKAAIRDCGGVPALVRLLRAA |
| | RDNEVRELVTGTLWNLSSYEPLKMVIIDHGLQTLTHEVIVPHSGWEREPNEDSKPRDA |
| | EWTTVFKNTSGCLRNVSSDGAEARRRLRECEGLVDALLHALQSAVGRKDTDNKSVEN |
| | CVCIMRNLSYHVHKEVPGADRYQEAEPGPLGSAVGSQRRRRDDASCFGGKKAKEEW |
| | FHQGKKDGEMDRNFDTLDLPKRTEAAKGFELLYQPEVVRLYLSLLTESRNFNTLEAAA |
| | GALQNLSAGNWMWATYIRATVRKERGLPVLVELLQSETDKVVRAVAIALRNLSLDRRN |
| | KDLIGSYAMAELVRNVRNAQAPPRPGACLEEDTVVAVLNTIHEIVSDSLDNARSLLQAR |
| | GVPALVALVASSQSVREAKAASHVLQTVWSYKELRGTLQKDGWTKARFQSAAATAKG |
| | PKGALSPGGFDDSTLPLVDKSLEGEKTGSRDVIPMDALGPDGYSTVDRRERRPRGAS |
| | SAGEASEKEPLKLDPSRKAPPPGPSRPAVRLVDAVGDAKPQPVDSWV (SEQ ID NO: 34) |
| ABCA1 | MACWPQLRLLLWKNLTFRRRQTCQLLLEVAWPLFIFLILISVRLSYPPYEQHECHFPNK |
| | AMPSAGTLPWVQGIICNANNPCFRYPTPGEAPGVVGNFNKSIVARLFSDARRLLLYSQ |
| | KDTSMKDMRKVLRTLQQIKKSSSNLKLQDFLVDNETFSGFLYHNLSLPKSTVDKMLRA |
| | DVILHKVFLQGYQLHLTSLCNGSKSEEMIQLGDQEVSELCGLPREKLAAAERVLRSNM |
| | DILKPILRTLNSTSPFPSKELAEATKTLLHSLGTLAQELFSMRSWSDMRQEVMFLTNVN |
| | SSSSSTQIYQAVSRIVCGHPEGGGLKIKSLNWYEDNNYKALFGGNGTEEDAETFYDNS |
| | TTPYCNDLMKNLESSPLSRIIWKALKPLLVGKILYTPDTPATRQVMAEVNKTFQELAVFH |
| | DLEGMWEELSPKIWTFMENSQEMDLVRMLLDSRDNDHFWEQQLDGLDWTAQDIVAF |
| | LAKHPEDVQSSNGSVYTWREAFNETNQAIRTISRFMECVNLNKLEPIATEVWLINKSME |
| | LLDERKFWAGIVFTGITPGSIELPHHVKYKIRMDIDNVERTNKIKDGYWDPGPRADPFE |
| | DMRYVWGGFAYLQDVVEQAIIRVLTGTEKKTGVYMQQMPYPCYVDDIFLRVMSRSMP |
| | LFMTLAWIYSVAVIIKGIVYEKEARLKETMRIMGLDNSILWFSWFISSLIPLLVSAGLLVVIL |
| | KLGNLLPYSDPSVVFVFLSVFAVVTILQCFLISTLFSRANLAAACGGIIYFTLYLPYVLCVA |
| | WQDYVGFTLKIFASLLSPVAFGFGCEYFALFEEQGIGVQWDNLFESPVEEDGFNLTTS |
| | VSMMLFDTFLYGVMTWYIEAVFPGQYGIPRPWYFPCTKSYWFGEESDEKSHPGSNQK |
| | RISEICMEEEPTHLKLGVSIQNLVKVYRDGMKVAVDGLALNFYEGQITSFLGHNGAGKT |
| | TTMSILTGLFPPTSGTAYILGKDIRSEMSTIRQNLGVCPQHNVLFDMLTVEEHIWFYARL |
| | KGLSEKHVKAEMEQMALDVGLPSSKLKSKTSQLSGGMQRKLSVALAFVGGSKVVILDE |
| | PTAGVDPYSRRGIWELLLKYRQGRTIILSTHHMDEADVLGDRIAIISHGKLCCVGSSLFL |
| | KNQLGTGYYLTLVKKDVESSLSSCRNSSSTVSYLKKEDSVSQSSSDAGLGSDHESDTL |
| | TIDVSAISNLIRKHVSEARLVEDIGHELTYVLPYEAAKEGAFVELFHEIDDRLSDLGISSY |
| | GISETTLEEIFLKVAEESGVDAETSDGTLPARRNRRAFGDKQSCLRPFTEDDAADPND |
| | SDIDPESRETDLLSGMDGKGSYQVKGWKLTQQQFVALLWKRLLIARRSRKGFFAQIVL |
| | PAVFVCIALVFSLIVPPFGKYPSLELQPWMYNEQYTFVSNDAPEDTGTLELLNALTKDP |
| | GFGTRCMEGNPIPDTPCQAGEEEWTTAPVPQTIMDLFQNGNWTMQNPSPACQCSSD |
| | KIKKMLPVCPPGAGGLPPPQRKQNTADILQDLTGRNISDYLVKTYVQIIAKSLKNKIWVN |
| | EFRYGGFSLGVSNTQALPPSQEVNDAIKQMKKHLKLAKDSSADRFLNSLGRFMTGLDT |
| | KNNVKVWFNNKGWHAISSFLNVINNAILRANLQKGENPSHYGITAFNHPLNLTKQQLSE |
| | VALMTTSVDVLVSICVIFAMSFVPASFVVFLIQERVSKAKHLQFISGVKPVIYWLSNFVW |
| | DMCNYVVPATLVIIIFICFQQKSYVSSTNLPVLALLLLLYGWSITPLMYPASFVFKIPSTAY |
| | VVLTSVNLFIGINGSVATFVLELFTDNKLNNINDILKSVFLIFPHFCLGRGLIDMVKNQAM |
| | ADALERFGENRFVSPLSWDLVGRNLFAMAVEGVVFFLITVLIQYRFFIRPRPVNAKLSPL |
| | NDEDEDVRRERQRILDGGGQNDILEIKELTKIYRRKRKPAVDRICVGIPPGECFGLLGV |
| | NGAGKSSTFKMLTGDTTVTRGDAFLNKNSILSNIHEVHQNMGYCPQFDAITELLTGRE |
| | HVEFFALLRGVPEKEVGKVGEWAIRKLGLVKYGEKYAGNYSGGNKRKLSTAMALIGGP |
| | PVVFLDEPTTGMDPKARRFLWNCALSVVKEGRSVVLTSHSMEECEALCTRMAIMVNG |
| | RFRCLGSVQHLKNRFGDGYTIVVRIAGSNPDLKPVQDFFGLAFPGSVLKEKHRNMLQY |
| | QLPSSLSSLARIFSILSQSKKRLHIEDYSVSQTTLDQVFVNFAKDQSDDDHLKDLSLHKN |
| | QTVVDVAVLTSFLQDEKVKESYV (SEQ ID NO: 35) |
| PTEN | MTAIIKEIVSRNKRRYQEDGFDLDLTYIYPNIIAMGFPAERLEGVYRNNIDDVVRFLDSKH |
| | KNHYKIYNLCAERHYDTAKFNCRVAQYPFEDHNPPQLELIKPFCEDLDQWLSEDDNHV |
| | AAIHCKAGKGRTGVMICAYLLHRGKFLKAQEALDFYGEVRTRDKKGVTIPSQRRYVYY |
| | YSYLLKNHLDYRPVALLFHKMMFETIPMFSGGTCNPQFVVCQLKVKIYSSNSGPTRRE |
| | DKFMYFEFPQPLPVCGDIKVEFFHKQNKMLKKDKMFHFWVNTFFIPGPEETSEKVENG |
| | SLCDQEIDSICSIERADNDKEYLVLTLTKNDLDKANKDKANRYFSPNFKVKLYFTKTVEE |
| | PSNPEASSSTSVTPDVSDNEPDHYRYSDTTDSDPENEPFDEDQHTQITKV (SEQ ID NO: 36) |

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Throughout this specification and claims, “mg/kg” will be understood to mean mg administered per kg of body weight.

Exemplary methods and materials are described herein, although methods and materials similar or equivalent to those described herein can also be used in the practice or testing of the invention. The materials, methods, and examples are illustrative only and not intended to be limiting.

### II. METHODS, USES AND MEDICAMENTS

In one aspect of the invention, the invention provides a method, medicament or kit of parts for treating a cancer in, or improving progression free survival of, a patient comprising, for, or related to, administering to the patient a combination therapy which comprises a PD-1 axis binding antagonist and a VEGF pathway inhibitor, wherein the cancer in the patient is pre-determined to contain a protein altering genetic mutation in a gene selected from the group consisting of CD163L1, DNMT1, MCR1R, FOXO1, STAB2, LOC728763, MYH7B, IL16, SPATA31C2, ARVCF, and ABCA1. In some embodiments, the cancer in the patient does not, or is pre-determined not to, contain a protein altering mutation in the PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLK5, PTPN13, RTL1, SCAP, SHROOM2, SLCO2B1, TBX2, TENM3, TNRC6A, TTC28, USP42, ZC3H3, EFCAB6, MAP3K6, or PTPDC1 gene.

The combination therapy may also comprise one or more therapeutic agent, such as a chemotherapeutic or chemoradio therapy.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phil1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-im idazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy of the invention may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Each small molecule therapeutic agent in a combination therapy of the invention can be administered orally or parenterally, including the intravenous, intramuscular, intraperitoneal, subcutaneous, rectal, topical, and transdermal routes of administration.

A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is treatment-naïve. In other embodiments, the combination therapy is administered to a patient who failed to achieve a sustained response after prior therapy with a biotherapeutic or chemotherapeutic agent, i.e., is treatment-experienced.

A combination therapy of the invention is typically used to treat a tumor that is large enough to be found by palpation or by imaging techniques well known in the art, such as MRI, ultrasound, or CAT scan. In some embodiments, a combination therapy of the invention is used to treat an advanced stage tumor having dimensions of at least about 200 mm³, 300 mm³, 400 mm³, 500 mm³, 750 mm³, or up to 1000 mm³.

In some embodiments, a combination therapy of the invention is administered to a human patient who has a cancer that tests positive for PD-L1 expression. In some embodiments, PD-L1 expression can be detected using a diagnostic anti-human PD-L1 antibody, or antigen binding fragment thereof, in an IHC assay on an FFPE or frozen tissue section of a tumor sample removed from the patient. Typically, the patient's physician would order a diagnostic test to determine PD-L1 expression in a tumor tissue sample removed from the patient prior to initiation of treatment with the PD-1 axis binding antagonist and VEGF pathway inhibitor, but it is envisioned that the physician could order the first or subsequent diagnostic tests at any time after initiation of treatment, such as for example after completion of a treatment cycle.

Biotherapeutic agents in a combination therapy of the invention may be administered by continuous infusion, or by doses at intervals of, e.g., daily, every other day, three times per week, or one time each week, two weeks, three weeks, monthly, bimonthly, etc. A total weekly dose is generally at least 0.05 pg/kg, 0.2 pg/kg, 0.5 pg/kg, 1 pg/kg, 10 pg/kg, 100 pg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (2003) Cancer Immunol. Immunother. 52:133-144.

In some embodiments that employ an anti-human PD-1 mAb or anti-human PD-L1 mAb as the PD-1 axis binding antagonist in the combination therapy, the dosing regimen will comprise administering the mAb at a dose of about 1, 2, 3, 5, 10, 15 or 20 mg/kg body weight, or at a dose of about 50, 80, 100, 120, 150, 180, 200, 250, 300, 400, 800, 1200 mg flat dose at intervals of about 14 days (±2 days), about 21 days (±2 days), about 28 days (±2 days), about 30 days (±2 days), about 35 days (±2 days), or about 42 days (±2 days) throughout the course of treatment.

In some embodiments that employ an anti-human PD-1 mAb or anti-human PD-1 mAb as the PD-1 axis binding antagonist in the combination therapy, the dosing regimen will comprise administering the mAb at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In certain embodiments, a subject will be administered an intravenous (IV) infusion of a medicament comprising any of the PD-1 axis binding antagonist described herein.

In some embodiments, the PD-1 axis binding antagonist is avelumab or a biosimilar version thereof, which is administered intravenously at a dose selected from the group consisting of: 10 mg Q2W, 10 mg Q3W, 800 mg Q2W and 1200 mg Q3W.

In some embodiments of the invention, the PD-1 axis binding antagonist is pembrolizumab (aka MK-3475) or a biosimilar version thereof, which is administered at a dose selected from the group consisting of 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, 10 mg Q3W, or a flat-dose equivalents of any of these doses, i.e., such as 200 mg Q3W. In some embodiments, MK-3475 is administered at a dose of 400 mg Q6W (400 mg flat dose every six weeks). In some embodiments, MK-3475 or a biosimilar version thereof, is administered at a dose of 200 mg Q2W for adults and 2 mg/kg (up to 200 mg) Q3W for children. In some embodiments, MK-3475 is administered as a liquid medicament which comprises 25 mg/ml MK-3475, 7% (w/v) sucrose, 0.02%) (w/v) polysorbate 80 in 10 mM histidine buffer pH 5.5, and the selected dose of the medicament is administered by IV infusion over a time period of about 30 minutes. In some embodiments, MK-3475 is administered subcutaneously in a high concentration formulation, as described in US. Pat. No. 9,220,776, the disclosure of which is herein incorporated by reference in its entirety.

In some embodiments, the PD-1 axis binding antagonist is nivolumab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W, or a flat does equivalent of any of the forgoing doses, such as 240 mg Q2W.

In some embodiments, the PD-1 axis binding antagonist is atezolizumab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 15 mg/kg Q2W 1 mg/kg Q3W, 2 mg/kg Q3W, 3 mg/kg Q3W, 5 mg/kg Q3W, and 10 mg Q3W, 15 mg/kg Q3W or a flat does equivalent of any of the forgoing doses, such as 1200 mg Q3W. in some embodiments, atezolizumab or a biosimilar version thereof is administered as an IV infusion over 60 minutes. In some embodiments, atezolizumab or a biosimilar version thereof is administered subcutaneously.

In some embodiments, the PD-1 axis binding antagonist is durvalumab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 15 mg/kg Q2W 1 mg/kg Q3W, 2 mg/kg Q3W, 3 mg/kg Q3W, 5 mg/kg Q3W, and 10 mg Q3W, 15 mg/kg Q3W or a flat does equivalent of any of the forgoing doses. In some embodiments, durvalumab or a biosimilar version thereof is administered at a dose of 10 mg/kg Q2W and as an IV infusion over 60 minutes. In some embodiments, durvalumab or a biosimilar version thereof is administered subcutaneously.

In some embodiments, the PD-1 axis binding antagonist is cemiplimab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 15 mg/kg Q2W 1 mg/kg Q3W, 2 mg/kg Q3W, 3 mg/kg Q3W, 5 mg/kg Q3W, and 10 mg Q3W, 15 mg/kg Q3W or a flat does equivalent of any of the forgoing doses. In some embodiments, durvalumab or a biosimilar version thereof is administered at a dose of 350 mg Q2W and as an IV infusion over 30 minutes. In some embodiments, cemiplimab or a biosimilar version thereof is administered subcutaneously.

In some embodiments, a treatment cycle begins with the first day of combination treatment and last for 2 weeks. In such embodiments, the combination therapy is preferably administered for at least 12 weeks (6 cycles of treatment), more preferably at least 24 weeks, and even more preferably at least 2 weeks after the patient achieves a CR.

In some embodiments, the patient is selected for treatment with the combination therapy of the invention is the patient has been diagnosed with advanced RCC with predominantly clear cell subtype, and the primary tumor has been resected. In some embodiments, the patient has not received prior systemic therapy for advanced RCC.

The present invention also provides a medicament which comprises a PD-1 axis binding antagonist as described above and a pharmaceutically acceptable excipient. When the PD-1 binding antagonist is a biotherapeutic agent, e.g., a mAb, the antagonist may be produced in CHO cells using conventional cell culture and recovery/purification technologies.

In some embodiments, a medicament comprising an anti-PD-1 antibody or anti-PD-L1 antibody as the PD-1 axis binding antagonist may be provided as a liquid formulation or prepared by reconstituting a lyophilized powder with sterile water for injection prior to use.

The present invention also provides a medicament which comprises axitinib and a pharmaceutically acceptable excipient.

The PD-1 axis binding antagonist and VEGF pathway inhibitor medicaments described herein may be provided as a kit which comprises a first container and a second container and a package insert. The first container contains at least one dose of a medicament comprising PD-1 axis binding antagonist, the second container contains at least one dose of a medicament comprising a VEGF pathway inhibitor, and the package insert, or label, which comprises instructions for treating a patient for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. In some embodiments of the kit, the PD-1 axis binding antagonist is an anti-PD-L1 antibody and the instructions state that the medicaments are intended for use in treating a patient having a cancer that tests positive for PD-L1 expression by an IHC assay.

These and other aspects of the invention, including the exemplary specific embodiments listed below, will be apparent from the teachings contained herein.

Exemplary embodiments provided herein included the embodiments (E) as provided below:
E1. A method for treating cancer in a patient, wherein the cancer in the patient is pre-determined
   (a) to contain one or more protein altering mutations in one or more gene(s) selected from the group consisting of CD163L1, DNMT1, MC1R, FOXO1, STAB2, LOC728763, MYH7B, IL16, SPATA31C2, ARVCF, and ABCA1, and/or
   (b) to not contain a protein altering mutation in one or more gene(s) selected from the group consisting of the PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLK5, PTPN13, RTL1, SCAP, SHROOM2, SLCO2B1, TBX2, TENM3, TNRC6A, TTC28, USP42, ZC3H3, EFCAB6, MAP3K6, and PTPDC1;
   comprising administering to the patient a therapeutically effective amount of a PD-1 axis binding antagonist.
E2. The method of embodiment 1, further comprising administering to the patient a therapeutically effective amount of a VEGF pathway inhibitor.
E3. The method of embodiment 2, wherein the VEGF pathway inhibitor is a VEGFR inhibitor.
E4. The method of embodiment 2, wherein the VEGF pathway inhibitor is axitinib or a pharmaceutically acceptable salt thereof.
E5. The method of any one of embodiments 1 to 4, wherein the pre-determination of the genetic mutation comprises a genetic testing of a tumor tissue sample of the cancer patient.
E6. The method of any one of embodiments 1 to 5, wherein the cancer in the patient is pre-determined to contain a protein altering mutation of the CD163L1 gene.
E7. The method of embodiment 6, wherein the protein altering mutation is a mutation listed in Table 1.1.
E8. The method of any one of embodiments 1 to 5, wherein the cancer in the patient is pre-determined to contain a protein altering mutation of the DNMT1 gene.
E9. The method of embodiment 8, wherein the protein altering mutation is a mutation listed in Table 1.2.
E10. The method of any one of embodiments 1 to 5, wherein the cancer in the patient is pre-determined to contain a protein altering mutation of the MC1R gene.
E11. The method of embodiment 10, wherein the protein altering mutation is a mutation listed in Table 1.3.
E12. The method of any one of embodiments 1 to 5, wherein the cancer in the patient is pre-determined to contain a protein altering mutation in one or more genes selected from the group consisting of CD163L1, DNMT1 and MC1R.
E13. The method of any one of embodiments 1 to 12, wherein the PD-1 axis binding antagonist is an anti-PD-1 antibody.
E14. The method of embodiment 13, wherein the anti-PD-1 antibody is selected from the group consisting of pembrolizumab, nivolumab, cemiplimab and RN888.
E15. The method of embodiment 14, wherein the anti-PD-1 antibody comprises
   (a) a full length heavy chain having an amino acid sequence of SEQ ID NO: 9, and a full length light chain having an amino acid sequence of SEQ ID NO: 10;
   (b) a full length heavy chain having an amino acid sequence of SEQ ID NO: 7, and a full length light chain having an amino acid sequence of SEQ ID NO:8; or
   (c) a heavy chain variable region (VH) having an amino acid sequence of SEQ ID NO:5, and a light chain variable region (VL) of an amino acid sequence of SEQ ID NO:4.
E16. The method of any one of embodiments 1 to 12, wherein the PD-1 axis binding antagonist is an anti-PD-L1 antibody.
E17. The method of embodiment 16, wherein the anti-PD-L1 antibody is selected from the group consisting of avelumab, atezolizumab and durvalumab.
E18. The method of embodiment 16, wherein the anti-PD-L1 antibody comprises
   (a) a VH having an amino acid sequence of SEQ ID NO:14, and a VL having an amino acid sequences of SEQ ID NO:15; or
   (b) a VH having an amino acid sequence of SEQ ID NO:12, and a VL having an amino acid sequence of SEQ ID NO:13.
E19. The method of any one of embodiments 1 to 18, wherein the VEGF pathway inhibitor is axitinib or a pharmaceutically acceptable salt thereof.
E20. The method of any one of embodiments 1 to 19, wherein the PD-1 axis binding antagonist is administered at a dose of about 5 mg/kg, about 10 mg/kg, about 200 mg, about 240 mg, about 800 mg or about 1200 mg, and is administered about once a week, or about once every two, three, four, five weeks or six weeks; and the VEGF pathway inhibitor is administered at a dose of about 3 mg/kg, about 5 mg/kg, or about 5 mg and is administered twice daily.
E21. A medicament comprising a PD-1 axis binding antagonist for use in treating a cancer in a patient, wherein the cancer of the patient is pre-determined as
   (a) containing one or more protein altering mutations in one or more gene(s) selected from the group consisting of CD163L1, DNMT1, MC1R, FOXO1, STAB2, LOC728763, MYH7B, IL16, SPATA31C2, ARVCF, and ABCA1, and/or
   (b) not containing a protein altering mutation in one or more gene(s) selected from the group consisting of PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, and PTPDC1.
E22. The medicament of embodiment 21, wherein the medicament is to be used in combination with a VEGF pathway inhibitor.
E23. A kit which comprises a first container, a second container and a package insert, wherein the first container comprises at least one dose of a medicament comprising an PD-1 axis binding antagonist, the second container comprises at least one dose of a medicament comprising a VEGF pathway inhibitor, and the package insert comprises instructions for treating a subject for cancer wherein the cancer is pre-determined as
   (a) containing one or more protein altering genetic mutations in one or more gene(s) selected from the group consisting of CD163L1, DNMT1, MC1R, FOXO1, STAB2, LOC728763, MYH7B, IL16, SPATA31C2, ARVCF, and ABCA1, and/or
   (b) not containing a protein altering genetic mutation in one or more gene(s) selected from the group consisting of PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, and PTPDC1.
E24. A method for improving progression free survival of a patient suffering from cancer comprising administering to the patient an effective amount of a PD-1 axis binding antagonist, wherein the cancer of the patient (a) contains one or more protein altering mutations in one or more genes selected from the group consisting of CD163L1, DNMT1, MC1R, FOXO1, STAB2, LOC728763, MYH7B, IL16, SPATA31C2, ARVCF, and ABCA1, and/or (b) does not contain a protein altering mutation in one or more gene(s) selected from the group consisting of PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, and PTPDC1.
E25. The method of embodiment 24, further comprising administering to the patient an effective amount of a VEGF pathway inhibitor.
E26. The method of embodiment 24 or 25, wherein the progression free survival of the patient is improved as compared to patients suffering from the cancer but whose cancer does not contain the protein altering mutation in (a) or contains a protein altering mutation in (b).
E27. The method, medicament for use, or kit of any one of embodiments 1 to 26, wherein the cancer is advanced or metastatic solid tumor.
E28. The method, medicament for use, or kit of any one of embodiments 1 to 27, wherein the cancer is bladder cancer, breast cancer, clear cell kidney cancer, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), liver cancer, mantle cell lymphoma (MCL), multiple myeloma (MM), myelodysplastic syndrome (MDS), non-Hodgkin's lymphoma (NHL), Squamous Cell Carcinoma of the Head and Neck (SCCHN), or small lymphocytic lymphoma (SLL).
E29. The method, medicament for use, or kit of any one of embodiments 1 to 28, wherein the cancer of the patient (a) contains one or more protein altering mutations in one or more genes selected from the group consisting of CD163L1, DNMT1, MC1R, FOXO1, STAB2, LOC728763, MYH7B, IL16, SPATA31C2, ARVCF, and ABCA1, and (b) does not contain a protein altering mutation in one or more gene(s) selected from the group consisting of PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, and PTPDC1.
E30. The method, medicament for use, or kit of any one of embodiments 1 to 29, wherein the cancer of the patient contains one or more protein altering mutations in two or more genes selected from the group consisting of CD163L1, DNMT1, MC1R, FOXO1, STAB2, LOC728763, MYH7B, IL16, SPATA31C2, ARVCF, and ABCA1.
E31. The method, medicament for use, or kit of any one of embodiments 1 to 30, wherein the cancer of the patient contains one or more protein altering mutations in one or more genes selected from the group consisting of FOXO1, IL16, and SPATA31C2, and wherein the mutation is a germline mutation.
E32. A method of treating a patient having a cancer, comprising administering to the patient a therapeutically effective amount of a PD-1 axis binding antagonist, wherein the expression level of the gene UTS2 in a sample obtained from the patient has been determined to be increased as compared to a reference level.
E33. A method of treating a patient having a cancer, comprising administering to the patient a therapeutically effective amount of a PD-1 axis binding antagonist, wherein the expression level of at least one gene selected from the group consisting of

CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCL5, XCL2, CST7, GFI1, KCNA3, PSTPIP1 in a sample obtained from the patient has been determined to be increased as compared to a reference level.
E34. The method of embodiment 33, wherein the expression level of at least 2, 3, 4, 5, 6, 7, 8, 10, 15, 20, or 25 genes selected from the group have been determined to be increased as compared to a reference level.
E35. The method of embodiment 33, wherein the expression level of all 26 of the genes of the group have been determined to be increased as compared to a reference level.
E36. The method of any one of embodiments 32-35, further comprising administering to the patient a therapeutically effective amount of a VEGF pathway inhibitor.
E37. The method of embodiment 36, wherein the VEGF pathway inhibitor is a VEGFR inhibitor.
E38. The method of embodiment 37, wherein the VEGF pathway inhibitor is axitinib or a pharmaceutically acceptable salt thereof.
E39. The method of any one of embodiments 32 to 38, wherein the PD-1 axis binding antagonist is an anti-PD-1 antibody.
E40. The method of embodiment 39, wherein the anti-PD-1 antibody is selected from the group consisting of pembrolizumab, nivolumab, cemiplimab and RN888.
E41. The method of embodiment 40, wherein the anti-PD-1 antibody comprises
   (a) a full length heavy chain having an amino acid sequence of SEQ ID NO: 9, and a full length light chain having an amino acid sequence of SEQ ID NO: 10;
   (b) a full length heavy chain having an amino acid sequence of SEQ ID NO: 7, and a full length light chain having an amino acid sequence of SEQ ID NO:8; or
   (c) a heavy chain variable region (VH) having an amino acid sequence of SEQ ID NO:5, and a light chain variable region (VL) of an amino acid sequence of SEQ ID NO:4.
E42. The method of any one of embodiments 32 to 38, wherein the PD-1 axis binding antagonist is an anti-PD-L1 antibody.
E43. The method of embodiment 42, wherein the anti-PD-L1 antibody is selected from the group consisting of avelumab, atezolizumab and durvalumab.
E44. The method of embodiment 43, wherein the anti-PD-L1 antibody comprises
   (a) a VH having an amino acid sequence of SEQ ID NO:14, and a VL having an amino acid sequences of SEQ ID NO:15; or
   (b) a VH having an amino acid sequence of SEQ ID NO:12, and a VL having an amino acid sequence of SEQ ID NO:13.
E45. The method of any one of embodiments 36 to 44, wherein the VEGF pathway inhibitor is axitinib or a pharmaceutically acceptable salt thereof.
E46. The method of any one of embodiments 32 to 45, wherein the PD-1 axis binding antagonist is administered at a dose of about 5 mg/kg, about 10 mg/kg, about 200 mg, about 240 mg, about 800 mg or about 1200 mg, and is administered about once a week, or about once every two, three, four, five weeks or six weeks; and the VEGF pathway inhibitor is administered at a dose of about 3 mg/kg, about 5 mg/kg, or about 5 mg and is administered twice daily.
E47. A method of identifying a patient having a cancer who may benefit from a treatment comprising a therapeutically effective amount of a PD-1 axis binding antagonist, the method comprising determining an expression level of the gene UTS2 in a sample obtained from the patient, wherein an increased expression level of UTS2 in the sample as compared to a reference level identifies the patient as one who has an increased likelihood of benefiting from a treatment comprising a therapeutically effective amount of a PD-1 axis binding antagonist.
E48. A method of predicting responsiveness of a patient having a cancer to a treatment comprising a therapeutically effective amount of a PD-1 axis binding antagonist, the method comprising determining an expression level of the gene UTS2 in a sample obtained from the patient, wherein an increased expression level of UTS2 in the sample as compared to a reference level indicates that the patient has an increased likelihood of benefiting from a treatment comprising a therapeutically effective amount of a PD-1 axis binding antagonist.
E49. A method of identifying a patient having a cancer who may benefit from a treatment comprising a therapeutically effective amount of a PD-1 axis binding antagonist, the method comprising determining an expression level of at least one gene selected from the group consisting of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1 in a sample obtained from the patient, wherein an increased expression level of the at least one gene in the sample as compared to a reference level identifies the patient as one who has an increased likelihood of benefiting from a treatment comprising a therapeutically effective amount of a PD-1 axis binding antagonist.
E50. A method of predicting responsiveness of a patient having a cancer to a treatment comprising a therapeutically effective amount of a PD-1 axis binding antagonist, the method comprising determining an expression level of at least one gene selected from the group consisting of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1 in a sample obtained from the patient, wherein an increased expression level of the at least one gene in the sample as compared to a reference level indicates that the patient has an increased likelihood of benefiting from a treatment comprising a therapeutically effective amount of a PD-1 axis binding antagonist.
E51. The method of embodiment 49 or 50, wherein the expression level of at least 2, 3, 4, 5, 6, 7, 8, 10, 15, 20, or 25 genes selected from the group have been determined to be increased as compared to a reference level, and wherein an increased expression level of the at least 2, 3, 4, 5, 6, 7, 8, 10, 15, 20, or 25 genes in the sample as compared to a reference level indicates that the patient has an increased likelihood of benefiting from a treatment comprising a therapeutically effective amount of a PD-1 axis binding antagonist.
E52. The method of embodiment 49 or 50, wherein the expression level of all 26 of the genes of the group have been determined to be increased as compared to a reference level, and wherein an increased expression level of all 26 of the genes in the sample as compared to a reference level indicates that the patient has an increased likelihood of benefiting from a treatment comprising a therapeutically effective amount of a PD-1 axis binding antagonist.
E53. The method of any one of embodiments 47 to 52, wherein an increased expression level of UTS2 or an increased expression level at least 1, 2, 3, 4, 5, 6, 7, 8, 10, 15, 20, 25 or 26 genes selected from the group consisting of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1 as compared to a reference level identifies the patient as one who has an increased likelihood of benefiting from a treatment comprising a therapeutically effective amount of a PD-1 axis binding antagonist and which further comprises a therapeutically effective amount of a VEGF pathway inhibitor.
E54. The method of embodiment 53, wherein the VEGF pathway inhibitor is a VEGFR inhibitor.
E55. The method of embodiment 54, wherein the VEGF pathway inhibitor is axitinib or a pharmaceutically acceptable salt thereof.
E56. The method of any one of embodiments 47 to 55, wherein the PD-1 axis binding antagonist is an anti-PD-1 antibody.
E57. The method of embodiment 56, wherein the anti-PD-1 antibody is selected from the group consisting of pembrolizumab, nivolumab, cemiplimab and RN888.
E58. The method of embodiment 57, wherein the anti-PD-1 antibody comprises
   (a) a full length heavy chain having an amino acid sequence of SEQ ID NO: 9, and a full length light chain having an amino acid sequence of SEQ ID NO: 10;
   (b) a full length heavy chain having an amino acid sequence of SEQ ID NO: 7, and a full length light chain having an amino acid sequence of SEQ ID NO:8; or
   (c) a heavy chain variable region (VH) having an amino acid sequence of SEQ ID NO:5, and a light chain variable region (VL) of an amino acid sequence of SEQ ID NO:4.
E59. The method of any one of embodiments 47 to 55, wherein the PD-1 axis binding antagonist is an anti-PD-L1 antibody.
E60. The method of embodiment 59, wherein the anti-PD-L1 antibody is selected from the group consisting of avelumab, atezolizumab and durvalumab.
E61. The method of embodiment 60, wherein the anti-PD-L1 antibody comprises
   (a) a VH having an amino acid sequence of SEQ ID NO:14, and a VL having an amino acid sequences of SEQ ID NO:15; or
   (b) a VH having an amino acid sequence of SEQ ID NO:12, and a VL having an amino acid sequence of SEQ ID NO:13.
E62. The method of any one of embodiments 53 to 61, wherein the VEGF pathway inhibitor is axitinib or a pharmaceutically acceptable salt thereof.
E63. The method of any one of embodiments 53 to 62, wherein the PD-1 axis binding antagonist is administered at a dose of about 5 mg/kg, about 10 mg/kg, about 200 mg, about 240 mg, about 800 mg or about 1200 mg, and is administered about once a week, or about once every two, three, four, five weeks or six weeks; and the VEGF pathway inhibitor is administered at a dose of about 3 mg/kg, about 5 mg/kg, or about 5 mg and is administered twice daily.
E64. A method of identifying a patient having a cancer who may benefit from a treatment comprising a therapeutically effective amount of a PD-1 axis binding antagonist, the method comprising determining an expression level of the gene DUX4 or the DUX4 gene signature in a sample obtained from the patient, wherein an increased expression level of the gene DUX4 or the DUX4 gene signature in the sample as compared to a reference level identifies the patient as one who has a decreased likelihood of benefiting from a treatment comprising a therapeutically effective amount of a PD-1 axis binding antagonist.
E65. A method of predicting responsiveness of a patient having a cancer to a treatment comprising a therapeutically effective amount of a PD-1 axis binding antagonist, the method comprising determining an expression level of the gene DUX4 or the DUX4 gene signature in a sample obtained from the patient, wherein an increased expression level of the gene DUX4 or the DUX4 gene signature in the sample as compared to a reference level indicates that the patient has a decreased likelihood of benefiting from a treatment comprising a therapeutically effective amount of a PD-1 axis binding antagonist.
E66. A method of identifying a patient having a cancer who may benefit from a treatment comprising a therapeutically effective amount of a PD-1 axis binding antagonist, the method comprising determining an expression level of at least 1, 2, 3, 4, 5, 6, 7, 8, 10, 15, 20, 25 or 26 genes selected from the group consisting of NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB in a sample obtained from the patient, wherein an increased expression level of the at least 1, 2, 3, 4, 5, 6, 7, 8, 10, 15, 20, 25 or 26 genes in the sample as compared to a reference level identifies the patient as one who has a decreased likelihood of benefiting from a treatment comprising a therapeutically effective amount of a PD-1 axis binding antagonist.
E67. A method of predicting responsiveness of a patient having a cancer to a treatment comprising a therapeutically effective amount of a PD-1 axis binding antagonist, the method comprising determining an expression level of at least 1, 2, 3, 4, 5, 6, 7, 8, 10, 15, 20, 25 or 26 genes selected from the group consisting of NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB in a sample obtained from the patient, wherein an increased expression level of the at least 1, 2, 3, 4, 5, 6, 7, 8, 10, 15, 20, 25 or 26 genes in the sample as compared to a reference level indicates that the patient has a decreased likelihood of benefiting from a treatment comprising a therapeutically effective amount of a PD-1 axis binding antagonist.
E68. The method of any one of embodiments 66 or 67 wherein an increased expression level at least 1, 2, 3, 4, 5, 6, 7, 8, 10, 15, 20, 25 or 26 genes selected from the group consisting of NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB as compared to a reference level further identifies the patient as one who has an increased likelihood of benefiting from a treatment comprising a therapeutically effective amount of a VEGF pathway inhibitor.
E69. The method of embodiment 68, wherein the VEGF pathway inhibitor is a VEGFR inhibitor.
E70. The method of embodiment 69, wherein the VEGF pathway inhibitor is sunitinib or a pharmaceutically acceptable salt thereof.
E71. A medicament comprising a PD-1 axis binding antagonist for use in treating a cancer in a patient, wherein a sample from the patient is pre-determined to have at least one of and optionally two, three, four, five, six, or all seven of the following characteristics:
   (i) it contains one or more protein altering mutations in one or more gene(s) selected from the group consisting of CD163L1, DNMT1, MC1R, FOXO1, STAB2, LOC728763, MYH7B, IL16, SPATA31C2, ARVCF, and ABCA1;
   (ii) it does not contain a protein altering mutation in one or more gene(s) selected from the group consisting of PTEN, ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, and PTPDC1;
   (iii) it has an increased expression level of the gene UTS2 as compared to a reference level;
   (iv) it has an increased expression level of at least one gene selected from the group consisting of at least 1, 2, 3, 4, 5, 6, 7, 8, 10, 15, 20, 25 of or all 26 genes selected from the group consisting of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1 as compared to a reference level;
   (v) it does not have an increased expression level of the gene DUX4 or a DUX4 gene signature as compared to a reference level;
   (vi) it does not have an increased expression level of at least one gene selected from the group consisting of at least 1, 2, 3, 4, 5, 6, 7, 8, 10, 15, 20, 25 of or all 26 genes selected from the group consisting of NRARP, RAMP2, ARHGEF15, VIP, NRXN3, KDR, SMAD6, KCNAB1, CALCRL, NOTCH4, AQP1, RAMP3, TEK, FLT1, GATA2, CACNB2, ECSCR, GJAS, ENPP2, CASQ2, PTPRB, TBX2, ATP1A2, CD34, HEY2, EDNRB as compared to a reference level; or
   (vii) it contains one or more protein altering mutations in the NOTCH2 gene.
E72. The medicament of embodiment 71, wherein the medicament is to be used in combination with a VEGF pathway inhibitor.
E73. The method or medicament of any one of embodiments 32 to 72, wherein the respective reference level of gene expression is determined based on an average level of the gene expression from a plurality of samples from patients having the cancer.
E74. The method or medicament of any one of embodiments 32 to 72, wherein the respective reference level of gene expression is determined based on an average level of the gene expression from a plurality of samples from human subjects.
E75. The method or medicament of any one of embodiments 32 to 72, wherein the respective reference level of gene expression is the level of gene expression of a reference gene in a cancer from the patient.
E76. The method or medicament of any one of embodiments 32 to 72, wherein the sample obtained from the patient is a tissue sample, a whole blood sample, a plasma sample, or a serum sample.
E77. The method or medicament of embodiment 76, wherein the tissue sample is a tumor tissue sample.
E78. The method or medicament of any one of embodiments 32 to 77, wherein the expression level is an mRNA expression level.
E79. The method or medicament of embodiment 78, wherein the mRNA expression level is determined by RNA sequencing, RT-PCR, gene expression profiling, serial analysis of gene expression, or microarray analysis.
E80. The method or medicament of any one of embodiments 32 to 77, wherein the expression level is a protein expression level.
E81. A kit which comprises a first container, a second container and a package insert, wherein the first container comprises at least one dose of a medicament comprising an PD-1 axis binding antagonist, the second container comprises at least one dose of a medicament comprising a VEGF pathway inhibitor, and the package insert comprises instructions for treating a subject for cancer wherein the cancer is pre-determined as having an increased expression level of the gene UTS2 and/or an increased expression level of 1, 2, 3, 4, 5, 6, 7, 8, 10, 15, 20, 25 or 26 genes selected from the group consisting of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCLS, XCL2, CST7, GFI1, KCNA3, PSTPIP1, as compared to a reference level of the respective gene(s).
E82. The method, medicament or kit of any one of embodiments 32 to 81, wherein cancer is advanced or metastatic solid tumor.
E83. The method, medicament or kit of any one of embodiments 32 to 82, wherein the cancer is bladder cancer, breast cancer, clear cell kidney cancer, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), liver cancer, mantle cell lymphoma (MCL), multiple myeloma (MM), myelodysplastic syndrome (MDS), non-Hodgkin's lymphoma (NHL), Squamous Cell Carcinoma of the Head and Neck (SCCHN), small lymphocytic lymphoma (SLL), endometrial cancer, B-cell acute lymphoblastic leukemia, colorectal cancer, glioblastoma, cervical cancer, penile cancer, or non-melanoma skin cancer.

### III. GENERAL METHODS

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols.1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, N.Y.; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, NJ; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

The presence and/or expression level (amount) of various biomarkers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, enzyme-linked immunosorbent assay (ELISA), enzyme-linked immunofiltration assay (ELIFA), fluorescence activated cell sorting (“FACS”), MassARRAY, proteomics, quantitative blood based assays (e.g., serum ELISA), biochemical enzymatic activity assays, in situ hybridization, fluorescence in situ hybridization (FISH), Southern analysis, Northern analysis, whole genome sequencing, polymerase chain reaction (PCR) (including quantitative real time PCR (qRT-PCR) and other amplification type detection methods, such as, for example, branched DNA, SISBA, TMA and the like), RNA-Seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

Incorporated by reference herein for all purposes is the content of U.S. Provisional Patent Application Nos. 62/855,923 (filed Jun. 1, 2019), 62/927,963 (filed Oct. 30, 2019) and 63/013,132 (filed Apr. 21, 2020).

### IV. EXAMPLES

### Example 1

### Combination Treatment with Avelumab and Axitinib

This example illustrates a clinical trial study (ClinicalTrials.gov Identifier: NCT02493751) to evaluate safety, efficacy, pharmacokinetics, and pharmacodynamics of avelumab (MSB0010718C) in combination with axitinib (AG-013736) in patients with previously untreated advanced renal cell carcinoma (aRCC).

This study is an open-label, multi-center, multiple-dose trial designed to estimate the maximum tolerated dose (MTD) and select the recommended phase 2 dose (RP2D) of avelumab (MSB0010718C) in combination with axitinib (AG-013736). Once the MTD of avelumab administered in combination with axitinib is estimated (dose finding portion), the dose expansion phase will be opened to further characterize the combination in term of safety profile, anti-tumor activity, pharmacokinetics, pharmacodynamics and biomarker modulation. Protocol design is set forth in Table 6.

The Dose Finding Phase will estimate the MTD and RP2D in patients with aRCC with clear cell histology who did not receive prior systemic therapy for advanced disease, using the modified toxicity probability interval (mTPI) method.35 Dose finding will follow an “Up-and-Down” design, with up to 4 potential dose levels (DL) to be tested, shown in Table 6.

The Dose Finding Phase will lead to the identification of an Expansion Test Dose for avelumab in combination with axitinib in patients with aRCC who did not receive prior systemic therapy for their advanced disease. The Expansion Test Dose will either be the MTD (i.e., the highest dose of avelumab and axitinib associated with the occurrence of DLTs in <33% of patients) or the RP2D, i.e., the highest tested dose that is declared safe and tolerable by the investigators and sponsor. Once the Expansion Test Dose is identified, the Dose Expansion Phase will be opened, and avelumab in combination with axitinib will be evaluated in up to approximately 20-40 patients with previously untreated aRCC.

TABLE 6

| Arms | Assigned Interventions |
|---|---|
| Dose | Group 1: avelumab 10 mg/kg IV Q2W; axitinib 5 mg oral BID |
| finding | Group 2: avelumab 5 mg/kg IV Q2W; axitinib 5 mg oral BID |
| phase | Group 3: avelumab 10 mg/kg IV Q2W; axitinib 3 mg oral BID |
| | Group 4: avelumab 5 mg/kg IV Q2W; axitinib 3 mg oral BID |
| Dose | Group 1: avelumab 10 mg/kg IV Q2W; axitinib 5 mg oral BID |
| expan- | Group 2: avelumab 5 mg/kg IV Q2W; axitinib 5 mg oral BID |
| sion | Group 3: avelumab 10 mg/kg IV Q2W; axitinib 3 mg oral BID |
| phase | Group 4: avelumab 5 mg/kg IV Q2W; axitinib 3 mg oral BID |

Inclusion Criteria: Histologically or cytologically confirmed advanced RCC with clear cell component. Primary tumor resected. Mandatory archival formalin fixed, paraffin embedded (FFPE) tumor tissue block from primary tumor resection specimen (all patients). For Extension Cohort only, mandatory de novo tumor biopsy from a locally recurrent or metastatic lesion unless obtained from a procedure performed within 6 months of study entry and if the patient has received no intervening systemic anti-cancer treatment. At least one measurable lesion as defined by RECIST version 1.1. Age years. Eastern Cooperative Oncology Group (ECOG) performance status 0 or 1. Adequate bone marrow function, renal and liver functions.

The number of patients to be enrolled in the Dose Finding Phase will depend on the observed safety profile, and the number of tested dose levels. Up to approximately 55 patients (including Dose Finding Phase and Dose Expansion Phase) are projected to be enrolled in the study.

Study Treatment: Axitinib will be given orally (PO) twice daily (BID), with or without food, on a continuous dosing schedule. Avelumab will be given as a 1-hour intravenous infusion (IV) every two weeks (Q2W). In all patients, treatment with study drugs may continue until confirmed disease progression, patient refusal, patient lost to follow up, unacceptable toxicity, or the study is terminated by the sponsor, whichever comes first.

In order to mitigate avelumab infusion-related reactions, a premedication regimen of 25 to 50 mg IV or oral equivalent diphenhydramine and 650 mg IV or oral equivalent acetaminophen/paracetamol (as per local practice) may be administered approximately 30 to 60 minutes prior to each dose of avelumab. This may be modified based on local treatment standards and guidelines, as appropriate.

Tumor Assessment: Anti-tumor activity will be assessed by radiological tumor assessments at 6-week intervals, using RECIST version 1.1. Complete and partial responses will be confirmed on repeated imaging at least at 4 weeks after initial documentation. After 1 year from enrollment in the study, tumor assessments should be conducted less frequently, i.e., at 12-week intervals. In addition, radiological tumor assessments will also be conducted whenever disease progression is suspected (e.g., symptomatic deterioration), and at the time of End of Treatment/Withdrawal (if not done in the previous 6 weeks). If radiologic imaging shows progressive disease (PD), tumor assessment should be repeated at least >4 weeks later in order to confirm PD.

Brain Computerized Tomography (CT) or Magnetic Resonance Imaging (MRI) scans are required at baseline and when there is a suspected brain metastasis. Bone scan (bone scintigraphy) or 18fluorodeoxyglucose-positron emission tomography/CT(18FDG-PET/CT) are required at baseline, then every 16 weeks only if bone metastases are present at baseline. Otherwise, bone imaging is required only if new bone metastases are suspected. Bone imaging is also required at the time of confirmation of CR for patients who have bone metastases.

Pharmacokinetic/Immunogenicity Assessments: PK/immunogenicity sampling will be collected. To understand the PK effects of avelumab on axitinib, a 7-day lead-in period with single-agent axitinib will be included prior to Cycle 1 in all patients in the Dose Finding Phase and in at least 8 patients in the Dose Expansion Phase of the study. Since avelumab has a long half-life (3-5 days), it would not be feasible to run a lead-in to study the PK of avelumab alone. Therefore, the effect of axitinib on avelumab will be evaluated by comparing avelumab trough concentrations at steady state in the presence of axitinib with those reported for avelumab alone in prior studies.

Biomarker Assessments: A key objective of the biomarker analyses that will be performed in this study is to investigate biomarkers that are potentially predictive of treatment benefit with the combination of avelumab and axitinib. In addition, biomarker studies of tumor and blood biospecimens will be carried out to help further understand the mechanism of action of the avelumab in combination with axitinib, as well as potential mechanisms of resistance.

Tumor biospecimens from archived tissue samples and metastatic lesions will be used to analyze candidate DNA, RNA, or protein markers, or a relevant signature of markers, for their ability to identify those patients who are most likely to benefit from treatment with the study drugs. Markers that may be analyzed include, but not be limited to, PD-L1 expression tumor-infiltrating CD8+ T lymphocytes, and T-cell receptor gene sequence quantitation. Optional tumor biopsies obtained upon disease progression will be used to investigate acquired mechanisms of resistance. Only core needle or excisional biopsies, or resection specimen are suitable.

Peripheral Blood: Specimens will be retained as whole blood, serum, and plasma in a biobank for exploratory biomarker assessments, unless prohibited by local regulation or by decision of the Institutional Review Board or Ethics Committee. Samples may be used to identify or characterize cells, DNA, RNA, or protein markers known or suspected to be of relevance to the mechanisms of action, or the development of resistance to avelumab used in combination with axitinib. These include biomarkers that may aid in the identification of those patients who might preferentially benefit from treatment with avelumab in combination with axitinib, including but not limited to biomarkers related to anti-tumor immune response or target modulation, such as soluble VEGF-A, IL-8, IFNγ and/or tissue FoxP3, PD-1, PD-L2. Biospecimens should be obtained pre-dose and at the same time as PK samples whenever possible.

Trial Results:

The MTD/RP2D for this expansion phase and further studies in aRCC has been confirmed as avelumab 10 mg/kg IV Q2W+axitinib 5 mg PO BID continuously. The regimen has shown preliminary antitumour activity in treatment-naïve pts with aRCC. Enrollment is ongoing in the expansion cohort. These results demonstrate the efficacy and safety of avelumab+axitinib vs current monotherapies for aRCC.

### Example 2

### Combination Treatment with Axitinib and Avelumab Versus Sunitinib

This example illustrates a phase 3 clinical trial study (ClinicalTrials.gov Identifier: NCT02684006) to evaluate safety and efficacy of avelumab (MSB0010718C) in combination with axitinib (AG-013736) and to demonstrate the superiority of this combination versus standard-of-care sunitinib monotherapy in the first-line treatment of patients with advanced RCC (aRCC). Sunitinib malate (SUTENT®) is an oral multitargeted TKI of stem cell receptor factor (KIT), platelet derived growth factor-receptors (PDGFRs), VEGFRs, glial cell-line neurotrophic factor receptor (RET), and FMS-like tyrosine kinase 3 (FLT3), and colony stimulating factor receptor Type 1 (CSR-1R) approved multinationally for the treatment of aRCC, imatinib-resistant or intolerant gastrointestinal stromal tumor (GIST), and unresectable, well-differentiated metastatic pancreatic neuroendocrine tumors (NET).

The study is a Phase 3, randomized, multination, multicenter, open-label, parallel 2-arm study in which approximately 465 patients are planned to be randomized to receive avelumab in combination with axitinib or sunitinib monotherapy: Arm A: avelumab in combination with axitinib; Arm B: sunitinib. Patients will be stratified according to ECOG performance status (0 versus 1) and LDH (>1.5 ULN vs. ≤1.5 ULN). In arm A (avelumab in combination with axitinib), avelumab will be given as a 1 hour intravenous infusion (IV) every 2 weeks in a 6-week cycle. Axitinib will be given orally (PO) twice daily (BID), with or without food, on a continuous dosing schedule.

Treatment with study drugs may continue until confirmed disease progression, patient refusal, patient lost to follow up, unacceptable toxicity, or the study is terminated by the sponsor, whichever comes first. Axitinib treatment may be adjusted by dosing interruption with or without dose reduction. Intrapatient axitinib dose escalation may occur if the intrapatient escalation criteria are met.

Study Treatment: Axitinib will be given orally twice daily PO on a continuous daily dosing schedule. Avelumab will be given as a 1 hour intravenous infusion every 2 weeks in a 6-week cycle. Sunitinib will be given orally 50 mg taken once daily, on a schedule 4 weeks on treatment followed by 2 weeks off (Schedule 4/2). Patients who develop disease progression on study treatment but are otherwise continuing to derive clinical benefit from study treatment will be eligible to continue with avelumab combined with axitinib, or single-agent avelumab, or single-agent axitinib, or single-agent sunitinib provided that the treating physician has determined that the benefit/risk for doing so is favorable.

Tumor Assessments: Anti-tumor activity will be assessed by radiological tumor assessments and will be based on RECIST guidelines version 1.1 for primary and secondary endpoints and on immune-related RECIST (irRECIST) guidelines for exploratory endpoints. Tumor assessments will be performed every 6 weeks (Q6W) up to 1 year from first dose therapy; thereafter, tumor assessments will be performed every 2 cycles. In addition, radiological tumor assessments will also be conducted whenever disease progression is suspected (e.g., symptomatic deterioration), at the time of the End of Treatment/Withdrawal visit (if not done in the previous 6 weeks), and during the Short term Follow-up period (at the 90-day visit only); subsequent tumor assessments during the Long term Follow-up period can be collected in absence of withdrawal of consent, regardless of initiation of subsequent anti-cancer therapies.

Tumor assessments will include all known or suspected disease sites. Imaging may include chest, abdomen, and pelvis CT or MRI scans; brain CT or MRI scans (required at baseline and when suspected brain metastasis) and bone scans or 18FDG PET (required at baseline then every 16 weeks only if bone metastases are present at baseline). Otherwise, bone imaging is required only if new bone metastasis are suspected and at the time of confirmation of complete response for patients who have bone metastases. The CT scans should be performed with contrast agents unless contraindicated for medical reasons. The same imaging technique used to characterize each identified and reported lesion at baseline will be employed in the following tumor assessments. Antitumor activity will be assessed through radiological tumor assessments conducted at baseline, at 6 weeks after the first dose of therapy, then every 6 weeks up to 1 year from the first dose of therapy and every 12 weeks thereafter, (if not done in the previous 6 weeks), and during the Short term Follow-up period (at the 90-day visit only); subsequent tumor assessments during the Long term Follow-up period can be collected in absence of withdrawal of consent, regardless of initiation of subsequent anti-cancer therapies. Further imaging assessments may be performed at any time if clinically indicated (e.g., suspected PD, symptomatic deterioration, etc.). Assessment of response will be made using RECIST version 1.1 and as per immune-related response criteria (irRC) (Nishino 2013). All radiographic images will be collected and may be objectively verified by a BICR independent third-party core imaging laboratory.

Primary Endpoint: Progression-Free Survival (PFS) as assessed by Blinded Independent Central Review (BICR) per RECIST v1.1. Secondary Endpoints: Overall Survival (OS); objective tumor response rate (OR), as assessed by BICR per RECIST version 1.1.; disease Control (DC), as assessed by BICR per RECIST version 1.1.; time to event: time to response (TTR), Duration of Response (DR); adverse Events (AEs) as characterized by type, frequency, severity (as graded by National Cancer Institute Common Terminology Criteria for Adverse Events (NCI CTCAE v.4.03), timing, seriousness, and relationship to study therapy; Laboratory abnormalities as characterized by type, frequency, severity (as graded by NCI CTCAE v.4.03), and timing; PK parameters including trough concentrations (Ctrough) of avelumab and trough concentrations (Ctrough) and maximum concentrations (Cmax) of axitinib; tumor tissue biomarker status (i.e., positive or negative; based on for example, PD-L1 expression and/or quantitation of tumor infiltrating CD8+ T lymphocytes as assessed by immunohistochemistry); measures of clinical outcome (PFS, OS, OR, DCR, DR and TTR) in biomarker-positive and biomarker-negative sub-groups; anti-drug antibodies (ADAs; neutralizing antibodies) of avelumab when in combination with axitinib; patient-Reported Outcomes (PRO): FACT-Kidney Symptom Index (FKSI-19), EuroQol 5 Dimension (EQ 5D).

Trial Results:

A total of 886 patients were assigned to receive avelumab plus axitinib (442 patients) or sunitinib (444 patients). Among the 560 patients with PD-L1—positive tumors (63.2%), the median progression-free survival was 13.8 months with avelumab plus axitinib, as compared with 7.2 months with sunitinib (hazard ratio for disease progression or death, 0.61; 95% confidence interval [CI], 0.47 to 0.79; P<0.001); in the overall population, the median progression-free survival was 13.8 months, as compared with 8.4 months (hazard ratio, 0.69; 95% CI, 0.56 to 0.84; P<0.001). Among the patients with PD-L1—positive tumors, the objective response rate was 55.2% with avelumab plus axitinib and 25.5% with sunitinib; at a median follow-up for overall survival of 11.6 months and 10.7 months in the two groups, 37 patients and 44 patients had died, respectively. The trial results were published New England Journal of Medicine entitled “Avelumab plus Axitinib versus Sunitinib for Advanced Renal-Cell Carcinoma” (R. J. Motzer et. Al, N EngL J. Med 380, 12, page 1103-1116, March 2019), the disclosure of which is herein incorporated by reference in its entirety.

### Example 3

### Biomarker Study and Results of Patients in the Phase 3 Study of Avelumab with Axitinib Versus Sunitinib in Advanced Renal Cell Carcinoma

This example illustrates biomarker studies and results from the clinical trial study described in Example 2 above.

Tumor samples were collected from patients in Example 2 and were subject to whole exome sequencing to identify genetic mutations. Software tools BWA and a combination of MuTect, Vardict, Picard, and other vendor tools were used. Mutation with a minimum of 5 mutant reads, i.e. found on at least 5 separate DNA samples in an individual tumor sample, not annotated as synonymous variants and annotated as resulting in a change in protein coding sequence, and with at least 5% variant allele frequency in the patient population, were included in the analysis.

The Cox proportional hazards (PH) regression model was used to assess the dependence of progression-free survival (PFS) on mutational status of each gene. In addition, multivariate analysis was also carried out adjusting for age, sex and tumor mutation burden. For each gene, four Cox PH models were constructed: (1) a univariate and (2) a multivariate model using the full cohort with an interaction term between mutational status and treatment groups; (3) a univariate and (4) a multivariate model using the Avelumab+Axitinib arm. Genes were filtered based on the following criteria:
A. Mutated at 5% or more in the B9991003 cohort
B. Interaction term P-value<0.05 in both model (1) and model (2), testing for treatment specific differences of the mutations on survival outcome.
C. P-value<0.05 in both model (3) and model (4), testing for significant effects the mutants have on survival outcome in the Avelumab+Axitinib arm.
D. Hazard ratio (HR) <1 in both model (3) and model (4), filtering for mutations that have a beneficial effect on survival outcome in the Avelumab+Axitinib arm.

Applying the above criteria A through D, we obtained 9 genes of interest: ABCA1, CD163L1, DNMT1, FOXO1, IL16, LOC728763, MYH7B, SPATA31C2, STAB2. Specific mutations identified in the tumor samples of this study are listed in Tables 1.1 to Table 1.11. After manual inspection and applying prior knowledge, we also included another gene MC1R which barely misses above criteria A but satisfies above criteria B, C, D. Further analysis identified the gene ARVCF as also satisfying criteria A-D. Table 7 describes the probability of progression free survival at 15 months, 20 months after initial treatment in patients with above genetic mutation comparing to who does not have such mutation.

TABLE 7
Rate of Progression Free Survival
in Patients with Genetic Mutations

| | Number of | Rate of PFS | Rate of PFS |
|---|---|---|---|
| Patient group | Patients | at 15 months | at 20 months |
| CDL163L1 mutation | 25 | 90% | 90% |
| Avelumab + axitinib arm, | | | |
| CDL163L1 wild type | 346 | 50% | 40% |
| Avelumab + axitinib arm | | | |
| CDL163L1 mutation | 22 | 15% | 15% |
| Sunitinib arm | | | |
| CDL163L1 wild type | 355 | 25% | 25% |
| Sunitinib arm | | | |
| DNMT1 mutation | 21 | 80% | 80% |
| Avelumab + axitinib arm, | | | |
| DNMT1 wild type | 350 | 45% | 40% |
| Avelumab + axitinib arm | | | |
| DNMT1 mutation | 16 | 0% | 0% |
| Sunitinib arm | | | |
| DNMT1 wild type | 361 | 25% | 25% |
| Sunitinib arm | | | |
| MCR1 mutation | 20 | 80% | 80% |
| Avelumab + axitinib arm, | | | |
| MCR1 wild type | 351 | 45% | 40% |
| Avelumab + axitinib arm | | | |
| MCR1 mutation | 15 | 30% | n/a |
| Sunitinib arm | | | |
| MCR1 wild type | 362 | 30% | 30% |
| Sunitinib arm | | | |
| ABCA1 mutation | 27 | 55% | 55% |
| Avelumab + axitinib arm, | | | |
| ABCA1 wild type | 344 | 40% | 40% |
| Avelumab + axitinib arm | | | |
| ABCA1 mutation | 23 | 25% | n/a |
| Sunitinib arm | | | |
| ABCA1 wild type | 354 | 25% | 25% |
| Sunitinib arm | | | |
| FOXO1 mutation | 32 | 75% | 75% |
| Avelumab + axitinib arm, | | | |
| FOXO1 wild type | 339 | 45% | 40% |
| Avelumab + axitinib arm | | | |
| FOXO1 mutation | 25 | 10% | 10% |
| Sunitinib arm | | | |
| FOXO1 wild type | 352 | 25% | 25% |
| Sunitinib arm | | | |
| IL16 mutation | 63 | 70% | 60% |
| Avelumab + axitinib arm, | | | |
| IL16 wild type | 308 | 40% | 35% |
| Avelumab + axitinib arm | | | |
| IL16 mutation | 53 | 25% | 25% |
| Sunitinib arm | | | |
| IL16 wild type | 324 | 23% | 23% |
| Sunitinib arm | | | |
| MYH7B mutation | 42 | 65% | 65% |
| Avelumab + axitinib arm, | | | |
| MYH7B wild type | 329 | 45% | 40% |
| Avelumab + axitinib arm | | | |
| MYH7B mutation | 38 | 10% | n/a |
| Sunitinib arm | | | |
| MYH7B wild type | 339 | 25% | 25% |
| Sunitinib arm | | | |
| STAB2 mutation | 30 | 75% | 75% |
| Avelumab + axitinib arm, | | | |
| STAB2 wild type | 341 | 45% | 33% |
| Avelumab + axitinib arm | | | |
| STAB2 mutation | 37 | 30% | 30% |
| Sunitinib arm | | | |
| STAB2 wild type | 340 | 25% | 25% |
| Sunitinib arm | | | |
| LOC728763 mutation | 39 | 75% | 75% |
| Avelumab + axitinib arm, | | | |
| LOC728763 wild type | 332 | 35% | 35% |
| Avelumab + axitinib arm | | | |
| LOC728763 mutation | 27 | 25% | 25% |
| Sunitinib arm | | | |
| LOC728763 wild type | 350 | 23% | 23% |
| Sunitinib arm | | | |
| SPATA31C2 mutation | 55 | 60% | 60% |
| Avelumab + axitinib arm, | | | |
| SPATA31C2 wild type | 316 | 35% | 35% |
| Avelumab + axitinib arm | | | |
| SPATA 31C2 mutation | 54 | 25% | 25% |
| Sunitinib arm | | | |
| SPATA31C2 wild type | 323 | 23% | 23% |
| Sunitinib arm | | | |
| ARVCF mutation | 20 | 71% | N/A |
| Avelumab + axitinib arm | | | |
| ARVCF wild type | 20 | 27% | N/A |
| Avelumab + axitinib arm | | | |
| ARVCF mutation | 346 | 43% | 39% |
| Sunitinib arm | | | |
| ARVCF wild type | 352 | 22% | 22% |
| Sunitinib arm | | | |
| PTEN mutation | 23 | 10% | n/a |
| Avelumab + axitinib arm, | | | |
| PTEN wild type | 348 | 45% | 45% |
| Avelumab + axitinib arm | | | |
| PTEN mutation | 34 | 20% | n/a |
| Sunitinib arm | | | |
| PTEN wild type | 343 | 25% | 25% |
| Sunitinib arm | | | |
| Double mutant of | 5 | 100% | 100% (at 18 |
| CD163L1, MC1R and | | | months) |
| DNMT1 Avelumab + | | | |
| axitinib arm, | | | |
| Single mutant of | 56 | 80% | 80% |
| CD163L1, MC1R and | | | |
| DNMT1 Avelumab + | | | |
| axitinib arm | | | |
| Wild type of CD163L1, | 310 | 35% | 35% |
| MC1R and NDMT1 | | | |
| Avelumab + axitinib arm | | | |
| Double mutant of | 2 | 0 | 0 |
| CD163L1, MC1R and | | | |
| NDMT1 Sunitinib arm | | | |
| Single mutant of | 49 | 15% | 15% (at 18 |
| CD163L1, MC1R and | | | month) |
| NDMT1 Sunitinib arm | | | |
| Wild type of | 326 | 25% | 25% |
| CD163L1, MC1R and | | | |
| NDMT1 Sunitinib arm | | | |

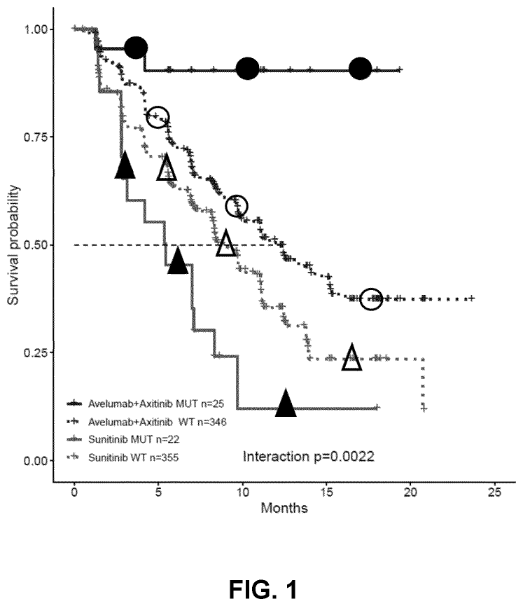

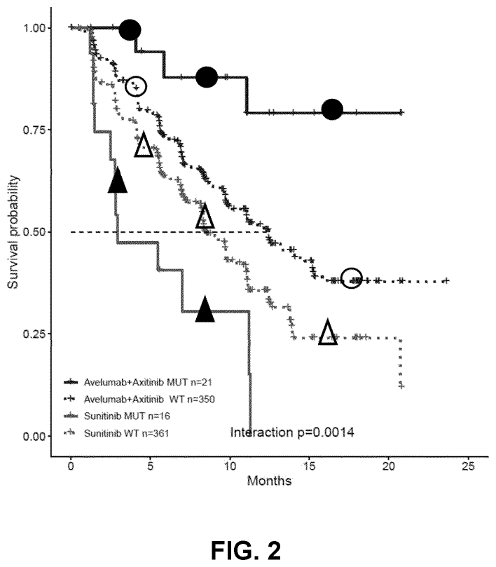

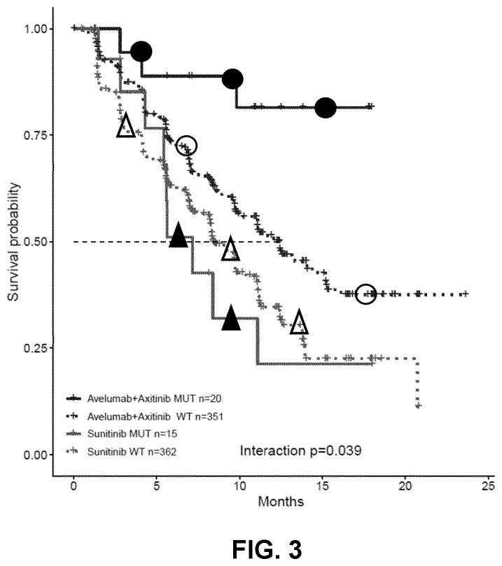

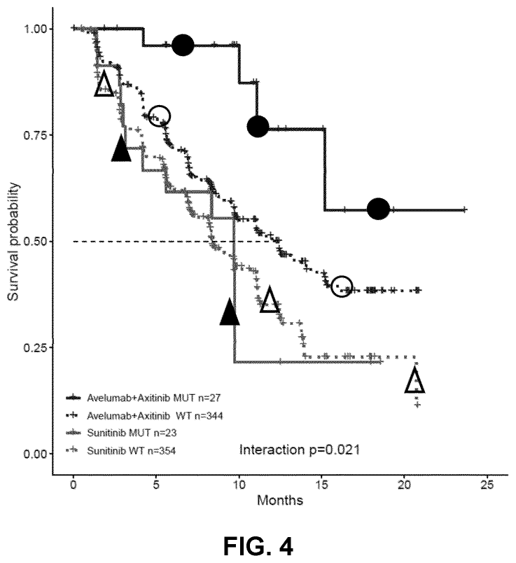

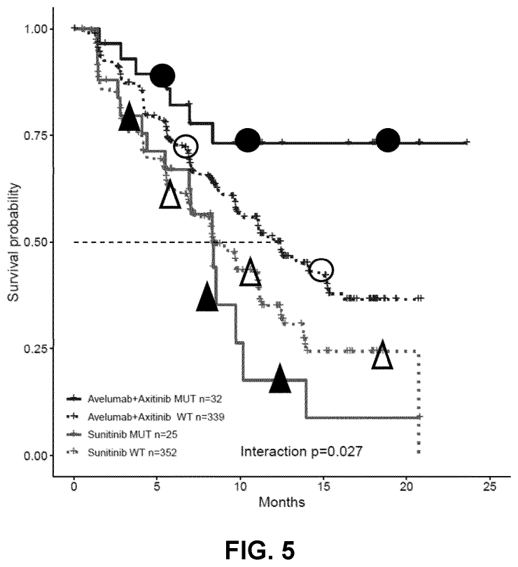

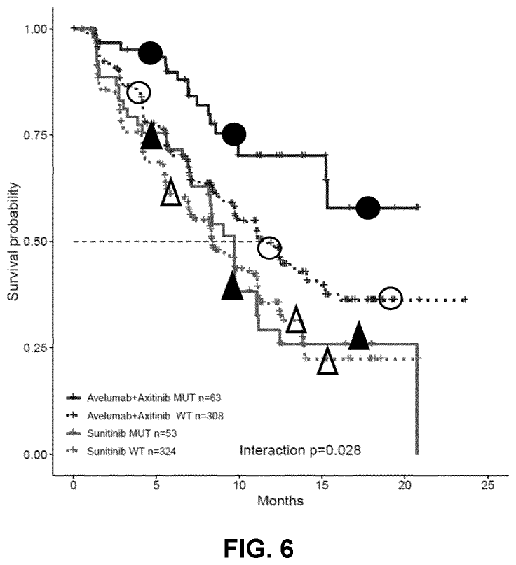

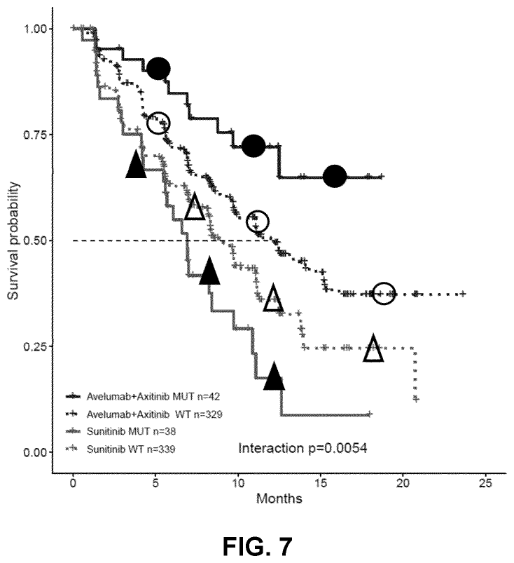

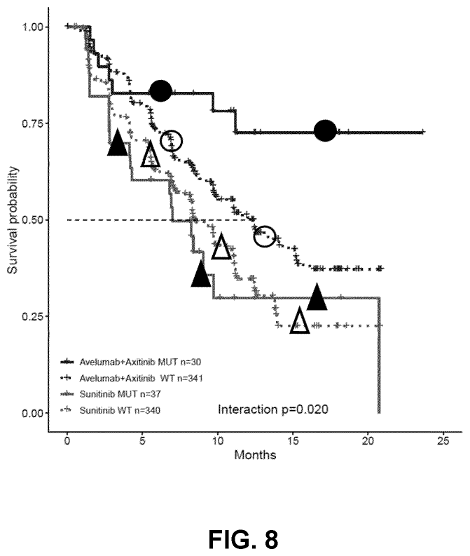

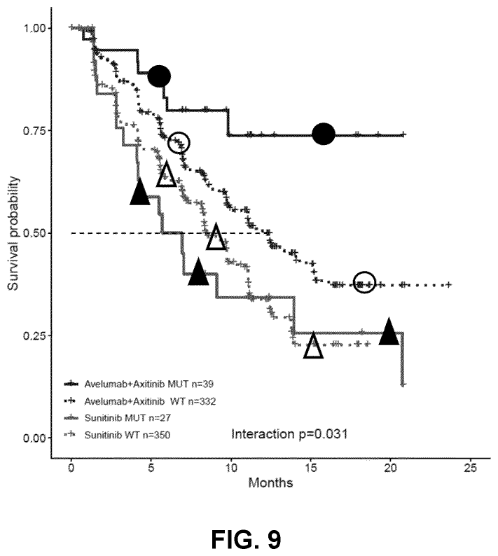

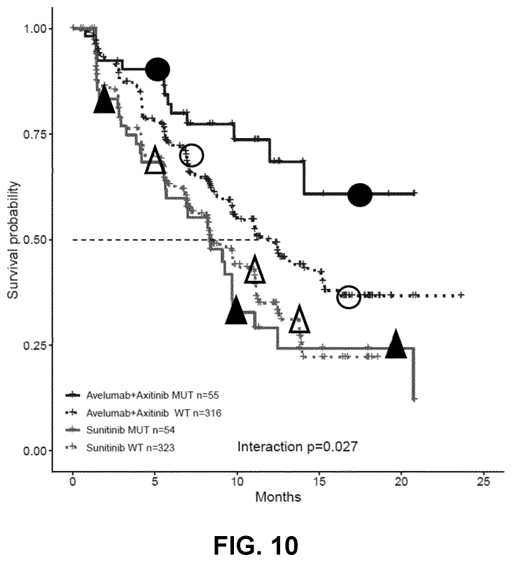

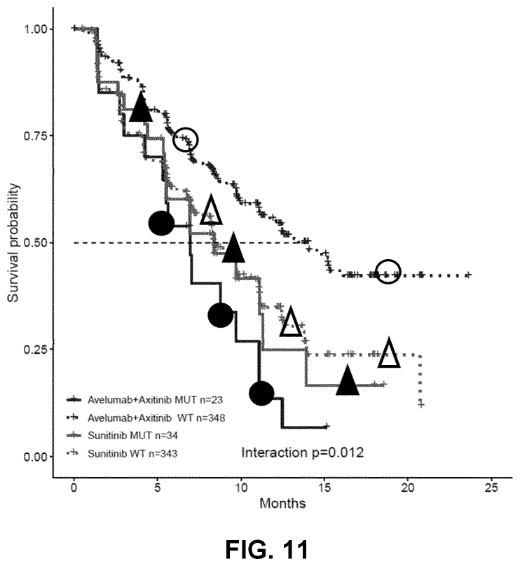

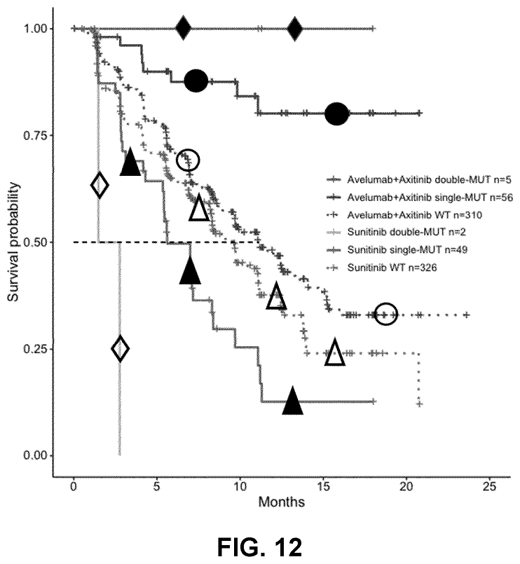

FIGS. 1-11 show graphs depicting survival probability over time (Y-axis: survival probability; X-axis months), for patients on either the avelumab+axitinib or sunitinib treatment arm and having either wild-type or mutant versions the genes CD163L1 (FIG. 1); DNMT1 (FIG. 2); MC1R (FIG. 3); ABCA1 (FIG. 4); FOXO1 (FIG. 5); IL16 (FIG. 6); MYH7B (FIG. 7); STAB2 (FIG. 8); LOC728763 (FIG. 9); SPATA31C2 (FIG. 10); or PTEN (FIG. 11). FIG. 12 shows a graph depicting survival probability over time (Y-axis: survival probability; X-axis months), for patients on either the avelumab+axitinib or sunitinib treatment arm and having mutations of zero, one or two of the genes CD163L1, DNMT1 and MC1R.

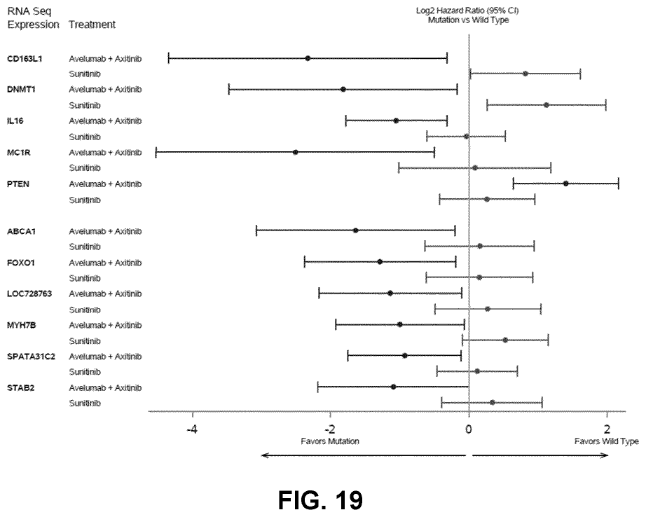

FIG. 19 shows the Log2 Hazard ratio for both treatment arms for patients having somatic mutations in the tumors in the various individual genes.

It was surprisingly found out that in the avelumab plus axitinib treatment arm, the patient group whose tumor sample contains one or more mutations in at least one of the genes selected from CD163L1, DNMT1, MC1R, FOXO1, STAB2, LOC728763, MYH7B, IL16, SPATA31C2 and ABCA1, showed far better rate of progression free survival at 15 months or even 20 months, comparing to patient group whose tumor does not contain such a mutation. Even more surprising was the finding that in the avelumab plus axitinib arm, the patient group whose tumor sample contains mutations in two of the three genes CD163L1, MC1R and DNMT1, progression free survival rate at 15 months and 20 months was 100%.

Detailed examination of the WES data revealed that mutations in the IL16, SPATA31C2, and FOXO1 genes, but not LOC728763, were most strongly associated with significantly improved PFS when they were present in the germline.

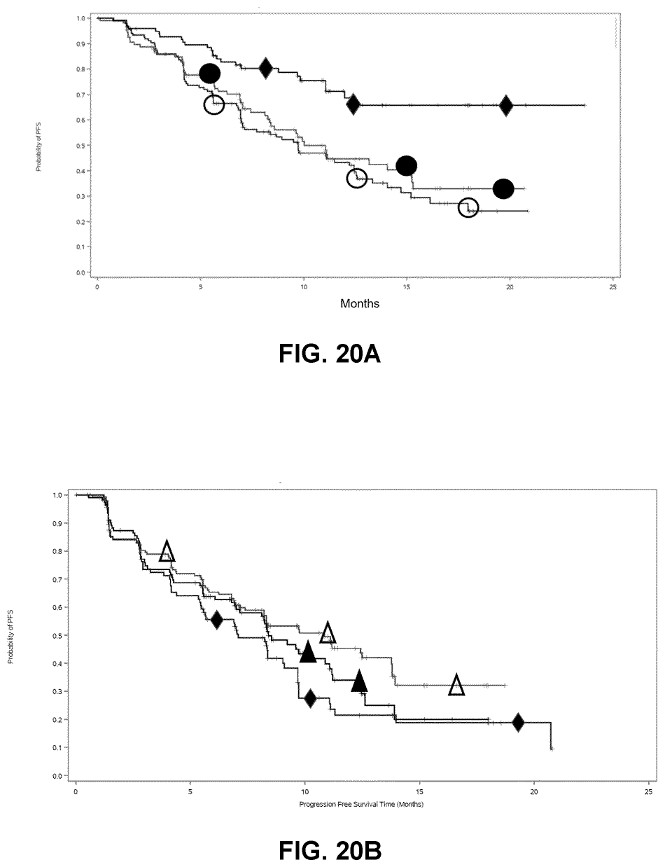

Further analysis revealed that when mutations in ≥2 genes within the list of CD163L1, DNMT1, MC1R, FOXO1, STAB2, LOC728763, MYH7B, IL16, SPATA31C2 and ABCA1 were present within a single patient, PFS was enhanced relative to wild-type patients or further enhanced relative to patients with single mutations treated with the combination of avelumab+axitinib (FIG. 20A). However, when mutations in PTEN were also present, PFS was similar to wild type. The enhancement in PFS in patients having mutations in genes within the list of CD163L1, DNMT1, MC1R, FOXO1, STAB2, LOC728763, MYH7B, IL16, SPATA31C2 and ABCA1 was not observed in the sunitinib arm (FIG. 20B).

Further analysis identified additional genes in which the presence of mutations were associated with reduced PFS in the combination of avelumab+axitinib treatment arm. Specifically, patients on the combination treatment arm that had a mutation in one or more of the following genes had shorter median PFS than patients on the combination treatment arm that did not have a mutation in: ANK2, CAPN8, CBX4, CNTRL, CYP2W1, DMRTA1, EPHA2, GREB1, HBS1L, LAMA1, LOC728392, LYST, MYOM2, NOS3, PALM3, PLKS, PTPN13, RTL1, SCAP, SHROOM2, SLCO2B1, TBX2, TENM3, TNRC6A, TTC28, USP42, ZC3H3, EFCAB6, MAP3K6, and PTPDC1. PTEN (described above) was also identified in this analysis.

### Example 4

### Immunohistochemistry (IHC) Study and Results of Patients in the Phase 3 Study of Avelumab with Axitinib Versus Sunitinib in Advanced Renal Cell Carcinoma

This example illustrates IHC study and results from the clinical trial study described in Example 2 above.

Tumor samples were collected from patients in Example 2 and were subject to immunohistochemistry (IHC), to evaluate whether there was a relationship between CD8+ cells infiltrating the tumor and clinical outcome. CD8 expression was assessed by immunohistochemistry using clone C8/144B and reported in terms of the number of CD8+ cells in relation to the total number of CD8+ cells in the tumor area or at the invasive margin, with the median value as the cut point.

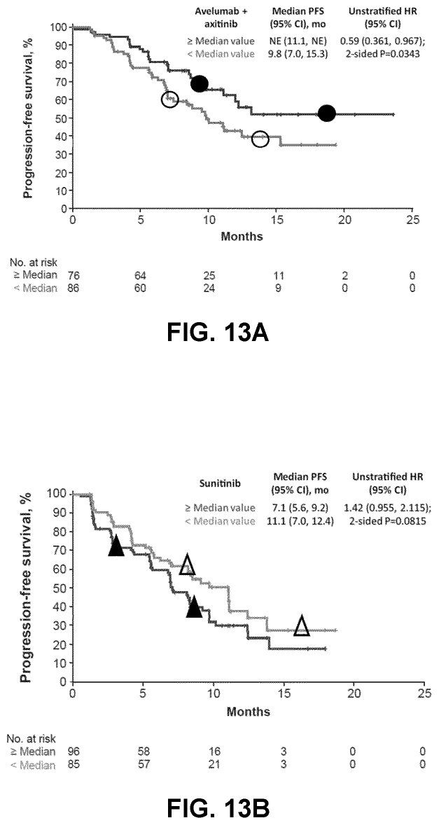

In patients with high numbers of CD8+ cells at the invasive margin, median PFS with avelumab+axitinib was not estimable [NE] (95% CI, 11.1 months, NE) vs 7.1 months (95% CI, 5.6, 9.2) with sunitinib (FIGS. 13A and 13B). In patients with low numbers of CD8+ cells, for patients on the avelumab+axitinib arm median PFS was 9.8 (95% CI, 7.0, 15.3) vs 11.1 months (95% CI, 7.0, 12.4) for patients on the sunitinib arm (FIGS. 13A and 13B). Among patients on the combination avelumab+axitinib arm, PFS was longer for those patients with high numbers of CD8+ cells at the invasive margin vs those with low numbers (HR=0.59; p=0.0343). Among patients on the sunitinib arm, patients with high numbers of CD8+ cells at the invasive margin had shorter PFS than those with low numbers (HR=1.42; p=0.815).

These results demonstrate that high numbers of CD8 positive cells at the invasive margin was associated with longer PFS among patients on the combination avelumab +axitinib arm and shorter PFS among patients on the sunitinib arm, compared to those with low numbers of CD8 positive cells in the respective arms.

### Example 5

### Gene Expression Study and Results of Patients in the Phase 3 Study of Avelumab with Axitinib Versus Sunitinib in Advanced Renal Cell Carcinoma

This example illustrates gene expression study and results from the clinical trial study described in Example 2 above.

Methods:

RNA sequencing and transcript quantification: Whole-transcriptome profiles were generated for 720 patients (350 on the avelumab+axitinib arm and 370 on the sunitinib arm) using RNA-seq (Illumina NovaSeq) on formalin-fixed paraffin-embedded (FFPE) tumor tissue. Transcript levels were quantitated by the Personalis ACE Cancer Transciptome Analysis pipeline which uses STAR version 2.4.2a-p1 to align reads to the NCBI hs37d5 annotation 105 reference genome and produces Transcripts Per Million (TPM) values for each gene. TPM values were log2 transformed for further analysis.

Co-expression network analysis. Weighted Gene Co-Expression Network Analysis (WGCNA) was used to find co-expressed modules in the 720 baseline transcriptomic samples. First, lowly expressed and invariant genes, i.e., genes that were expressed in <5% of samples or had standard deviation were removed. The soft power of 6 was chosen based on goodness of fit to a scale-free network. A signed WGCNA network was constructed in R³⁷ with mergeCutHeight set to 0.25 and reassignThreshold set to 0. After removing non-coding genes and modules with size <10, 25 modules were identified. Gene signature scores were computed from the average expression of genes in a module. Univariate Cox Proportional Hazards (Cox PH) model was used to assess the dependence of PFS on each gene signature, which was categorized into high vs low groups according to median signature score. Multivariate analysis was also carried out adjusting for age and sex. Modules were annotated through identifying top enriched gene sets via hypergeometric tests using public gene set collections including the MSigDB Hallmark, GO Biological Process and LM22.

To assess the robustness of identified gene modules, next was carried out a consensus WGCNA analysis. First, lowly expressed and invariant genes were filtered out, i.e., genes that were expressed in <5% of samples or had standard deviation ≤0.9 were removed. The soft power of 6 was chosen based on goodness of fit to a scale-free network. 720 baseline data were randomly subsampled down to 80% of samples for 1,000 times. With each subsampled dataset, we constructed a co-expression network using the WGCNA procedure as described above. An adjacency matrix was constructed by counting the frequencies two genes were clustered into the same network module. A consensus network was then constructed using the new adjacency matrix via the standard WGCNA approach. Edges were then filtered by adjacency measure of 0.8. Modules with size <10 were removed. Consensus WGCNA identified 23 modules. Hypergeometric tests were then carried out to annotate modules as described above.

JAVELIN Renal 101 Immuno Signature: Blinded to clinical outcome, co-expression network analysis using WGCNA identified an immune response module of 306 genes and expression of this 306-gene signature was associated with better PFS in the avelumab+axitinib arm but not in the sunitinib arm. To refine the 306-gene immune response module, we selected genes annotated for immune-related functionality and had univariate Cox PH p-value <0.01 in the combination arm. Together with the manually added CD8B gene, which barely misses the p-value=0.01 cutoff, a 26-gene signature was defined, that is, the JAVELIN Renal 101 Immuno Signature. Further consensus WGCNA analysis identified a consensus 414-gene immune response module which was associated with better PFS in the avelumab+axitinib arm but not in the sunitinib arm. All 26 genes in the JAVELIN Renal 101 Immuno Signature are also in the consensus 414-gene module, verifying the robustness of the signature. Validation of the 26-gene subset was carried out using an independent data set derived from patients with advanced RCC enrolled in the single-arm, phase 1 b JAVELIN Renal 100 clinical trial who received avelumab+axitinib (NCT02493751).

Summary of Results:

To systematically characterize underlying biological processes, a co-expression network analysis was carried out to identify network modules from baseline transcriptomic data. A group of 306 genes was found to be co-expressed, with the majority of these genes being upregulated in responders vs nonresponders. Further refinement of the module based primarily on the genes whose expression was most closely associated with improved PFS in the combination arm identified a 26-gene subset, herein referred to as the “JAVELIN Renal 101 Immuno Signature” (see above for details regarding the generation of this signature). The genes in the JAVELIN Renal 101 Immuno Signature are provided in Table 8 below, as well as a brief summary of the immunoregulatory functions of the genes.

TABLE 8

| Function | Genes |
|---|---|
| T-cell receptor signaling | CD3G, CD3E, CD8B, THEMIS, TRAT1, |
| | GRAP2, CD247 |
| T-cell activation, | CD2, * CD96, * PRF1, * CD6, IL7R, |
| proliferation, and | ITK, GPR18, EOMES, SIT1, NLRC3 |
| differentiation | |
| Natural killer cell-mediated | CD2, * CD96, * PRF1, * CD244, |
| cytotoxicity | KLRD1, SH2D1A |
| Chemokine | CCL5, XCL2 |
| Other immune response genes | CST7, GFI1, KCNA3, PSTPIP1 |

*Genes included in >1 functional group

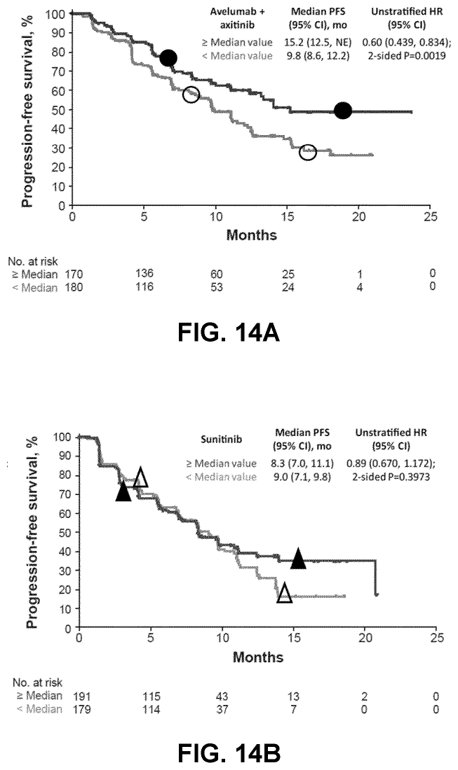

The JAVELIN Renal 101 Immuno Signature includes genes with a range of immunoregulatory functions, but displays limited overlap in composition with other published signatures, including the IMmotion 150 T_{effector} signature (McDermott D F, Huseni M A, Atkins M B, et al. Clinical activity and molecular correlates of response to atezolizumab alone or in combination with bevacizumab versus sunitinib in renal cell carcinoma. Nature Medicine. 2018;24(6):749) and the IFN-γ signature (Ayers M, Lunceford J, Nebozhyn M, et al. IFN-γ-related mRNA profile predicts clinical response to PD-1 blockade. J Clin Invest. 2017; 127(8):2930-2940). Patients with greater than median expression of the JAVELIN Renal 101 Immuno signature had longer median PFS than those with less than median expression of the signature on the avelumab+axitinib arm (15.2 vs 9.8 months; HR=0.60; p=0.0019) (FIG. 14A), but the signature did not differentiate patients on the sunitinib arm (median PFS, 8.3 vs 9.0 months; HR=0.98; p=0.3973) (FIG. 14B).

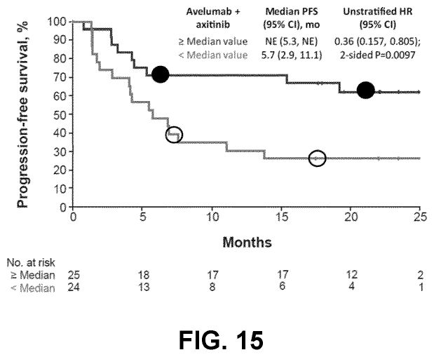

A separate, single-arm, phase 1 b JAVELIN Renal 100 clinical trial was conducted (Choueiri TK , Larkin J, Oya M, et al. Preliminary results for avelumab plus axitinib as first-line therapy in patients with advanced clear-cell renal-cell carcinoma (JAVELIN Renal 100): an open-label, dose-finding and dose-expansion, phase 1 b trial. The Lancet Oncology. 2018;19(4):451-460). A data set from this trial was analyzed, and it was determined the JAVELIN Renal 101 Immuno Signature was also enriched for responders in this trial who received the combination of avelumab+axitinib. Median PFS was NE (95% CI, 5.3 months, NE) in those with greater than median expression of the JAVELIN Renal 101 Immuno Signature and 5.7 months (95% CI, 2.9, 11.1) in those with less than median expression of the signature (HR=0.36; p=0.0097) (FIG. 15).

Other predictive or prognostic modules were identified by further analysis of the gene expression data, including a 26-gene angiogenesis gene signature, referred to herein as the “JAVELIN Renal 101 Angiogenesis Signature”. The genes in the JAVELIN Renal 101 Angiogenesis Signature are provided in Table 9 below.

TABLE 9

| | | | |
|---|---|---|---|
| NRARP | RAMP2 | ARHGEF15 | VIP |
| NRXN3 | KDR | SMAD6 | KCNAB1 |
| CALCRL | NOTCH4 | AQP1 | RAMP3 |
| TEK | FLT1 | GATA2 | CACNB2 |
| ECSCR | GJA5 | ENPP2 | CASQ2 |
| PTPRB | TBX2 | ATP1A2 | |
| CD34 | HEY2 | EDNRB | |

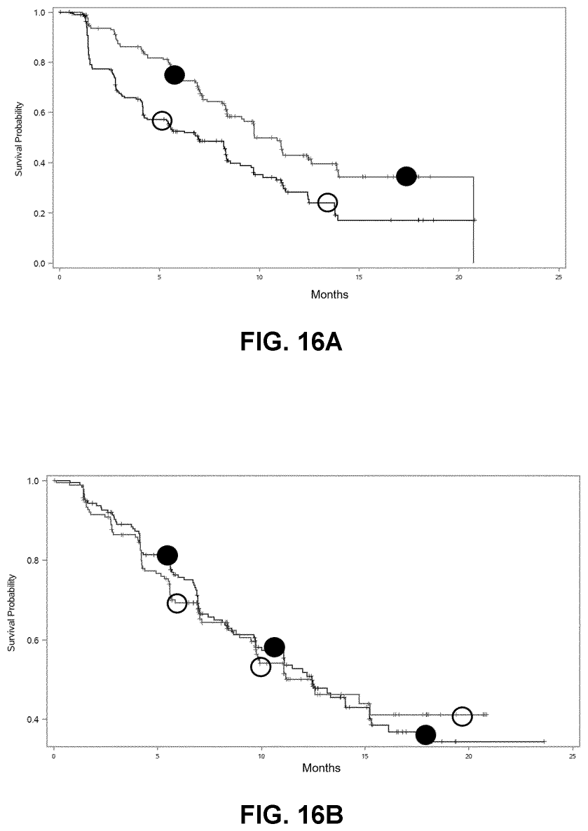

The JAVELIN Renal 101 Angiogenesis Signature significantly differentiated PFS for patients on the sunitinib arm (HR=0.56; p<0.0001) (FIG. 16A) but not the avelumab+axitinib arm (HR=0.98; p=0.8819) (FIG. 16B).

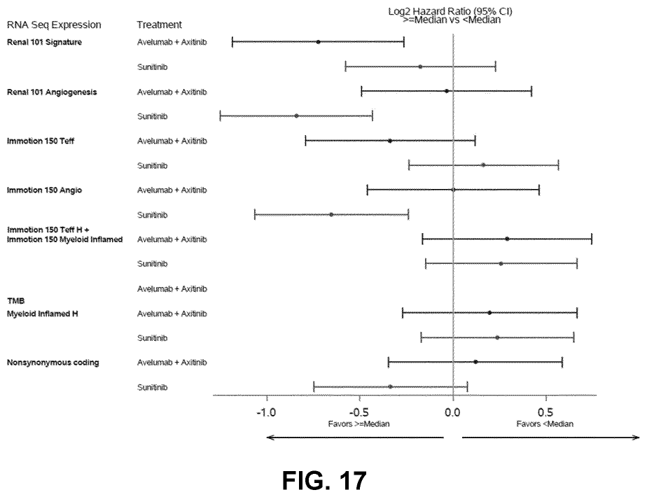

Published gene signatures were applied to PFS data from JAVELIN Renal 101 patients. Patients who received avelumab+axitinib and had high expression of the IMmotion 150 T_{effector} signature showed a trend toward longer PFS than those on the sunitinib arm (FIG. 17). Patients on the sunitinib arm with high expression of the IMmotion 150 angiogenesis signature had significantly improved PFS (HR=0.64; p=0.0018), but this signature did not differentiate PFS in patients on the avelumab+axitinib arm. Neither the IMmotion 150 myeloid inflamed, T_{effector}^{high}/Myeloid inflamed^{high} gene signatures, nor tumor mutational burden (TMB) differentiated PFS in either arm (TMB; avelumab+axitinib arm: HR, 1.09; 95% CI, 0.79, 1.50; p=0.6084; sunitinib arm: HR, 0.79; 95% CI, 0.60, 1.05; p=0.1092).

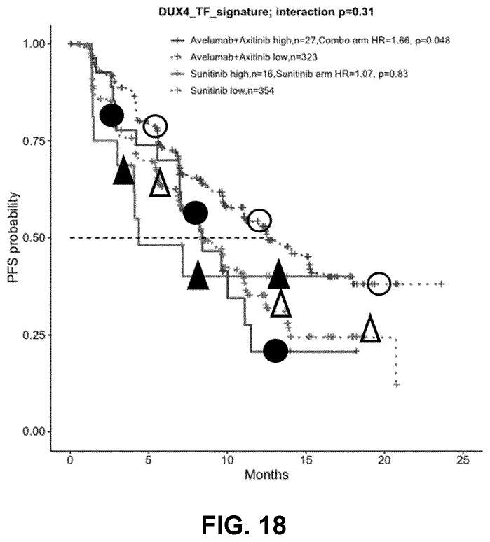

Additional analysis of the gene expression data revealed that patients having a greater than median expression of the DUX4 gene signature had a shorter PFS than those with less than median expression of the DUX4 gene signature on the avelumab+axitinib arm (FIG. 18). In addition, high expression of the DUX4 gene signature was found to be correlated with loss of HLA expression. DUX4 is a transcription factor, and DUX4 expression can be assessed by examining the expression of one or more or more genes for which DUX4 increases expression. For this study, the DUX4 gene signature examined included the genes: ZSCAN4, PRAMEF1, PRYDS, KHDC1L, MBD3L2, and TRIM43.

### Example 6

### Additional Gene Expression Study and Results of Patients in the Phase 3 Study of Avelumab with Axitinib Versus Sunitinib in Advanced Renal Cell Carcinoma

This example illustrates additional gene expression study and results from the clinical trial study described in Example 2 above.

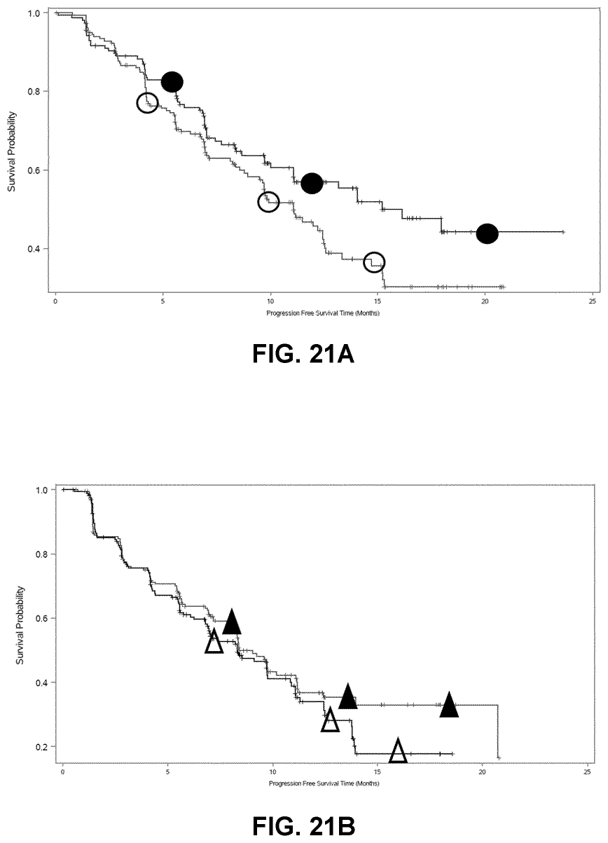

Following the identification as described in Example 3 above of genetic mutations in one or more of CD163L1, DNMT1, MC1R, FOXO1, STAB2, LOC728763, MYH7B, IL16, SPATA31C2 and ABCA1 genes as being associated with profoundly different PFS, the gene expression data from the trial population was re-examined to explore alterations to biological processes that may underly these observations. Patients who harbored of any of the noted mutations (n=101) were grouped together, and their gene expression profiles were compared to the remaining population (n=514). Strikingly, expression of a single gene, UTS2 (a potent vasoconstrictor also associated with an inflammatory response), stood out as highly differentially expressed between these two groups (FIGS. 21A and 21B). Specifically, in the avelumab+axitinib treatment arm (FIG. 21A), for patients having greater than median expression of the UTS2 gene, median PFS was 16.1 months, whereas for patients having less than median expression of the UTS2 gene, median PFS was 11.1 months. This difference in PFS was not observed in the sunitinib arm (FIG. 21B), where UTS2 gene expression level did not affect PFS.

Most of the tumor samples had very low expression of the UTS2 gene (below 0.02 log2 Transcripts Per Million (TPM)). Tumor samples having a greater than median expression of UTS2 had greater than 0.02 log2 TPM.

Single gene RNAseq data indicated that, irrespective of status of mutations in one or more of CD163L1, DNMT1, MC1R, FOXO1, STAB2, LOC728763, MYH7B, IL16, SPATA31C2 and ABCA1 genes, patients whose tumors express higher levels of UTS2 have extended PFS relative to those with lower expression, when treated with avelumab and axitinib.

In addition, information in public databases (e.g. The Cancer Genome Atlas Program) indicates that mutations in CD163L1, DNMT1, MC1R, FOXO1, STAB2, LOC728763, MYH7B, IL16, SPATA31C2 and ABCA1 genes are present in ˜5-25% patients across a range of tumor types.

### Example 7

### Analysis of HLA Alleles in Patients in the Phase 3 Study of Avelumab with Axitinib Versus Sunitinib in Advanced Renal Cell Carcinoma

This example illustrates analysis of HLA alleles in patients from the clinical trial study described in Example 2 above.

Distribution of HLA types within the trial population in the study described above was examined. Of the alleles present in 5% of more of the patients, 5 HLA alleles were significantly associated with differences in PFS relative to other HLA types. These included: A*01:01, A*03:01, B*40:02, B*57:01 and C*06:02. Patients having HLA types A*01:01 and B*57:01 had increased PFS in the combination arm, whereas patients having HLA type A*03:01 had decreased PFS in the combination arm. Patients having HLA type B*40:02 had decreased PFS on the sunitinib arm with no difference in combination arm. Patients having HLA type C*06:02 had increased PFS on the sunitinib arm and a trend towards increased PFS on the combination arm.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A method for treating cancer in a patient, wherein the cancer in the patient is pre-determined
(a) to contain one or more protein altering mutations in one or more gene(s) selected from the group consisting of CD163L1, DNMT1, MC1R, FOXO1, STAB2, LOC728763, MYH7B, IL16, SPATA31C2, ARVCF, and ABCA1, and/or
(b) to not contain a protein altering mutation in one or more gene(s) selected from the group consisting of the PTEN, ANK2, CAPN8, CBX4, CNTRL, CYP2W1, DMRTA1, EPHA2, GREB1, HBS1L, LAMA1, LOC728392, LYST, MYOM2, NOS3, PALMS, PLK5, PTPN13, RTL1, SCAP, SHROOM2, SLCO2B1, TBX2, TENM3, TNRC6A, TTC28, USP42, ZC3H3, EFCAB6, MAP3K6, and PTPDC1;
comprising administering to the patient a therapeutically effective amount of a PD-1 axis binding antagonist.

2. The method of claim 1, further comprising administering to the patient a therapeutically effective amount of a VEGF pathway inhibitor.

3. The method of claim 2, wherein the VEGF pathway inhibitor is a VEGFR inhibitor.

4. The method of claim 2, wherein the VEGF pathway inhibitor is axitinib or a pharmaceutically acceptable salt thereof.

5. The method of claim 1, wherein the PD-1 axis binding antagonist is an anti-PD-1 antibody.

6. The method of claim 1, wherein the PD-1 axis binding antagonist is an anti-PD-L1 antibody.

7. The method of claim 6, wherein the anti-PD-L1 antibody is selected from the group consisting of avelumab, atezolizumab and durvalumab.

8. The method of claim 1, wherein the PD-1 axis binding antagonist is administered at a dose of about 5 mg/kg, about 10 mg/kg, about 200 mg, about 240 mg, about 400 mg, about 800 mg or about 1200 mg, and is administered about once a week, or about once every two, three, four, five weeks or six weeks; and the VEGF pathway inhibitor is administered at a dose of about 3 mg/kg, about 5 mg/kg, about 3 mg, or about 5 mg and is administered twice daily.

9. The method of claim 1 wherein the PD-1 axis binding antagonist is avelumab and administered at a dose of about 800 mg once every two weeks, and the VEGF pathway inhibitor is axitinib and administered at a dose of about 5 mg twice daily.

10. A medicament comprising a PD-1 axis binding antagonist for use in treating a cancer in a patient, wherein the cancer of the patient is pre-determined as
(a) containing one or more protein altering mutations in one or more gene(s) selected from the group consisting of CD163L1, DNMT1, MC1R, FOXO1, STAB2, LOC728763, MYH7B, IL16, SPATA31C2, ARVCF, and ABCA1, and/or
(b) not containing a protein altering mutation in one or more gene(s) selected from the group consisting of PTEN, ANK2, CAPN8, CBX4, CNTRL, CYP2W1, DMRTA1, EPHA2, GREB1, HBS1L, LAMA1, LOC728392, LYST, MYOM2, NOS3, PALMS, PLKS, PTPN13, RTL1, SCAP, SHROOM2, SLCO2B1, TBX2, TENM3, TNRC6A, TTC28, USP42, ZC3H3, EFCAB6, MAP3K6, and PTPDC1.

11. The medicament of claim 10, wherein the medicament is to be used in combination with a VEGF pathway inhibitor.

12-14. (canceled)

15. A method of treating a patient having a cancer, comprising administering to the patient a therapeutically effective amount of a PD-1 axis binding antagonist, wherein the expression level of the gene UTS2 in a sample obtained from the patient has been determined to be increased as compared to a reference level.

16. A method of treating a patient having a cancer, comprising administering to the patient a therapeutically effective amount of a PD-1 axis binding antagonist, wherein the expression level of at least one gene selected from the group consisting of CD3G, CD3E, CD8B, THEMIS, TRAT1, GRAP2, CD247, CD2, CD96, PRF1, CD6, IL7R, ITK, GPR18, EOMES, SIT1, NLRC3, CD244, KLRD1, SH2D1A, CCL5, XCL2, CST7, GFI1, KCNA3, PSTPIP1 in a sample obtained from the patient has been determined to be increased as compared to a reference level.

17-19. (canceled)

20. The method claim 1, wherein the cancer is bladder cancer, breast cancer, clear cell kidney cancer, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), liver cancer, mantle cell lymphoma (MCL), multiple myeloma (MM), myelodysplastic syndrome (MDS), non-Hodgkin's lymphoma (NHL), Squamous Cell Carcinoma of the Head and Neck (SCCHN), small lymphocytic lymphoma (SLL), endometrial cancer, B-cell acute lymphoblastic leukemia, colorectal cancer, glioblastoma, cervical cancer, penile cancer, or non-melanoma skin cancer.

21. The method of claim 20, wherein the cancer is renal cell carcinoma.


SEQUENCE LISTING (36 sequences)

SEQ ID NO: 1 - protein, 444 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 443 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 221 aa, Artificial Sequence
  Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 113 aa, Artificial Sequence
  Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 5 - protein, 117 aa, Artificial Sequence
  Synthetic Construct
QVQLVQSGAEVKKPGASVKVSCKASGYTFTSYWINWVRQAPGQGLEWMGNIWPGSSLTNYNEKFKNRVTMTRDTSTSTVYMELSSLRSEDTAVYYCARLLTGTFAYWGQGTLVTVSS

SEQ ID NO: 6 - protein, 113 aa, Artificial Sequence; identical to SEQ ID NO: 4
  Synthetic Construct

SEQ ID NO: 7 - protein, 432 aa, Homo sapiens
QVQLVESGGGWQPGRSLRLDCKASGITFSNSGMHWVRQAPGKGLEWVAVRWYDGSKRYYADSVKGRFTISRDNSKNTLFLQMNSLRAEDTAVYYCATNDDYWGQGTLVTVSSASTKGPSVFPLAPCSRSTSESTAALGCLVDYFPEPVTVSWNSGALTSGVHTFPAVLQSSGLYSLSSVVTVPSSSLGTTYTCNVDHKPSNTKVDRVESYGPPCPPCPAPEFLGGPSVFLFPPKPKDTLMISRTPEVTCWVDVSQEDPEVQFNWYYDGVEVHNATKPREEQFNSTYRVVSVLTVLHQDWLNGKEYKCKVSNKGLPSSIEKTISKAGQPREPQVYTLPPSQEEMTKNQVSLTCLVKGFYPSDIAVEWESNGQPEKNYKTTPPVLDSDGSFFLYSRLTVDKSRWQEGNVFSCSVMHEALHNHYTQKSLSLSLGK

SEQ ID NO: 8 - protein, 208 aa, Homo sapiens
EIVLTQSPATLSLSPGERATLSCRASQSVSSYLAWYQQPGQAPRLLIYDASNRATGIPARFSGSGSGTDFTLTISSLEPEDFAVYYCQQSSNWPRTFGQGTKVEIRTVAAPSVFIFPPSDEQLSGTASVVCLLNNFYPREAVQWKVDNALQSGNSQESVTEQDSDSTYSLSSTLTLSKADYEKHKVYACEVTHQGLSSPVTSFNRGEC

SEQ ID NO: 9 - protein, 447 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 432 aa, Artificial Sequence
  Synthetic Construct
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 118 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 13 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 14 - protein, 117 aa, Artificial Sequence
  Synthetic Construct
EVQLLESGGGLVQPGGSLRLSCAASGFTFSSYIMWVRQAPGKGLEWVSSIYPSGGITFYADKGRFTISRDNSKNTLYLQMNSLRAEDTAVYYCARIKLGTVTTVDYWGQGTLVTVSS

SEQ ID NO: 15 - protein, 110 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 16 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 17 - protein, 5 aa, Artificial Sequence
  Synthetic Construct
SYIMM

SEQ ID NO: 18 - protein, 11 aa, Artificial Sequence
  Synthetic Construct
SIYPSGGITFY

SEQ ID NO: 19 - protein, 11 aa, Artificial Sequence
  Synthetic Construct
IKLGTVTTVDY

SEQ ID NO: 20 - protein, 14 aa, Artificial Sequence
  Synthetic Construct
TGTSSDVGGYNYVS

SEQ ID NO: 21 - protein, 7 aa, Artificial Sequence
  Synthetic Construct
DVSNRPS

SEQ ID NO: 22 - protein, 10 aa, Artificial Sequence
  Synthetic Construct
SSYTSSSTRV

SEQ ID NO: 23 - protein, 450 aa, Artificial Sequence
  Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 216 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 25 - protein, 1453 aa, Homo sapiens
MMLPQNSWHIDFGRCCCHQNLFSAVVTCILLLNSCFLISSFNGTDLELRLVNGDGPCSGTVEVKFQGQWGTVCDDGWNTTASTVVCKQLGCPFSFAMFRFGQAVTRHGKIWLDDVSCYGNESALWECQHREWGSHNCYHGEDVGVNCYGEANLGLRLVDGNNSCSGRVEVKFQERWGTICDDGWNLNTAAVVCRQLGCPSSFISSGVVNSPAVLRPIWLDDILCQGNELALWNCRHRGWGNHDCSHNEDVTLTCYDSSDLELRLVGGTNRCMGRVELKIQGRWGTVCHHKWNNAAADVVCKQLGCGTALHFAGLPHLQSGSDVVWLDGVSCSGNESFLWDCRHSGTVNFDCLHQNDVSVICSDGADLELRLADGSNNCSGRVEVRIHEQWWTICDQNWKNEQALVVCKQLGCPFSVFGSRRAKPSNEARDIWINSISCTGNESALWDCTYDGKAKRTCFRRSDAGVICSDKADLDLRLVGAHSPCYGRLEVKYQGEWGTVCHDRWSTRNAAVVCKQLGCGKPLHVFGMTYFKEASGPIWLDDVSCIGNESNIWDCEHSGWGKHNCVHREDVIVTCSGDATWGLRLVGGSNRCSGRLEVYFQGRWGTVCDDGWNSKAAAVVCSQLDCPSSIIGMGLGNASTGYGKIWLDDVSCDGDESDLWSCRNSGWGNNDCSHSEDVGVICSDASDMELRLVGGSSRCAGKVEVNVQGAVGILCANGWGMNIAEVVCRQLECGSAIRVSREPHFTERTLHILMSNSGCTGGEASLWDCIRWEWKQTACHLNMEASLICSAHRQPRLVGADMPCSGRVEVKHADTWRSVCDSDFSLHAANVLCRELNCGDAISLSVGDHFGKGNGLTWAEKFQCEGSETHLALCPIVQHPEDTCIHSREVGVVCSRYTDVRLVNGKSQCDGQVEINVLGHWGSLCDTHWDPEDARVLCRQLSCGTALSTTGGKYIGERSVRVWGHRFHCLGNESLLDNCQMTVLGAPPCIHGNTVSVICTGSLTQPLFPCLANVSDPYLSAVPEGSALICLEDKRLRLVDGDSRCAGRVEIYHDGFWGTICDDGWDLSDAHVVCQKLGCGVAFNATVSAHFGEGSGPIWLDDLNCTGMESHLWQCPSRGWGQHDCRHKEDAGVICSEFTALRLYSETETESCAGRLEVFYNGTWGSVGRRNITTAIAGIVCRQLGCGENGVVSLAPLSKTGSGFMWVDDIQCPKTHISIWQCLSAPWERRISSPAEETWITCEDRIRVRGGDTECSGRVEIWHAGSWGTVCDDSWDLAEAEVVCQQLGCGSALAALRDASFGQGTGTIWLDDMRCKGNESFLWDCHAKPWGQSDCGHKEDAGVRCSGQSLKSLNASSGHLALILSSIFGLLLLVLFILFLTWCRVQKQKHLPLRVSTRRRGSLEENLFHEMETCLKREDPHGTRTSDDTPNHGCEDASDTSLLGVLPASEATK

SEQ ID NO: 26 - protein, 1632 aa, Homo sapiens
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKRSRDPPASASQVTGIRAEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPDLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQVYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 27 - protein, 317 aa, Homo sapiens
MAVQGSQRRLLGSLNSTPTAIPQLGLAANQTGARCLEVSISDGLFLSLGLVSLVENALVVATIAKNRNLHSPMYCFICCLALSDLLVSGSNVLETAVILLLEAGALVARAAVLQQLDNVIDVITCSSMLSSLCFLGAIAVDRYISIFYALRYHSIVTLPRARRAVAAIWVASVVFSTLFIAYYDHVAVLLCLVVFFLAMLVLMAVLYVHMLARACQHAQGIARLHKRQRPVHQGFGLKGAVTLTILLGIFFLCWGPFFLHLTLIVLCPEHPTCGCIFKNFNLFLALIICNAIIDPLIYAFHSQELRRTLKEVLTCSW

SEQ ID NO: 28 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 29 - protein, 2551 aa, Homo sapiens
MMLQHLVIFCLGLVVQNFCSPAETTGQARRCDRKSLLTIRTECRSCALNLGVKCPDGYTMITSGSVGVRDCRYTFEVRTYSLSLPGCRHICRKDYLQPRCCPGRWGPDCIECPGGAGSPCNGRGSCAEGMEGNGTCSCQEGFGGTACETCADDNLFGPSCSSVCNCVHGVCNSGLDGDGTCECYSAYTGPKCDKPIPECAALLCPENSRCSPSTEDENKLECKCLPNYRGDGKYCDPINPCLRKICHPHAHCTYLGPNRHSCTCQEGYRGDGQVCLPVDPCQINFGNCPTKSTVCKYDGPGQSHCECKEHYQNFVPGVGCSMTDICKSDNPCHRNANCTTVAPGRTECICQKGYVGDGLTCYGNIMERLRELNTEPRGKWQGRLTSFISLLDKAYAWPLSKLGPFTVLLPTDKGLKGFNVNELLVDNKAAQYFVKLHIIAGQMNIEYMNNTDMFYTLTGKSGEIFNSDKDNQIKLKLHGGKKKVKIIQGDIIASNGLLHILDRAMDKLEPTFESNNEQTIMTMLQPRYSKFRSLLEETNLGHALDEDGVGGPYTIFVPNNEALNNMKDGTLDYLLSPEGSRKLLELVRYHIVPFTQLEVATLISTPHIRSMANQLIQFNTTDNGQILANDVAMEEIEITAKNGRIYTLTGVLIPPSIVPILPHRCDETKREMKLGTCVSCSLVYWSRCPANSEPTALFTHRCVYSGRFGSLKSGCARYCNATVKIPKCCKGFYGPDCNQCPGGFSNPCSGNGQCADSLGGNGTCICEEGFQGSQCQFCSDPNKYGPRCNKKCLCVHGTCNNRIDSDGACLTGTCRDGSAGRLCDKQTSACGPYVQFCHIHATCEYSNGTASCICKAGYEGDGTLCSEMDPCTGLTPGGCSRNAECIKTGTGTHTCVCQQGWTGNGRDCSEINNCLLPSAGGCHDNASCLYVGPGQNECECKKGFRGNGIDCEPITSCLEQTGKCHPLASCQSTSSGVWSCVCQEGYEGDGFLCYGNAAVELSFLSEAAIFNRWINNASLQPTLSATSNLTVLVPSQQATEDMDQDEKSFWLSQSNIPALIKYHMLLGTYRVADLQTLSSSDMLATSLQGNFLHLAKVDGNITIEGASIVDGDNAATNGVIHIINKVLVPQRRLTGSLPNLLMRLEQMPDYSIFRGYIIQYNLANAIEAADAYTVFAPNNNAIENYIREKKVLSLEEDVLRYHVVLEEKLLKNDLHNGMHRETMLGFSYFLSFFLHNDQLYVNEAPINYTNVATDKGVIHGLGKVLEIQKNRCDNNDTTIIRGRCRTCSSELTCPFGTKSLGNEKRRCIYTSYFMGRRTLFIGCQPKCVRTVITRECCAGFFGPQCQPCPGNAQNVCFGNGICLDGVNGTGVCECGEGFSGTACETCTEGKYGIHCDQACSCVHGRCNQGPLGDGSCDCDVGWRGVHCDNATTEDNCNGTCHTSANCLTNSDGTASCKCAAGFQGNGTICTAINACEISNGGCSAKADCKRTTPGRRVCTCKAGYTGDGIVCLEINPCLENHGGCDKNAECTQTGPNQAACNCLPAYTGDGKVCTLINVCLTKNGGCSEFAICNHTGQVERTCTCKPNYIGDGFTCRGSIYQELPKNPKTSQYFFQLQEHFVKDLVGPGPFTVFAPLSAAFDEEARVKDWDKYGLMPQVLRYHVVACHQLLLENLKLISNATSLQGEPIVISVSQSTVYINNKAKIISSDIISTNGIVHIIDKLLSPKNLLITPKDNSGRILQNLTTLATNNGYIKFSNLIQDSGLLSVITDPIHTPVTLFWPTDQALHALPAEQQDFLFNQDNKDKLKEYLKFHVIRDAKVLAVDLPTSTAWKTLQGSELSVKCGAGRDIGDLFLNGQTCRIVQRELLFDLGVAYGIDCLLIDPTLGGRCDTFTTFDASGECGSCVNTPSCPRWSKPKGVKQKCLYNLPFKRNLEGCRERCSLVIQIPRCCKGYFGRDCQACPGGPDAPCNNRGVCLDQYSATGECKCNTGFNGTACEMCWPGRFGPDCLPCGCSDHGQCDDGITGSGQCLCETGWTGPSCDTQAVLPAVCTPPCSAHATCKENNTCECNLDYEGDGITCTVVDFCKQDNGGCAKVARCSQKGTKVSCSCQKGYKGDGHSCTEIDPCADGLNGGCHEHATCKMTGPGKHKCECKSHYVGDGLNCEPEQLPIDRCLQDNGQCHADAKCVDLHFQDTTVGVFHLRSPLGQYKLTFDKAREACANEAATMATYNQLSYAQKAKYHLCSAGWLETGRVAYPTAFASQNCGSGVVGIVDYGPRPNKSEMWDVFCYRMKDVNCTCKVGYVGDGFSCSGNLLQVLMSFPSLTNFLTEVLAYSNSSARGRAFLEHLTDLSIRGTLFVPQNSGLGENETLSGRDIEHHLANVSMFFYNDLVNGTTLQTRLGSKLLITASQDPLQPTETRFVDGRAILQWDIFASNGIIHVISRPLKAPPAPVTLTHTGLGAGIFFAIILVTGAVALAAYSYFRINRRTIGFQHFESEEDINVAALGKQQPENISNPLYESTTSAPPEPSYDPFTDSEERQLEGNDPLRTL

SEQ ID NO: 30 - protein, 1061 aa, Homo sapiens
MRAAIERRWRREQELCLQLKSSQALVASLQEQLSESRRELWAAQKLQQERAREQAREREALRGQLEAQRLEVQQCRASCKLLGREKAALEMVVEELKGKADAADAEKQGLEAEAAELQRSLLLQAERREELALRRERSCRALETSQGRLQQLEEKVSGLREELASVREALSTAQLQRDVVESEREGLRSALARAECSNADLELLVRRLKSEGVEQRDSLAAMAALMEGLAQDKSALNHLALQLEQERDQLREQRKTLEQERARAGEQLAQAEQQLALERAERRGLQQACGRLEQRQEQLEGQAALLGREKAQLQEQVGQVTCQKQALEEQLAQSLQDQEAQMGTLQQALQGKDALSEERAQLLAKQEALERQGRLAAEEAADLRVERDSLESSLLEAQQLATKLQEQLEEEARSAGLARQALQGAPRALPQLLPRKSAMEGPLEWSSVPTLEMEQLQSDWEVQEMKLRQDTVRLQRQVAQQEREAQRALESQALAHREALAQLQREKETLSLTLAEEKEVARCQLEQEKELVTKSAAEREALKGEIQSLKQERDESLLQLEHKMQQVMALSLKETERSLLSEELSRARRTLERVQQEAQSQQEQAQRGLLWSQCWLLEPSLELALSLPRTRYQACQPAGLPGELVKGSFATSAQKMEIKTVERSGRRGMPAKEHQAPDDRTANTKPALEGAIPHQPPPASRLLLPGGLATISATTEELKALQAQFEDAITAHQRETTALRESLQDLAAERGDVEREAERLRAQLTVAQEGLAALRQELQGVEESREGLHREAQEARRALSDEAREKDVLLLFNSELRATICRAEQEKASFKRSKEEKEQKLLILEEAQAALQQEASALRAHLWELEQAGGDARQELRELHRQVRTLKAENQRRSGEAHELQAQCSQEVLELRRQAAKAEAKHEGARKEVLGLQRKLAEVEAAGEAHGQRLQEHLRESRGAEQTLRAELHSVTRKLQEASGVADALQARLDQACHRIHSLEQELAQAEGARQDAEAQLGRLCSTLRRGLGLQRQSPWASPEQPGSPTKGLTTHTVRDSDRQL

SEQ ID NO: 31 - protein, 1983 aa, Homo sapiens
MSGNKRGSRASCPHRGAECLLPWAALNLQGFQLLLLHPSATAMMDVSELGESARYLRQGYQEMTKVHTIPWDGKKRVWVPDEQDAYVEAEVKSEATGGRVTVETKDQKVLMVREAELQPMNPPRFDLLEDMAMMTHLNEASVLHNLRQRYARWMIYTYSGLFCVTINPYKWLPVYTASVVAAYKGKRRSDSPPHIYAVADNAYNDMLRNRDNQSMLITGESGAGKTVNTKRVIQYFAIVAALGDGPGKKAQFLATKTGGTLEDQIIEANPAMEAFGNAKTLRNDNSSRFGKFIRIHFGPSGKLASADIDSYLLEKSRVIFQLPGERSYHVYYQILSGRKPELQDMLLLSMNPYDYHFCSQGVITVDNMNDGEELIATDHAMDILGFSVDEKCACYKIVGALLHFGNMKFKQKQREEQAEADGTESADKAAYLMGVSSGDLLKGLLHPRVRVGNEYVTKGQSVEQVVFAVGALAKATYDRLFRWLVSRINQTLDTKLPRQFFIGVLDIAGFEIFEFNSFEQLCINFTNEKLQQFFNQHMFVLEQEEYKREGIDWVFIDFGLDLQPCIDLIEKPLGILSILEEECMFPKASDASFRAKLYDNHAGKSPNFQQPRPDKKRKYQAHFEVVHYAGVVPYSIVGWLEKNKDPLNETVVPIFQKSQNRLLATLYENYAGSCSTEPPKSGVKEKRKKAASFQTVSQLHKENLNKLMTNLRATQPHFVRCIVPNENKTPGVMDAFLVLHQLRCNGVLEGIRICRQGFPNRLLYTDFRQRYRILNPSAIPDDTFMDSRKATEKLLGSLDLDHTQYQFGHTKVFFKAGLLGVLEELRDQRLAKVLTLLQARSRGRLMRLEYQRLLGGRDALFTIQWNIRAFNAVKNWSWMKLFFKMKPLLRSAQAEEELAALRAELRGLRGALAAAEAKRQELEETHVSITQEKNDLALQLQAEQDNLADAEERCHLLIKSKVQLEGKVKELSERLEDEEEVNADLAARRRKLEDECTELKKDIDDLELTLAKAEKEKQATENKVKNLTEEMAALDESVARLTKEKKALQEAHQQALGDLQAEEDRVSALTKAKLRLEQQVEDLECSLEQEKKLRMDTERAKRKLEGDLKLTQESVADAAQDKQQLEEKLKKKDSELSQLSLRVEDEQLLGAQMQKKIKELQARAEELEEELEAERAARARVEKQRAEAARELEELSERLEEAGGASAGQREGCRKREAELGRLRRELEEAALRHEATVAALRRKQAEGAAELGEQVDSLQRVRQKLEKEKSELRMEVDDLAANVETLTRAKASAEKLCRTYEDQLSEAKIKVEELQRQLADASTQRGRLQTESGELSRLLEEKECLISQLSRGKALAAQSLEELRRQLEEESKAKSALAHAVQALRHDCDLLREQHEEEAEAQAELQRLLSKANAEVAQWRSKYEADAIQRTEELEEAKKKLALRLQEAEEGVEAANAKCSSLEKAKLRLQTESEDVTLELERATSAAAALDKKQRHLERALEERRRQEEEMQRELEAAQRESRGLGTELFRLRHGHEEALEALETLKRENKNLQEEISDLTDQVSLSGKSIQELEKTKKALEGEKSEIQAALEEAEGALELEETKTLRIQLELSQVKAEVDRKLAEKDEECANLRRNHQRAVESLQASLDAETRARNEALRLKKKMEGDLNDLELQLGHATRQATEAQAATRLMQAQLKEEQAGRDEEQRLAAELHEQAQALERRASLLAAELEELRAALEQGERSRRLAEQELLEATERLNLLHSQNTGLLNQKKKLEADLAQLSGEVEEAAQERREAEEKAKKAITDAAMMAEELKKEQDTSAHLERMKKTLEQTVRELQARLEEAEQAALRGGKKQVQKLEAKVRELEAELDAEQKKHAEALKGVRKHERRVKELAYQAEEDRKNLARMQDLVDKLQSKVKSYKRQFEEAEQQANTNLAKYRKAQHELDDAEERADMAETQANKLRARTRDALGPKHKE

SEQ ID NO: 32 - protein, 1331 aa, Homo sapiens
MESHSRAGKSRKSAKFRSISRSLMLCNAKTSDDGSSPDEKYPDPFEISLAQGKEGIFHSSVQLADTSEAGPSSVPDLALASEAAQLQAAGNDRGKTCRRIFFMKESSTASSREKPGKLEAQSSNFLFPKACHQRARSNSTSVNPYCTREIDFPMTKKSAAPTDRQPYSLCSNRKSLSQQLDCPAGKAAGTSRPTRSLSTAQLVQPSGGLQASVISNIVLMKGQAKGLGFSIVGGKDSIYGPIGIYVKTIFAGGAAAADGRLQEGDEILELNGESMAGLTHQDALQKFKQAKKGLLTLTVRTRLTAPPSLCSHLSPPLCRSLSSSTCITKDSSSFALESPSAPISTAKPNYRIMVEVSLQKEAGVGLGIGLCSVPYFQCISGIFVHTLSPGSVAHLDGRLRCGDEIVEISDSPVHCLTLNEVYTILSHCDPGPVPIIVSRHPDPQVSEQQLKEAVAQAVENTKFGKERHQWSLEGVKRLESSWHGRPTLEKEREKNSAPPHRRAQKVMIRSSSDSSYMSGSPGGSPGSGSAEKPSSDVDISTHSPSLPLAREPVVLSIASSRLPQESPPLPESRDSHPPLRLKKSFEILVRKPMSSKPKPPPRKYFKSDSDPQKSLEERENSSCSSGHTPPTCGQEARELLPLLLPQEDTAGRSPSASAGCPGPGIGPQTKSSTEGEPGWRRASPVTQTSPIKHPLLKRQARMDYSFDTTAEDPWVRISDCIKNLFSPIMSENHGHMPLQPNASLNEEEGTQGHPDGTPPKLDTANGTPKVYKSADSSTVKKGPPVAPKPAWFRQSLKGLRNRASDPRGLPDPALSTQPAPASREHLGSHIRASSSSSSIRQRISSFETFGSSQLPDKGAQRLSLQPSSGEAAKPLGKHEEGRFSGLLGRGAAPTLVPQQPEQVLSSGSPAASEARDPGVSESPPPGRQPNQKTLPPGPDPLLRLLSTQAEESQGPVLKMPSQRARSFPLTRSQSCETKLLDEKTSKLYSISSQVSSAVMKSLLCLPSSISCAQTPCIPKEGASPTSSSNEDSAANGSAETSALDTGFSLNLSELREYTEGLTEAKEDDDGDHSSLQSGQSVISLLSSEELKKLIEEVKVLDEATLKQLDGIHVTILHKEEGAGLGFSLAGGADLENKVITVHRVFPNGLASQEGTIQKGNEVLSINGKSLKGTTHHDALAILRQAREPRQAVIVTRKLTPEAMPDLNSSTDSAASASAASDVSVESTEATVCTVTLEKMSAGLGFSLEGGKGSLHGDKPLTINRIFKGAASEQSETVQPGDEILQLGGTAMQGLTRFEAWNIIKALPDGPVTIVIRRKSLQSKETTAAGDS

SEQ ID NO: 33 - protein, 1134 aa, Homo sapiens
MENLPFPLKLLSASSLNTPSSTPWVLDIFLTLVFALGFFFLLLPYFSYLRCDNPPSPSPKKRKRHLVSQRPAGRRGRPRGRMKNHSLRACRECPRGLEETWDLLSQLQSLLGPHLEKGDFGQLSGPDPPGEVGKRTPDGASRSSHEPTEDAAPIVSPLASPDPRTKHPQDLASTPPPGPMTTSVSSLSASQPPEPSLLLEHPSPEPPALFPHPPRTPDPLACSPPPPKGFTPPPLRDSTLLTPSHCDSVALPLDTVPQSLSPREDLAASVPGISGLGGSNSQVSALSWSQETTKTWCVFNSSVQQDHLSRQRDTTMSPLLFQAQPLSHLEPESQPFISSTPQFWPTPMAQAEAQAHLQSSFPVLSPAFLSPMKNTGVACPASQNKVQALSLPETQHPERPLLKKQLEGGLALPSRVQKSQDVFSVSTPNLPQERLTSILPENFPVSPELWRQLEQHMGQRGRIQESLDLMQLQDELPGTSQAKGKPRPWQSSTSTGESSKEAQTVKFQLERDPCPHLGQILGETPQNLSRGMESFPGKVLGATSEESERNLRKPLRSDSGSDLLRRTERNHIENILKAHMSRKLGQTNEGLIPVSVRRSWLAVNQAFPVSNTHVKTSNLAAPKSRKACVNTAQVLSFLEPCTQQVLGAHIVRFWAKHRWGLPLRVLKPIQCFQLEKVSSLSLIQLAGPSSDTCESGAGSKVEVATFLGEPPMASLRKQVLTKPSVHMPERLQASSPACKQFQRAPRGIPSSNDHGSLKAPTAGQEGRWPSKPLTYSLTGSTQQSRSLGAQSSRAGETREAVPQPTVPLGTCMRANLQATSEDVRGFKAPGASKSSLLPRMSVSQDPRKLCLMEEAVSEFEPGKATKSETQPQVSATVVLLPDGQASVVPHASENLASQVPQGHLQSMPTGNMQASQELCDLMSARRSNMGHKEPRNPNCQGSCKSQSPMFPPTHKRENSRKPNLEKHEEMFQGLRTPQLTPGRKTEDTRQNEGVQLLPSKKQPPSISHFGENIKQFFQTIFSKKERKPAPVTAESQKTVKNRSCVYGSSAEAERLMTAVGQILEENMSLCHARHASKVNQQRQQFQAPVCGFPCNHRHPFYSEHSRMLSYAASSQQATLKNQSRPNRDRQIRDQ

SEQ ID NO: 34 - protein, 962 aa, Homo sapiens
MEDCNVHSAASILASVKEQEARFERLTRALEQERRHVALQLERAQQPGMVSGGMGSGQPLPMAWQQLVLQEQSPGSQASLATMPEAPDVLEETVTVEEDPGTPTSHVSIVTSEDGTTRRTETKVTKTVKTVTTRTVRQVPVGPDGLPLLDGGPPLGPFADGALDRHFLLRGGGPVATLSRAYLSSGGGFPEGPEPRDSPSYGSLSRGLGMRPPRAGPLGPGPGDGCFTLPGHREAFPVGPEPGPPGGRSLPERFQAEPYGLEDDTRSLAADDEGGPELEPDYGTATRRRPECGRGLHTRAYEDTADDGGELADERPAFPMVTAPLAQPERGSMGSLDRLVRRSPSVDSARKEPRWRDPELPEVLAMLRHPVDPVKANAAAYLQHLCFENEGVKRRVRQLRGLPLLVALLDHPRAEVRRRACGALRNLSYGRDTDNKAAIRDCGGVPALVRLLRAARDNEVRELVTGTLWNLSSYEPLKMVIIDHGLQTLTHEVIVPHSGWEREPNEDSKPRDAEWTTVFKNTSGCLRNVSSDGAEARRRLRECEGLVDALLHALQSAVGRKDTDNKSVENCVCIMRNLSYHVHKEVPGADRYQEAEPGPLGSAVGSQRRRRDDASCFGGKKAKEEWFHQGKKDGEMDRNFDTLDLPKRTEAAKGFELLYQPEVVRLYLSLLTESRNFNTLEAAAGALQNLSAGNWMWATYIRATVRKERGLPVLVELLQSETDKVVRAVAIALRNLSLDRRNKDLIGSYAMAELVRNVRNAQAPPRPGACLEEDTVVAVLNTIHEIVSDSLDNARSLLQARGVPALVALVASSQSVREAKAASHVLQTVWSYKELRGTLQKDGWTKARFQSAAATAKGPKGALSPGGFDDSTLPLVDKSLEGEKTGSRDVIPMDALGPDGYSTVDRRERRPRGASSAGEASEKEPLKLDPSRKAPPPGPSRPAVRLVDAVGDAKPQPVDSWV

SEQ ID NO: 35 - protein, 2261 aa, Homo sapiens
MACWPQLRLLLWKNLTFRRRQTCQLLLEVAWPLFIFLILISVRLSYPPYEQHECHFPNKAMPSAGTLPWVQGIICNANNPCFRYPTPGEAPGVVGNFNKSIVARLFSDARRLLLYSQKDTSMKDMRKVLRTLQQIKKSSSNLKLQDFLVDNETFSGFLYHNLSLPKSTVDKMLRADVILHKVFLQGYQLHLTSLCNGSKSEEMIQLGDQEVSELCGLPREKLAAAERVLRSNMDILKPILRTLNSTSPFPSKELAEATKTLLHSLGTLAQELFSMRSWSDMRQEVMFLTNVNSSSSSTQIYQAVSRIVCGHPEGGGLKIKSLNWYEDNNYKALFGGNGTEEDAETFYDNSTTPYCNDLMKNLESSPLSRIIWKALKPLLVGKILYTPDTPATRQVMAEVNKTFQELAVFHDLEGMWEELSPKIWTFMENSQEMDLVRMLLDSRDNDHFWEQQLDGLDWTAQDIVAFLAKHPEDVQSSNGSVYTWREAFNETNQAIRTISRFMECVNLNKLEPIATEVWLINKSMELLDERKFWAGIVFTGITPGSIELPHHVKYKIRMDIDNVERTNKIKDGYWDPGPRADPFEDMRYVWGGFAYLQDVVEQAIIRVLTGTEKKTGVYMQQMPYPCYVDDIFLRVMSRSMPLFMTLAWIYSVAVIIKGIVYEKEARLKETMRIMGLDNSILWFSWFISSLIPLLVSAGLLVVILKLGNLLPYSDPSVVFVFLSVFAVVTILQCFLISTLFSRANLAAACGGIIYFTLYLPYVLCVAWQDYVGFTLKIFASLLSPVAFGFGCEYFALFEEQGIGVQWDNLFESPVEEDGFNLTTSVSMMLFDTFLYGVMTWYIEAVFPGQYGIPRPWYFPCTKSYWFGEESDEKSHPGSNQKRISEICMEEEPTHLKLGVSIQNLVKVYRDGMKVAVDGLALNFYEGQITSFLGHNGAGKTTTMSILTGLFPPTSGTAYILGKDIRSEMSTIRQNLGVCPQHNVLFDMLTVEEHIWFYARLKGLSEKHVKAEMEQMALDVGLPSSKLKSKTSQLSGGMQRKLSVALAFVGGSKVVILDEPTAGVDPYSRRGIWELLLKYRQGRTIILSTHHMDEADVLGDRIAIISHGKLCCVGSSLFLKNQLGTGYYLTLVKKDVESSLSSCRNSSSTVSYLKKEDSVSQSSSDAGLGSDHESDTLTIDVSAISNLIRKHVSEARLVEDIGHELTYVLPYEAAKEGAFVELFHEIDDRLSDLGISSYGISETTLEEIFLKVAEESGVDAETSDGTLPARRNRRAFGDKQSCLRPFTEDDAADPNDSDIDPESRETDLLSGMDGKGSYQVKGWKLTQQQFVALLWKRLLIARRSRKGFFAQIVLPAVFVCIALVFSLIVPPFGKYPSLELQPWMYNEQYTFVSNDAPEDTGTLELLNALTKDPGFGTRCMEGNPIPDTPCQAGEEEWTTAPVPQTIMDLFQNGNWTMQNPSPACQCSSDKIKKMLPVCPPGAGGLPPPQRKQNTADILQDLTGRNISDYLVKTYVQIIAKSLKNKIWVNEFRYGGFSLGVSNTQALPPSQEVNDAIKQMKKHLKLAKDSSADRFLNSLGRFMTGLDTKNNVKVWFNNKGWHAISSFLNVINNAILRANLQKGENPSHYGITAFNHPLNLTKQQLSEVALMTTSVDVLVSICVIFAMSFVPASFVVFLIQERVSKAKHLQFISGVKPVIYWLSNFVWDMCNYVVPATLVIIIFICFQQKSYVSSTNLPVLALLLLLYGWSITPLMYPASFVFKIPSTAYVVLTSVNLFIGINGSVATFVLELFTDNKLNNINDILKSVFLIFPHFCLGRGLIDMVKNQAMADALERFGENRFVSPLSWDLVGRNLFAMAVEGVVFFLITVLIQYRFFIRPRPVNAKLSPLNDEDEDVRRERQRILDGGGQNDILEIKELTKIYRRKRKPAVDRICVGIPPGECFGLLGVNGAGKSSTFKMLTGDTTVTRGDAFLNKNSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVLKEKHRNMLQYQLPSSLSSLARIFSILSQSKKRLHIEDYSVSQTTLDQVFVNFAKDQSDDDHLKDLSLHKNQTVVDVAVLTSFLQDEKVKESYV

SEQ ID NO: 36 - protein, 403 aa, Homo sapiens [sequence omitted]